(19) United States
(12) Patent Application Publication
(10) Pub. No.: US 2017/0058278 A1
(43) Pub. Date: Mar. 2, 2017

(54) COMPOSITIONS AND METHODS OF USING SAME FOR CONTROLLING PATHOGENICALLY INFECTED MOSQUITOES

(71) Applicant: Forrest Innovations Ltd., Caesarea (IL)

(72) Inventors: Nitzan PALDI, Moshav Bar Giora (IL); Humberto Freire BONCRISTIANI JUNIOR, Odenton, MD (US); Eyal MAORI, Rishon-LeZion (IL); Avital WEISS, Karkur (IL); Emerson Soares BERNARDES, Sao Paulo (BR)

(21) Appl. No.: 15/307,858

(22) PCT Filed: May 4, 2015
(86) PCT No.: PCT/IL2015/050464

§ 371 Date: Oct. 31, 2016

Related U.S. Application Data
(60) Provisional application No. 61/988,234, filed on May 4, 2014.
(60) Provisional application No. 61/988,235, filed on May 4, 2014.
(60) Provisional application No. 61/988,237, filed on May 4, 2014.
(60) Provisional application No. 61/988,236, filed on May 4, 2014.
(60) Provisional application No. 61/988,246, filed on May 4, 2014.

(51) Int. Cl. C12N 15/113; A01N 25/10; A01N 25/02; A01N 57/16; A01N 25/00

(52) U.S. Cl. CPC C12N 15/113; A01N 57/16; C12N 15/1138; A01N 25/006; A01N 25/02; A01N 25/10; C12N 2310/14; C12N 2320/35; C12N 2320/30

(57) ABSTRACT

A method of controlling a pathogenically infected mosquito is disclosed. The method comprising administering to a larva of a mosquito an isolated nucleic acid agent comprising a nucleic acid sequence which specifically downregulates an expression of at least one mosquito pathogen resistance gene product of the mosquito, wherein downregulation of the expression of the at least one mosquito pathogen resistance gene in the larvae renders an adult stage of the mosquito lethally susceptible to the pathogen, thereby controlling the pathogenically infected mosquito.

### FIELD AND BACKGROUND OF THE INVENTION

The present invention, in some embodiments thereof, relates to isolated nucleic acid agents, and, more particularly, but not exclusively, to the use of same for controlling pathogenically infected mosquitoes.

Mosquitoes Harbor, Replicate and Transmit Human Pathogenic Viruses

Insects are among the most diverse and numerous animals on earth and populate almost every habitat. As agricultural pests, they cause severe economic losses by damaging and killing crops, but insects also pose an important threat to human and animal health. Insects are vectors for numerous pathogens, including viruses, bacteria, protozoa and nematodes.

Over 500 arthropod-borne viruses (arboviruses) have been identified, among which approximately 100 are harmful to humans. Arboviruses cause some of the most serious and feared human infectious diseases, such as hemorrhagic fevers and encephalitides, yet their infections of arthropod vectors, which are essential links in their transmission cycles, are almost always nonpathogenic and persistent for the life of the mosquito or tick. However, there is evidence that some parasites manipulate the behavior of their vectors to enhance pathogen transmission. For example, the malaria mosquito Anopheles gambiae, infected with transmissible sporozoite stages of the human malaria parasite Plasmodium falciparum, takes larger and more frequent blood meals than uninfected mosquitoes or those infected with non-transmissible oocyst forms. This parasite-mediated manipulation of behavior in An. gambiae is likely to facilitate parasite transmission.

The suite of factors that allow an arthropod that has encountered a pathogen to become infected and to transmit a particular pathogen once it encounters a susceptible host is defined as the arthropod's vector competence for that pathogen.

The process of vector infection begins when the pathogen enters the mosquito within a blood meal containing sufficient numbers of the pathogen to ensure some will encounter the epithelium where the blood has been deposited in the arthropod's midgut. The pathogen must be able to cross the epithelium that has been termed the midgut infection barrier (MIB). Once in the epithelium the pathogen must replicate, cross the epithelium and escape the midgut into the hemocoel in a process termed the midgut escape barrier (MEB). The pathogen then must replicate in various mosquito tissues but ultimately some sufficient quantity of the pathogen must invade the mosquito's salivary glands in a process overcoming the salivary gland infection barrier (SIB). There the pathogen replicates and ultimately must escape the salivary gland in the process described as the salivary gland escape barrier (SEB) upon subsequent blood feeding when it is injected into a susceptible animal host to complete the transmission cycle. This entire process can take several days to complete in the mosquito during a period called the extrinsic incubation period (EIP). Along the way there are other arthropod related factors including various barriers to the pathogen that may also influence the pathogen and the arthropod's vector competence. The pathogen encounters arthropod digestive enzymes and digestive processes, intracellular processes and the arthropod's immune system.

Some Mosquitoes are Naturally Able to Restrict Virus Replication by Mounting a Strong Host Defense Response to Viral Infection

Horizontal arbovirus infection of the vector is established upon blood-feeding of a susceptible female mosquito on a viremic vertebrate host. Within the insect vector, arboviruses have a complex life cycle that includes replication in the midgut, followed by systemic dissemination via the hemolymph and replication in the salivary glands. Transmission of an arbovirus to a naive vertebrate host during blood-feeding requires high viral titers in the saliva. Anatomical and immunological barriers affect the ability of the virus to reach such titers and thus to accomplish successful transmission to a naive host.

Despite efficient replication, arboviruses do not cause pathology suggesting that the insect immune system restricts virus infection to non-pathogenic levels. Innate immunity provides the first line of defense against microbial invaders and is defined by its rapid activation following pathogen recognition by germline-encoded receptors. These receptors recognize small molecular motifs that are conserved among classes of microbes, but are absent from the host, such as bacterial cell wall components and viral double-stranded (ds) RNA. Collectively, these motifs are called pathogen-associated molecular patterns (PAMP).

When exposed to arboviruses, mosquitoes respond with anti-microbial immune pathways like Janus kinase-signal transducer and activator of transcription (JAK/STAT) and Toll pathways, immune deficiency (IMD) and RNA interference (RNAi) machinery.

RNAi is one of the molecular mechanisms for regulation of gene expression generally known as RNA silencing. It has a central role in insect antiviral immunity. It appears to require minimal transcriptional induction, although its activation might induce upregulation of other antiviral genes. Notably, the RNAi response inhibits virus replication without causing death of the infected cell.

Thus, for example, RNAi can eliminate Dengue virus (DENV2) from transgenic mosquitoes expressing an inverted-repeat RNA to trigger the RNAi pathway against the virus. However, arboviruses are able to persistently infect vectors despite being targeted by the RNAi machinery as shown by the presence of 21 nt virus-derived small interfering RNAs (viRNAs) in arbovirus-infected, transmission-competent mosquito vectors [Scott et al. (2010) PLOS Negl Trop Dis 4: e848; Hess et al. (2011) BMC Microbiol 11: 45].

A number of insect pathogenic viruses express a virus-encoded protein suppressor of RNAi (VSR) during replication. Expression of VSRs in insect virus-infected cells results in enhanced virus production, but in most cases these are virulence factors that greatly increase the pathogenicity of the viral infection. For example, temporally induced silencing of the RNAi machinery in Ae. aegypti led to significantly increased SINV (sindbis virus) and DENV2 (Dengue virus) titres combined with increased midgut infection and dissemination rates and a shortened extrinsic incubation period [Campbell et al. (2008) BMC Microbiol 8: 47; Sanchez-Vargas et al. (2009) PLOS Pathog 5: e1000299; Khoo et al. (2010) BMC Microbiol 10: 130].

One of the most widely studied and potent insect virus VSRs is the B2 protein encoded by Flock House virus (FHV; Nodaviridae). The B2 protein is a homodimer and indiscriminately binds to double-stranded RNA (dsRNA) molecules independent of their nucleotide sequences and sizes such as siRNAs duplexes and long dsRNAs, thereby protecting dsRNA from being accessed and processed by dicer2 of the RNAi machinery. A recombinant SINV strain was engineered to express the FHV B2 protein. When mosquitoes were orally infected with SINV-B2, virus titres, midgut infection and escape rates were significantly increased compared to the control virus. Strikingly, SINV-B2 caused high mortality amongst the mosquitoes at 4-6 days post-infection suggesting that RNAi has the potential to protect the vector from pathogenic effects of replicating arboviruses [Myles et al. (2008) Proc Natl Acad Sci USA 105: 19938-19943; Cirimotich et al. (2009) BMC Microbiol 9: 49].

Furthermore, constitutive over-expression of B2 in PUbB2 P61 mosquitoes suppressed the RNAi pathway in the mosquitoes. Two unrelated arboviruses, SINV-TR339eGFP and DENV2-QR94, responded similarly to RNAi suppression in midgut tissue of PUbB2 P61 mosquitoes by producing significantly increased mean virus titres at 7 days pbm [Khoo et al. (2013) Insect Mol Biol. 22(1): 104-14].

Externally Delivered dsRNA can be Effective in Gene Regulation and Provide Phenotypic Effects in Adult and Larvae in Mosquitoes

In studies involving insects, administration (e.g. by direct injections) of in vitro-synthesized dsRNA into virtually any developmental stage can produce loss-of-function mutants [Bettencourt et al. (2002) Insect Molecular Biology 11:267-271; Amdam et al. (2003) BMC Biotechnology 3: 1; Tomoyasu and Denell (2004) Development Genes and Evolution 214: 575-578; Singh et al. (2013) J Insect Sci. 13: 69].

Studies on feeding dsRNA revealed effective gene knockdown effects in many insects, including insects of the orders Hemiptera, Coleoptera, and Lepidoptera. Feeding dsRNA to E. postvittana larvae has been shown to inhibit the expression of the carboxylesterase gene EposCXE1 in the larval midgut and also inhibit the expression of the pheromone-binding protein EposPBP1 in adult antennae [Turner et al. (2006) Insect Molecular Biology 15: 383-391]. The feeding of dsRNA also inhibited the expression of the nitrophorin 2 (NP2) gene in the salivary gland of R. prolixus, leading to a shortened coagulation time of plasma [Araujo et al. (2006) Insect Biochemistry and Molecular Biology 36: 683-693].

Direct spray of dsRNA on newly hatched Ostrinia furnalalis larvae has been reported by Wang et al. [Wang et al. (2011) PloS One 6: e18644]. The studies have shown that after spraying dsRNAs (50 ng/μL) of the DS10 and DS28 genes (i.e. chymotrypsin-like serine protease C3 (DS10) and an unknown protein (DS28), respectively) on the newly hatched larvae placed on the filter paper, the larval mortalities were around 40-50%, whereas, after dsRNAs of ten genes were sprayed on the larvae along with artificial diet, the mortalities were significantly higher to the extent of 73-100%. It was proposed through these results that in a lepidopteron insect, dsRNAs are able to penetrate the integument and could retread larval developmental ultimately leading to death [Katoch (2013) Appl Biochem Biotechnol., 171(4): 847-73].

In mosquitoes, RNAi method using chitosan/dsRNA self-assembled nanoparticles to mediate gene silencing through larval feeding in the African malaria mosquito (Anopheles gambiae) was shown [Zhang et al. (2010) Insect Molecular Biology (2010) 19(5): 683-693]. Oral-delivery of dsRNAs to larvae of the yellow fever mosquito, Ae. aegypti was also shown to be insecticidal. It was found that a relatively brief soaking in dsRNA, without the use of transfection reagents or dsRNA carriers, was sufficient to induce RNAi, and can either stunt growth or kill mosquito larvae [Singh et al. (2013), supra]. Furthermore, dsRNA targeting RNAi pathway genes were described to increase Dengue virus (DENV) replication in the Ae. Aegypti mosquito and to decrease the extrinsic incubation period required for virus transmission [Sanchez-Vargas et al. (2009), supra]. The authors describe targeting the sequence of the gene AAEL011753 (r2d2) by 76-575, which is one of the proteins of the silencing complex.

One method of introducing dsRNA to the larvae is by dehydration. Specifically, larvae are dehydrated in a NaCl solution and then rehydrated in water containing double-stranded RNA. This process is suggested to induce gene silencing in mosquito larvae.

A recently published RNA seq analysis describing mosquito transcriptional profiles during DENV infection show that all transcripts representing immunity-related genes with differential accumulation in midgut samples were always more abundant in control than DENV mosquitoes, supporting the conclusion that there is a suppression of the insect immune system following infection. This result may reflect the general ‘DENV downregulation trend” observed. A similar pattern was seen in carcass samples at early time points postinfection, but the opposite was observed at 14 days post infection (dpi), reflecting a possible change in immune modulation during the course of the infection [Bonizzoni et al. (2012) PLoS ONE 7(11): e50512].

U.S. Patent Application Nos. 20030154508 and 20030140371 provide pesticidal compositions that contain one or more compounds that interact with organic solute transporter/ligand-gated ion channel multifunction polypeptides (e.g. CAATCH protein) in the pest (e.g. mosquito), and/or alter amino acid metabolic pathways, and/or alter ionic homeostasis in the pest (e.g. mosquito). Upon exposure to a target pest, these compositions either compromise pest growth and/or cause the death of the pest. The compositions of U.S. 20030154508 and 20030140371 may contain one or more amino acids and/or amino acid analogs, or alternatively may comprise antibodies, antisense polynucleotides or RNAi.

U.S. Patent Application No. 20090285784 provides dsRNA as insect control agents. Specifically, U.S. 20090285784 provides methods for controlling insect infestation via RNAi-mediated gene silencing, whereby the intact insect cell(s) are contacted with a double-stranded RNA from outside the insect cell(s) and whereby the double-stranded RNA is taken up by the intact insect cell(s). U.S. Patent Application No. 20090010888 provides the use of cytochrome P450 reductase (CPR) as an insecticidal target. Specifically, U.S. 20090010888 provides methods of pest treatment (e.g. mosquitoes) comprising administering an agent (e.g. dsRNA) which is effective in reducing an activity and/or expression of the pest's CPR.

U.S. Patent Application No. 20130011372 provides inactivated microorganisms containing dsRNA molecules capable of inhibiting the functionality of a GPCR receptor and their use as pesticides.

U.S. Patent Application No. 20130137747 provides dsRNA RNA based nanoparticles for insect gene silencing. According to the teachings of U.S. 20130137747 the nanoparticles are orally ingested by the target insect (e.g. mosquito) and trigger gene silencing of a target gene such as insect chitin synthase gene (CHS1 and/or CHS2).

U.S. Patent Application No. 20100011654 provides RNAi for the control of insects and arachnids. U.S. 20100011654 provides dsRNA formulations matching a part of an essential insect gene (e.g. structural proteins, metabolic enzymes, enzymes involved in ion/pH homeostasis and enzymes involved in the transcriptional/translational machinery), causing downregulation of the insect target via RNA interference (RNAi), and consequently causing death, growth arrest or sterility of the insect and/or arachnid.

### SUMMARY OF THE INVENTION

According to an aspect of some embodiments of the present invention there is provided a method of controlling a pathogenically infected mosquito, the method comprising administering to a larva of a mosquito an isolated nucleic acid agent comprising a nucleic acid sequence which specifically downregulates an expression of at least one mosquito pathogen resistance gene product of the mosquito, wherein downregulation of the expression of the at least one mosquito pathogen resistance gene in the larvae renders an adult stage of the mosquito lethally susceptible to the pathogen, thereby controlling the pathogenically infected mosquito.

According to some embodiments of the invention, the mosquito comprises a female mosquito being capable of transmitting a disease to a mammalian organism.

According to some embodiments of the invention, the mosquito is of a species selected from the group consisting of Aedes aegypti, Aedes albopictus and Anopheles gambiae.

According to some embodiments of the invention, the administering comprises feeding, spraying or soaking.

According to some embodiments of the invention, the administering comprises soaking the larva with the isolated nucleic acid agent for about 12-48 hours.

According to some embodiments of the invention, the larva comprises third instar larva.

According to some embodiments of the invention, the method further comprises feeding the larva with the isolated nucleic acid agent until the larva reaches pupa stage.

According to some embodiments of the invention, the pathogenically infected mosquito carries an infection selected from the group consisting of a viral infection, a nematode infection, a protozoa infection and a bacterial infection.

According to some embodiments of the invention, the viral infection is caused by an arbovirus.

According to some embodiments of the invention, the arbovirus is selected from the group consisting of an alphavirus, a flavivirus, a bunyavirus and an orbivirus.

According to some embodiments of the invention, the arbovirus is selected from the group consisting of a La Crosse encephalitis virus, an Eastern equine encephalitis virus, a Japanese encephalitis virus, a Western equine encephalitis virus, a St. Louis encephalitis virus, a Tick-borne encephalitis virus, a Ross River virus, a Venezuelan equine encephalitis virus, a Chikungunya virus, a West Nile virus, a Dengue virus, a Yellow fever virus, a Bluetongue disease virus, a Sindbis Virus and a Rift Valley Fever virus a Colorado tick fever virus, a Murray Valley encephalitis virus, an Oropouche virus and a Flock House virus.

According to some embodiments of the invention, the protozoa infection is caused by a Plasmodium.

According to some embodiments of the invention, the protozoa infection causes malaria.

According to some embodiments of the invention, the nematode infection is caused by a Heartworm (Dirofilaria immitis) or a Wuchereria bancrofti.

According to some embodiments of the invention, the nematode infection causes Heartworm Disease.

According to an aspect of some embodiments of the present invention there is provided a mosquito larva-ingestible compound comprising an isolated nucleic acid agent comprising a nucleic acid sequence which specifically downregulates an expression of at least one mosquito pathogen resistance gene product in a mosquito and a microorganism or algae on which mosquito larva feed.

According to some embodiments of the invention, the mosquito larva-ingestible compound of some embodiments of the invention is formulated as a solution.

According to some embodiments of the invention, the mosquito larva-ingestible compound of some embodiments of the invention is formulated in a solid or semi-solid formulation.

According to some embodiments of the invention, the semi-solid formulation comprises an agarose.

According to some embodiments of the invention, the microorganism is selected from the group consisting of a bacteria and a water surface microorganism.

According to some embodiments of the invention, the mosquito pathogen resistance gene is selected from the group consisting of a RNA interference related gene, a piRNA pathway related gene, an immunity related gene, a metabolism related gene, a cytoskeleton related gene, a cell membrane related gene, a cell motility related gene, an extracellular structure related gene, a post-translational modification related gene, a protein turnover related gene, a chaperone related gene, a signal transduction related gene, a proteolysis related gene, an oxidoreductase activity related gene, a transcription related gene, a translation related gene, a diverse related gene, a transport related gene, a cell-cycle related gene, an energy production and conversion related gene, a chromatin structure and dynamics related gene, a Toll related gene and a JAK/STAT related gene.

According to some embodiments of the invention, the mosquito pathogen resistance gene is selected from the group consisting of AAEL003673 [histone H4], AAEL003689 [histone H4], AAEL003669 [histone H2], AAEL002610 [serine protease], AAEL005004, AAEL011455 [CTLMA12], AAEL007599, AAEL007585 [cathepsin B], AAEL017536 [holotricin], AAEL003603, AAEL007669, AAEL001702, AAEL017571, AAEL015312 [cathepsin B], AAEL012216 [cathepsin B], AAEL008418 [pyrroline-5-carboxylate reductase]), AAEL013857, AAEL000335 [lamin], AAEL003211, AAEL003950 [helicase], AAEL002422 [cytoplasmic polyadenylation element binding protein], AAEL015328, AAEL000652 [GNBPA2], AAEL009178 [GNBPB4], AAEL007064 [GNBPB6], AAEL003253 [CLIPB13B], AAEL001929 [SPZ5], AAEL011608 [PGRPLD], AAEL007696 [REL1A], AAEL015515 [CECG], AAEL004522 [GAM], AAEL015404 [LYSC], AAEL012471 [DOME], AAEL012553 [HOP], AAEL009692 [STAT], AAEL006949 [SOCS16D], AAEL006936 [SOCS16D], AAEL000255 [SOCS44A], AAEL000393 [SOCS], AAEL015099 [SUMO], AAEL011753 (r2d2), AAEL006794 (dcr2), AAEL017251 (ago2), AAEL007823 (Ago3), AAEL013235 (Spn-E), AAEL007698 (AuB), AAEL000709 (Cactus), AAEL007768 (MyD88), AAEL003832, AAEL007562, AAEL000598 and AAEL010179.

According to some embodiments of the invention, the mosquito pathogen resistance gene is selected from the group consisting of AAEL007768 (MyD88), AAEL000709 (Cactus), AAEL007698 (AuB), AAEL003832, AAEL007562, Rel1A (AAEL007696), AAEL000598 and AAEL010179.

According to an aspect of some embodiments of the present invention there is provided an isolated nucleic acid agent comprising a polynucleotide expressing a nucleic acid sequence which specifically downregulates an expression of at least one mosquito pathogen resistance gene selected from the group consisting of AAEL003673 [histone H4], AAEL003689 [histone H4], AAEL003669 [histone H2], AAEL002610 [serine protease], AAEL005004, AAEL011455 [CTLMA12], AAEL007599, AAEL007585 [cathepsin B], AAEL017536 [holotricin], AAEL003603, AAEL007669, AAEL001702, AAEL017571, AAEL015312 [cathepsin B], AAEL012216 [cathepsin B], AAEL008418 [pyrroline-5-carboxylate reductase]), AAEL013857, AAEL000335 [lamin], AAEL003211, AAEL003950 [helicase], AAEL002422 [cytoplasmic polyadenylation element binding protein], AAEL015328, AAEL000652 [GNBPA2], AAEL009178 [GNBPB4], AAEL007064 [GNBPB6], AAEL003253 [CLIPB13B], AAEL001929 [SPZ5], AAEL011608 [PGRPLD], AAEL007696 [REL1A], AAEL015515 [CECG], AAEL004522 [GAM], AAEL015404 [LYSC], AAEL012471 [DOME], AAEL012553 [HOP], AAEL009692 [STAT], AAEL006949 [SOCS16D], AAEL006936 [SOCS16D], AAEL000255 [SOCS44A], AAEL000393 [SOCS], AAEL015099 [SUMO], AAEL011753 (r2d2), AAEL006794 (dcr2), AAEL017251 (ago2), AAEL007823 (Ago3), AAEL013235 (Spn-E), AAEL007698 (AuB), AAEL000709 (Cactus), AAEL007768 (MyD88), AAEL003832, AAEL007562, AAEL010179 and AAEL000598.

According to an aspect of some embodiments of the present invention there is provided an isolated nucleic acid agent comprising a polynucleotide expressing a nucleic acid sequence which specifically downregulates an expression of at least one mosquito pathogen resistance gene selected from the group consisting of AAEL007768 (MyD88), AAEL000709 (Cactus), AAEL007698 (AuB), AAEL003832, AAEL007562, Rel1A (AAEL007696), AAEL010179 and AAEL000598.

According to an aspect of some embodiments of the present invention there is provided a nucleic acid construct comprising a nucleic acid sequence encoding the isolated nucleic acid agent of some embodiments of the invention.

According to an aspect of some embodiments of the present invention there is provided a cell comprising the isolated nucleic acid agent or the nucleic acid construct of some embodiments of the invention.

According to some embodiments of the invention, the cell is selected from the group consisting of a bacterial cell and a cell of a water surface microorganism.

According to an aspect of some embodiments of the present invention there is provided a mosquito larva-ingestible compound comprising the cell of some embodiments of the invention.

According to some embodiments of the invention, the nucleic acid agent is a dsRNA.

According to some embodiments of the invention, the dsRNA is a naked dsRNA.

According to some embodiments of the invention, the dsRNA comprises a carrier.

According to some embodiments of the invention, the carrier comprises a polyethyleneimine (PEI).

According to some embodiments of the invention, the dsRNA is effected at a dose of 0.001-1 μg/μL for soaking or at a dose of 1 pg to 10 μg/larvae for feeding.

According to some embodiments of the invention, the dsRNA is selected from the group consisting of SEQ ID NOs: 1315-1324 and 1330.

According to some embodiments of the invention, the dsRNA is selected from the group consisting of siRNA, shRNA and miRNA.

According to some embodiments of the invention, the nucleic acid sequence is greater than 15 base pairs in length.

According to some embodiments of the invention, the nucleic acid sequence is 19 to 25 base pairs in length.

According to some embodiments of the invention, the nucleic acid sequence is 30-100 base pairs in length.

According to some embodiments of the invention, the nucleic acid sequence is 100-800 base pairs in length.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of the regulation of putative Toll signaling pathway genes by dengue virus infection. Red color indicates infection responsive up-regulation and green color indicate infection responsive downregulation. Non-colored gene boxes indicate lack of infection responsive regulation.

FIG. 2 is a schematic illustration of the JAK/STAT signaling cascade. The core JAK-STAT pathway factors identified in Drosophila and their putative roles in the cascade are shown.

FIGS. 3A-D are schematic illustrations of mosquito immune signaling and RNAi pathways. FIG. 3A, in Toll pathway signaling, detection of pathogen-derived ligands by pattern recognition receptors (PRRs) triggers signaling through the adaptor protein MyD88, resulting in degradation of Cactus, which in turn leads to activation of transcription of Toll-pathway regulated genes. FIG. 3B, the IMD pathway is activated by ligand binding to PGRP-LCs and -LEs. This binding triggers signaling through IMD and various caspases and kinases, leading to a functional split in the pathway. Both pathway branches lead to an activated form of Rel2 which translocates to the nucleus and activate IMD-regulated transcription. FIG. 3C, the JAK-STAT pathway is triggered by Unpaired (Upd) binding to the receptor Dome, activating the receptor-associated Hop Janus kinases, which results in dimerization of phosphorylated-STAT and its translocation to the nucleus to activate JAK-STAT-regulated transcription. FIG. 3D, the exogenous siRNA pathway is activated when virus-derived long dsRNA is recognized, cleaved by Dcr2 into siRNAs and loaded onto the multi-protein RISC complex, where it is degradated. Sensing of viral dsRNA by Dcr2 also activates TRAF, leading to Rel2 cleavage and activation via a distinct pathway. Rel2 activates transcription of Vago, a secreted peptide which subsequently triggers JAK-STAT pathway signaling. Incorporated from Sim et al., Viruses 2014, 6, 4479-4504.

FIG. 4 is a flowchart illustration depicting introduction of dsRNA into mosquito larvae. In short, third instar larvae were treated (in groups of 100 larvae) in a final volume of 3 mL of dsRNA solution in autoclaved water (0.1 to 0.2 μg/μL, depending on the target. See Table 4B). The control group was kept in 3 ml sterile water only. After soaking in the dsRNA solutions for 24 hr at 27° C., the larvae were transferred into a new container (300 larvae/1500 mL of chlorine-free tap water), and were provided with both agarose cubes containing 300 μg of dsRNA once a day (for a total of two days) and 6 mg/100 mL lab dog/cat diet (Purina Mills) suspended in water. As pupae developed, they were transferred to individual vials to await eclosion and sex sorting, followed by feeding with virus infected blood.

FIGS. 5A-B are graphs illustrating the comparison of two methods of in vivo infection with Flock house virus. FIG. 5A, supernatants from FHV-infected S2-Drosophila cells were diluted with defibrinated sheep blood and exposed to adult females of Aedes aegypti through a pork gut membrane on a water-jacketed membrane feeder for 20 minutes. Control mosquitoes were fed uninfected blood. At the indicated timepoints postinfection, 5-7 individual mosquitoes were collected and analyzed for FHV viral load by qPCR. FIG. 5B, supernatants from FHV-infected S2-Drosophila cells were diluted (v/v) in a 10%-solution of sugar, and the mixture were adsorved in filter paper. The filters were exposed to Ae. aegypti females for 20 minutes. Control mosquitoes were exposed to sugar only. The viral loads were determined as described in FIG. 5A. Of note, FIGS. 5A and 5B show the typical profile of FHV infection in mosquitoes.

FIGS. 6A-B are graphs illustrating the relative expression of MyD88 and Rel1A gene in Ae. aegypti mosquitoes infected with Flock house virus. Females Ae. aegypti mosquitoes were infected with a mixture of defibrinated sheep blood and supernatants from FHV-infected S2-Drosophila for 20 minutes. Control mosquitoes were fed with uninfected blood. At the indicated timepoints postinfection, 5-7 individual mosquitoes were collected and analyzed for the mRNA levels of MyD88 and Rel1A by qPCR. Data represents the mean plus standard deviation of the 5-7 mosquitoes analyzed individually. *p<0.05; ***p<0.001; ****p<0.00001; in Sidak's multiple comparisons test.

FIGS. 7A-E are graphs illustrating that feeding larvae with different dsRNAs increases the viral load of Flock house virus by adult Ae. aegypti mosquitoes. Larvae from A. aegypti RJ strain (3^{th} instar) were soaked for 24 hours with the indicated dsRNA (0.1 to 0.2 μg/μL) or only in water. After soaking in the dsRNA solutions for 24 hr at 27° C., the larvae were transferred into a new container (300 larvae/1500 mL of chlorine-free tap water), and were provided with agarose cubes containing 300 μg of dsRNA once a day (for a total of two days) and then reared until adult stage. Unfed three to five-day-old females were exposed to a mixture of defibrinated sheep blood and supernatants from FHV-infected S2-Drosophila for 20 minutes. At two hours after the exposure of mosquitoes to FHV, individual mosquitoes were collected and analyzed for viral loads by qPCR method. The dots and squares represent individual mosquitoes and the mean among them. *p<0.01; **p<0.001 (Student t test).

FIG. 8 is a table showing the mortality rate of dsRNA-treated mosquitoes and water control group at 15 days postinfection. Of note, the high mortality rate of Rel1A and cactus-treated mosquitoes.

FIGS. 9A-C are graphs illustrating that feeding larvae with MyD88 dsRNA increases viral load of Flock house virus and susceptibility by adult A. aegypti mosquitoes. Larvae from A. aegypti RJ strain (3^{th} instar) were soaked for 24 hours in 0.2 μg/mL of MYD88 dsRNA or only in water. After soaking in the dsRNA solutions for 24 hr at 27° C., the larvae were transferred into a new container (300 larvae/1500 mL of chlorine-free tap water), and were provided with agarose cubes containing 300 μg of dsRNA once a day (for a total of two days) and then larvae were reared until adult stage. Unfed three to five-day-old females were exposed to a mixture of defibrinated sheep blood and supernatants from FHV-infected S2-Drosophila for 20 minutes. At two hours after the exposure of mosquitoes to FHV, individual mosquitoes were collected and analyzed for viral loads by qPCR method using primers specifically designed for FHV RNA-1, and normalized by the mosquito endogenous control tubulin. (FIG. 9A) Number of infected mosquitoes after 2 hours, 7 days and 15 days postinfection with Flock house virus (treatment with dsRNA MyD88). (FIG. 9B) Individual mosquitoes were collected and analyzed for viral loads by qPCR, using primers specifically designed for FHV RNA-1 (FIG. 9C) or for MyD88 mRNA expression by qPCR. **p<0.001 (Two-way Anova followed by Sidak's test).

FIGS. 10A-C are graphs illustrating that feeding REL1A dsRNA to larvae affects the susceptibility of adult A. aegypti mosquitoes to Flock house virus infection. Larvae from A. aegypti RJ strain (3^{th} instar) were soaked for 24 hours in 0.1 μg/mL REL1A dsRNA or only in water. After soaking in the dsRNA solutions for 24 hours at 27° C., the larvae were transferred into a new container (300 larvae/1500 mL of chlorine-free tap water), and were provided with agarose cubes containing 300 μg of dsRNA once a day (for a total of two days) and then larvae were reared until adult stage. Unfed three to five-day-old females were exposed to a mixture of defibrinated sheep blood and supernatants from FHV-infected S2-Drosophila for 20 minutes. At two hours, 7 days and 15 days after the exposure of mosquitoes to FHV, individual mosquitoes (live and dead) were collected and analyzed for viral loads and mRNA expression level by qPCR, using primers specifically designed for FHV RNA-1, and normalized by the mosquito endogenous control tubulin. (FIG. 10A) Number of infected mosquitoes after 2 hours, 7 days and 15 days postinfection with Flock house virus (treatment with dsRNA REL1A). (FIG. 10B) Individual mosquitoes were collected and analyzed for viral loads by qPCR, using primers specifically designed for FHV RNA-1 (FIG. 10C) or for REL1A mRNA expression by qPCR. ****p<0.0001 (Two-way Anova followed by Sidak's test).

### DESCRIPTION OF SPECIFIC EMBODIMENTS OF THE INVENTION

The present invention, in some embodiments thereof, relates to isolated nucleic acid agents, and, more particularly, but not exclusively, to the use of same for controlling pathogenically infected mosquitoes.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 1315 is expressed in a DNA sequence format (e.g., reciting T for thymine), but it can refer to either a DNA sequence that corresponds to an endo 1,4 beta glucanase nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

Mosquitoes pose an important threat to human and animal health. Mosquitoes are vectors for numerous pathogens, including viruses, bacteria, protozoa and nematodes. In fact over 500 arthropod-borne viruses (arboviruses) have been identified, among which approximately 100 are harmful to humans. While mosquitoes transmit these harmful pathogens, arboviruses do not cause pathology in mosquitoes suggesting that the insect's immune system restricts virus infection to non-pathogenic levels, thus allowing the pathogen to replicate in the mosquito and be transmitted to humans and animals.

While reducing the present invention to practice, the present inventors have uncovered that feeding dsRNA to mosquito larvae, wherein the dsRNA specifically downregulates an expression of at least one mosquito pathogen resistance gene, makes adult mosquitoes of the larvae more susceptible to a pathogen which they carry and exterminates pathogenically infected mosquitoes.

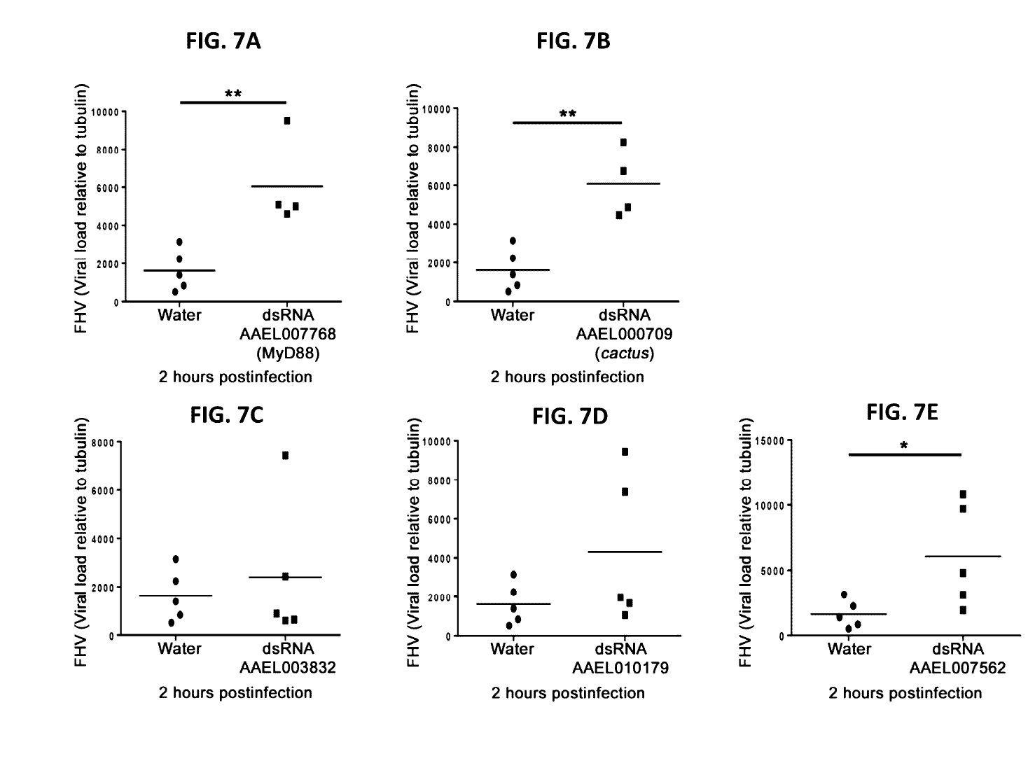

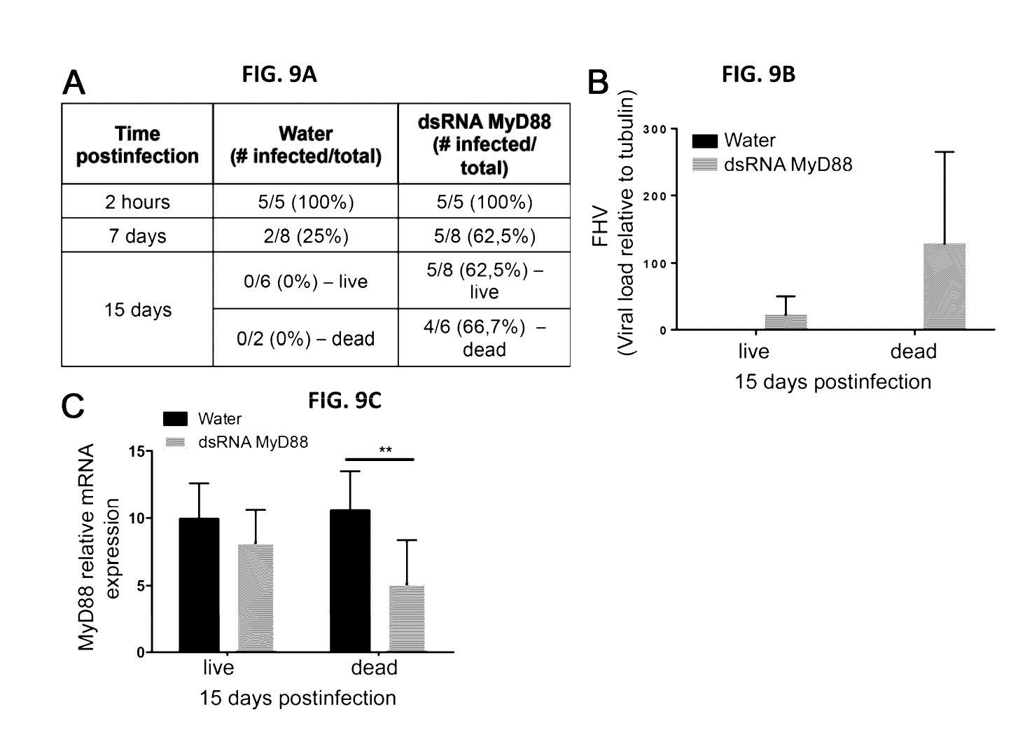

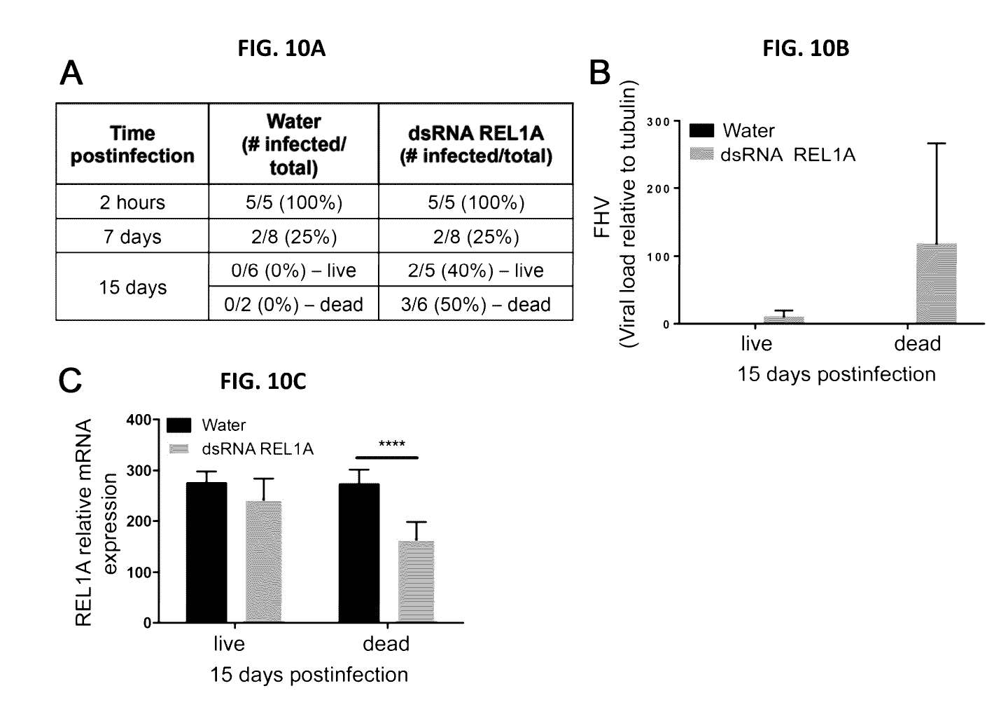

Specifically, the present inventors have shown that soaking mosquito larvae in dsRNA targeting specific genes (e.g. MyD88 (AAEL007768), cactus (AAEL000709), AAEL003832, AAEL010179, AAEL007562) for 24 hours followed by feeding the larvae with agarose cubes containing dsRNA for two more days (until they reach pupa stage) efficiently increased the virus titer at 2 hours postinfection (FIGS. 7A-E). In addition, decreased expression level of the target genes MYD88 (FIG. 9C) and Real1A (FIG. 10C) and higher viral load associated with higher mortality rate in adult mosquitoes 15 days postinfection (FIGS. 9A-B and 10A-B) was illustrated.

The present inventors postulate that downregulating genes which are involved in mosquito pathogen resistance, including genes whose products are involved in RNA interference, piRNA pathway, immunity, metabolism, cytoskeleton, cell membrane, cell motility, extracellular structure, post-translational modification, protein turnover, chaperone, signal transduction, proteolysis, oxidoreductase activity, transcription, translation, diverse, transport, cell-cycle, energy production and conversion, chromatin structure and dynamics, and Toll and JAK/STAT pathways, can be used for extermination of pathogenically infected mosquitoes. Specifically, the present inventors uncovered that downregulating these genes by feeding dsRNA to mosquito larvae makes adult stage of these mosquitoes more susceptible to pathogenic infection that they carry (e.g. viral infection) and only those mosquitoes that contract the pathogen infection are killed.

Thus, according to one aspect of the present invention there is provided a method of controlling a pathogenically infected mosquito, the method comprising administering to a larva of a mosquito an isolated nucleic acid agent comprising a nucleic acid sequence which specifically downregulates an expression of at least one mosquito pathogen resistance gene product of the mosquito, wherein downregulation of the expression of the at least one mosquito pathogen resistance gene in the larva renders an adult stage of the mosquito lethally susceptible to the pathogen, thereby controlling the pathogenically infected mosquito.

As used herein the term “controlling” refers to managing the population of mosquitoes to reduce their damage to human health, economies, and enjoyment. According to some embodiments of the invention, mosquito management is typically effected using an agent for exterminating (e.g. destroying/killing mosquitoes) or reducing a population of mosquitoes.

The term “mosquito” or “mosquitoes” as used herein refers to an insect of the family Culicidae. The mosquito of the invention may include an adult mosquito, a mosquito larva, a pupa or an egg thereof.

An adult mosquito is defined as any of slender, long-legged insect that has long proboscis and scales on most parts of the body. The adult females of many species of mosquitoes are blood-eating pests. In feeding on blood, adult female mosquitoes transmit harmful diseases to humans and other mammals.

A mosquito larvae is defined as any of an aquatic insect which does not comprise legs, comprises a distinct head bearing mouth brushes and antennae, a bulbous thorax that is wider than the head and abdomen, a posterior anal papillae and either a pair of respiratory openings (in the subfamily Anophelinae) or an elongate siphon (in the subfamily Culicinae) borne near the end of the abdomen.

Typically, a mosquito's life cycle includes four separate and distinct stages: egg, larva, pupa, and adult. Thus, a mosquito's life cycle begins when eggs are laid on a water surface (e.g. Culex, Culiseta, and Anopheles species) or on damp soil that is flooded by water (e.g. Aedes species). Most eggs hatch into larvae within 48 hours. The larvae live in the water feeding on microorganisms and organic matter and come to the surface to breathe. They shed their skin four times growing larger after each molting and on the fourth molt the larva changes into a pupa. The pupal stage is a resting, non-feeding stage of about two days. At this time the mosquito turns into an adult. When development is complete, the pupal skin splits and the mosquito emerges as an adult. According to one embodiment, the mosquitoes are of the sub-families

Anophelinae and Culicinae. According to one embodiment, the mosquitoes are of the genus Culex, Culiseta, Anopheles and Aedes. Exemplary mosquitoes include, but are not limited to, Aedes species e.g. Aedes aegypti, Aedes albopictus, Aedes polynesiensis, Aedes australis, Aedes cantator, Aedes cinereus, Aedes rusticus, Aedes vexans; Anopheles species e.g. Anopheles gambiae, Anopheles freeborni, Anopheles arabiensis, Anopheles funestus, Anopheles gambiae Anopheles moucheti, Anopheles balabacensis, Anopheles baimaii, Anopheles culicifacies, Anopheles dirus, Anopheles latens, Anopheles leucosphyrus, Anopheles maculatus, Anopheles minimus, Anopheles fluviatilis s.l., Anopheles sundaicus Anopheles superpictus, Anopheles farauti, Anopheles punctulatus, Anopheles sergentii, Anopheles stephensi, Anopheles sinensis, Anopheles atroparvus, Anopheles pseudopunctipennis, Anopheles bellator and Anopheles cruzii; Culex species e.g. C. annulirostris, C. antennatus, C. jenseni, C. pipiens, C. pusillus, C. quinquefasciatus, C. rajah, C. restuans, C. salinarius, C. tarsalis, C. territans, C. theileri and C. tritaeniorhynchus; and Culiseta species e.g. Culiseta incidens, Culiseta impatiens, Culiseta inornata and Culiseta particeps.

According to one embodiment, the mosquitoes are capable of transmitting disease-causing pathogens. The pathogens transmitted by mosquitoes include viruses, protozoa, worms and bacteria.

Non-limiting examples of viral pathogens which may be transmitted by mosquitoes include the arbovirus pathogens such as Alphaviruses pathogens (e.g. Eastern Equine encephalitis virus, Western Equine encephalitis virus, Venezuelan Equine encephalitis virus, Ross River virus, Sindbis Virus and Chikungunya virus), Flavivirus pathogens (e.g. Japanese Encephalitis virus, Murray Valley Encephalitis virus, West Nile Fever virus, Yellow Fever virus, Dengue Fever virus, St. Louis encephalitis virus, and Tick-borne encephalitis virus), Bunyavirus pathogens (e.g. La Crosse Encephalitis virus, Rift Valley Fever virus, and Colorado Tick Fever virus), Orthobunyavirus pathogens (e.g. Oropouche virus) and Orbivirus (e.g. Bluetongue disease virus).

Non-limiting examples of worm pathogens which may be transmitted by mosquitoes include nematodes e.g. filarial nematodes such as Wuchereria bancrofti, Brugia malayi, Brugia pahangi, Brugia timori and heartworm (Dirofilaria immitis).

Non-limiting examples of bacterial pathogens which may be transmitted by mosquitoes include gram negative and gram positive bacteria including Yersinia pestis, Borellia spp, Rickettsia spp, and Erwinia carotovora.

Non-limiting examples of protozoa pathogens which may be transmitted by mosquitoes include the Malaria parasite of the genus Plasmodium e.g. Plasmodium falciparum, Plasmodium vivax, Plasmodium ovale, Plasmodium malariae, Plasmodium berghei, Plasmodium gallinaceum, and Plasmodium knowlesi.

As used herein, the phrase “pathogenically infected mosquito” refers to a mosquito carrying a disease-causing pathogen. Typically the mosquito is infected with the pathogen (e.g. via a blood meal) and acts as a vector for the pathogen, enabling replication of the pathogen (e.g. in the mid gut and salivary glands of the mosquito) and transmission thereof into a host.

It will be appreciated that the mosquito of the invention may be a healthy mosquito not infected or not yet infected by a pathogen.

A “host” may be any animal upon which the mosquito feeds and/or to which a mosquito is capable of transmitting a disease-causing pathogen. Non-limiting examples of hosts are mammals such as humans, domesticated pets (e.g. dogs and cats), wild animals (e.g. monkeys, rodents and wild cats), livestock animals (e.g. sheep, pigs, cattle, and horses), avians such as poultry (e.g. chickens, turkeys and ducks) and other animals such as crustaceans (e.g. prawns and lobsters), snakes and turtles.

According to one embodiment, the mosquito comprises a female mosquito being capable of transmitting a disease to a mammalian organism. According to another embodiment the female mosquito is pathogenically infected.

Non-limiting examples of mosquitoes and the pathogens which they transmit include species of the genus Anopheles (e.g. Anopheles gambiae) which transmit malaria parasites as well as microfilariae, arboviruses (including encephalitis viruses) and some species also transmit Wuchereria bancrofti; species of the genus Culex (e.g. C. pipiens) which transmit West Nile virus, filariasis, Japanese encephalitis, St. Louis encephalitis and avian malaria; species of the genus Aedes (e.g. Aedes aegypti, Aedes albopictus and Aedes polynesiensis) which transmit nematode worm pathogens (e.g. heartworm (Dirofilaria immitis)), arbovirus pathogens such as Alphaviruses pathogens that cause diseases such as Eastern Equine encephalitis, Western Equine encephalitis, Venezuelan equine encephalitis and Chikungunya disease; Flavivirus pathogens that cause diseases such as Japanese encephalitis, Murray Valley Encephalitis, West Nile fever, Yellow fever, Dengue fever, and Bunyavirus pathogens that cause diseases such as LaCrosse encephalitis, Rift Valley Fever, and Colorado tick fever.

According to one embodiment, pathogens that may be transmitted by Aedes aegypti are Dengue virus, Yellow fever virus, Chikungunya virus and heartworm (Dirofilaria immitis).

According to one embodiment, pathogens that may be transmitted by Aedes albopictus include West Nile Virus, Yellow Fever virus, St. Louis Encephalitis virus, Dengue virus, and Chikungunya fever virus.

According to one embodiment, pathogens that may be transmitted by Anopheles gambiae include malaria parasites of the genus Plasmodium such as, but not limited to, Plasmodium falciparum, Plasmodium vivax, Plasmodium ovale, Plasmodium malariae, Plasmodium berghei, Plasmodium gallinaceum, and Plasmodium knowlesi.

In another embodiment, the invention provides a method of controlling (e.g. exterminating) a pathogenically infected mosquito.

It will be appreciated that the mosquito of the invention is less likely to transmit a pathogen compared to its wild-type counterpart, since a mosquito encountering a pathogen (e.g. virus, protozoa, bacteria, nematode) will be impaired or exterminated (i.e. killed).

In one embodiment, the mosquito has an enhanced susceptibility to a pathogen.

As used herein, the term “enhanced susceptibility” refers to a mosquito which is more susceptible to a pathogen by at least 10%, 20%, 30%, 40%, 50%, or more, say 60%, 70%, 80%, 90% or more even 100% as compared to wild type (i.e. control) mosquito not treated by the agents of the invention.

Enhancing susceptibility of a mosquito to a pathogen is achieved by downregulating an expression of at least one mosquito pathogen resistance gene product of the mosquito.

As used herein, the term “mosquito pathogen resistance gene” refers to an endogenous gene of the mosquito (naturally occurring within the mosquito) whose product is involved in the natural resistance of the mosquito to a pathogen or to its products (e.g. toxins). According to one embodiment, a mosquito pathogen resistance gene is part of the mosquito's innate immunity.

As used herein, the term “endogenous” refers to a gene originating from within the organism, e.g. mosquito.

As used herein, the phrase “gene product” refers to an RNA molecule or a protein.

According to one embodiment, the mosquito pathogen resistance gene product is one which is essential for mosquito viability upon encounter with a pathogen. Downregulation of such a gene product would typically result in death of the mosquito upon encounter with the pathogen.

Specifically, in the absence of mosquito resistance to a pathogen (e.g. as a result of lack in digestive enzymes, digestive processes, intracellular processes and/or immune system), upon encounter with a pathogen (e.g. within a blood meal), the pathogen will typically replicate and exert a severe infection in the mosquito (e.g. in the midgut and/or salivary glands of the mosquito) and will overcome the natural barriers typically found in mosquitoes [e.g. the midgut infection barrier (MIB), the midgut escape barrier (MEB) and the salivary gland infection barrier (SIB)]. Thus, in the absence of mosquito resistance, the pathogen titers are significantly increased, the midgut infection is significantly increased, pathogen dissemination rates and extrinsic incubation periods are shortened and consequently high mortality of the mosquitoes is evident approximately 2 hours to 21 days (e.g. 12 hours to 15 days, 1 to 15 days, 3 to 10 days, 4 to 7 days) postinfection.

Exemplary pathogen resistance gene products that may be downregulated according to this aspect of the present invention include, but are not limited to, RNA interference related genes, piRNA pathway related genes, immunity related genes, metabolism related genes, cytoskeleton related genes, cell membrane related genes, cell motility related genes, extracellular structure related genes, post-translational modification related genes, protein turnover related genes, chaperone related genes, signal transduction related genes, proteolysis related genes, oxidoreductase activity related genes, transcription related genes, translation related genes, diverse related genes, transport related genes, cell-cycle related genes, energy production and conversion related genes, chromatin structure and dynamics related genes, Toll related genes and JAK/STAT related genes.

Tables 1A-B, below, provide a partial list of mosquito genes associated with pathogen resistance, which can be potential targets for reduction in expression by introducing the nucleic acid agent of the invention.

The present teachings contemplate the targeting of homologs and orthologs according to the selected mosquito species.

Homologous sequences include both orthologous and paralogous sequences. The term “paralogous” relates to gene-duplications within the genome of a species leading to paralogous genes. The term “orthologous” relates to homologous genes in different organisms due to ancestral relationship. Thus, orthologs are evolutionary counterparts derived from a single ancestral gene in the last common ancestor of given two species (Koonin E V and Galperin M Y (Sequence—Evolution—Function: Computational Approaches in Comparative Genomics. Boston: Kluwer Academic; 2003. Chapter 2, Evolutionary Concept in Genetics and Genomics. Available from: ncbi(dot)nlm(dot)nih(dot)gov/books/NBK20255) and therefore have great likelihood of having the same function. As such, orthologs usually play a similar role to that in the original species in another species.

Homology (e.g., percent homology, sequence identity+sequence similarity) can be determined using any homology comparison software computing a pairwise sequence alignment.

As used herein, “sequence identity” or “identity” in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. [Amino acid substitution matrices from protein blocks. Proc. Natl. Acad. Sci. U.S.A. 1992, 89(22): 10915-9].

According to a specific embodiment, the homolog sequences are at least 60%, 65%, 70%, 75%, 80%, 85%, 90%, 95% or even identical to the sequences (nucleic acid or amino acid sequences) provided hereinbelow.

TABLE 1A
List of target genes

| Aedes aegypti | Culex | Anopheles gambiae | |
|---|---|---|---|
| Access. No. | Access. No. | Access. No. | Functional group |
| TOLL pathway | | | |
| related genes | | | |
| AAEL009176 | CPIJ013556 | AGAP002796 | pattern recognition |
| (SEQ ID NO: 1), | (SEQ ID NO: 6), | (SEQ ID NO: 15), | receptor |
| AAEL003889 | CP1J008997 | AGAP012409 | |
| (SEQ ID NO: 2), | (SEQ ID NO: 7), | (SEQ ID NO: 16), | |
| AAEL009178 | CP1J004325 | AGAP004455 | |
| (SEQ ID NO: 3), | (SEQ ID NO: 8), | (SEQ ID NO: 17), | |
| AAEL007626 | CPIJ003613 | AGAP002799 | |
| (SEQ ID NO: 4), | (SEQ ID NO: 9), | (SEQ ID NO: 18), | |
| AAEL000652 | CP1J004324 | AGAP002798 | |
| (SEQ ID NO: 5) | (SEQ ID NO: 10), | (SEQ ID NO: 19), | |
| | CPIJ004323 | AGAP006761 | |
| | (SEQ ID NO: 11), | (SEQ ID NO: 20) | |
| | CPIJ004321 | | |
| | (SEQ ID NO: 12), | | |
| | CPIJ005217 | | |
| | (SEQ ID NO: 13), | | |
| | CPIJ004320 | | |
| | (SEQ ID NO: 14) | | |
| AAEL007064 | CPIJ004231 | | pattern recognition |
| (SEQ ID NO: 21) | (SEQ ID NO: 22) | | receptor |
| AAEL007347 | CPIJ001059 | AGAP011325 | signal modulation |
| (SEQ ID NO: 23), | (SEQ ID NO: 32), | (SEQ ID NO: 40), | |
| AAEL014724 | CP1J020127 | AGAP004855 | |
| (SEQ ID NO: 24), | (SEQ ID NO: 33), | (SEQ ID NO: 41) | |
| AAEL003243 | CPIJ003622 | | |
| (SEQ ID NO: 25), | (SEQ ID NO: 34), | | |
| AAEL003280 | CPIJ016225 | | |
| (SEQ ID NO: 26), | (SEQ ID NO: 35), | | |
| AAEL003233 | CPIJ017738 | | |
| (SEQ ID NO: 27), | (SEQ ID NO: 36), | | |
| AAEL003279 | CPIJ010296 | | |
| (SEQ ID NO: 28), | (SEQ ID NO: 37), | | |
| AAEL012775 | CPIJ010295 | | |
| (SEQ ID NO: 29), | (SEQ ID NO: 38), | | |
| AAEL014163 | CPIJ001060 | | |
| (SEQ ID NO: 30), | (SEQ ID NO: 39) | | |
| AAEL003253 | | | |
| (SEQ ID NO: 31) | | | |
| AAEL013435 | CPIJ000273 | AGAP006484 | Toll |
| (SEQ ID NO: 42), | (SEQ ID NO: 48), | (SEQ ID NO: 53), | |
| AAEL013434 | CP1J000272 | AGAP006483 | |
| (SEQ ID NO: 43), | (SEQ ID NO: 49), | (SEQ ID NO: 54), | |
| AAEL000499 | CP1J009906 | AGAP007177 | |
| (SEQ ID NO: 44), | (SEQ ID NO: 50), | (SEQ ID NO: 55) | |
| AAEL001435 | CPIJ006792 | | |
| (SEQ ID NO: 45), | (SEQ ID NO: 51), | | |
| AAEL013433 | CPIJ014270 | | |
| (SEQ ID NO: 46), | (SEQ ID NO: 52) | | |
| AAEL001929 | | | |
| (SEQ ID NO: 47) | | | |
| AAEL014989 | CPIJ008514 | AGAP005552 | pattern recognition |
| (SEQ ID NO: 56), | (SEQ ID NO: 58) | (SEQ ID NO: 59) | receptor |
| AAEL011608 | | | |
| (SEQ ID NO: 57) | | | |
| AAEL006930 | CPIJ015741 | AGAP009515 | Toll |
| (SEQ ID NO: 60), | (SEQ ID NO: 62), | (SEQ ID NO: 65) | |
| AAEL007696 | CPIJ019376 | | |
| (SEQ ID NO: 61) | (SEQ ID NO: 63), | | |
| | CPIJ002469 | | |
| | (SEQ ID NO: 64) | | |
| AAEL015515 | | | Effector |
| (SEQ ID NO: 66) | | | |
| AAEL004522 | CPIJ016084 | AGAP008645 | Effector |
| (SEQ ID NO: 67) | (SEQ ID NO: 68) | (SEQ ID NO: 69) | |
| AAEL003723 | CPIJ005451 | AGAP007347 | Effector |
| (SEQ ID NO: 70), | (SEQ ID NO: 76), | (SEQ ID NO: 81), | |
| AAEL009670 | CP1J005450 | AGAP005717 | |
| (SEQ ID NO: 71), | (SEQ ID NO: 77), | (SEQ ID NO: 82), | |
| AAEL015404 | CPIJ002161 | AGAP007386 | |
| (SEQ ID NO: 72), | (SEQ ID NO: 78), | (SEQ ID NO: 83), | |
| AAEL005988 | CPIJ010731 | AGAP007343 | |
| (SEQ ID NO: 73), | (SEQ ID NO: 79), | (SEQ ID NO: 84), | |
| AAEL003712 | CPIJ010730 | AGAP007385 | |
| (SEQ ID NO: 74), | (SEQ ID NO: 80) | (SEQ ID NO: 85) | |
| AAEL010100 | | | |
| (SEQ ID NO: 75) | | | |
| JAK/STAT | | | |
| pathway related | | | |
| genes | | | |
| AAEL012471 | CP1J017416 | AGAP010083 | |
| (SEQ ID NO: 86) | (SEQ ID NO: 87) | (SEQ ID NO: 88) | |
| AAEL012553 | CPIJ001760 | AGAP008354 | |
| (SEQ ID NO: 89) | (SEQ ID NO: 90) | (SEQ ID NO: 91) | |
| AAEL009692 | CPIJ016471 | AGAP010423 | |
| (SEQ ID NO: 92) | (SEQ ID NO: 93), | (SEQ ID NO: 95), | |
| | CPIJ016469 | AGAP000099 | |
| | (SEQ ID NO: 94) | (SEQ ID NO: 96) | |
| AAEL006949 | | AGAP000880 | |
| (SEQ ID NO: 97), | | (SEQ ID NO: 100), | |
| AAEL006936 | | AGAP004844 | |
| (SEQ ID NO: 98), | | (SEQ ID NO: 101) | |
| AAEL000255 | | | |
| (SEQ ID NO: 99) | | | |
| AAEL000393 | CP1J003379 | AGAP011042 | |
| (SEQ ID NO: 102) | (SEQ ID NO: 103), | (SEQ ID NO: 105) | |
| | CPIJ003380 | | |
| | (SEQ ID NO: 104) | | |
| AAEL015099 | CP1J009163 | AGAP005031 | |
| (SEQ ID NO: 106) | (SEQ ID NO: 107) | (SEQ ID NO: 108) | |
| Signal | | | |
| Transduction | | | |
| related genes | | | |
| AAEL012230 | CPIJ005494 | | |
| (SEQ ID NO: 109), | (SEQ ID NO: 112) | | |
| AAEL015328 | | | |
| (SEQ ID NO: 110), | | | |
| AAEL012225 | | | |
| (SEQ ID NO: 111) | | | |
| RNAi/piRNA | | | |
| pathway related | | | |
| genes | | | |
| AAEL011753 | CP1J011746 | AGAP009887 | r2d2 |
| (SEQ ID NO: 113), | (SEQ ID NO: 117), | (SEQ ID NO: 120), | |
| AAEL007470 | CP1J000296 | | |
| (SEQ ID NO: 114), | (SEQ ID NO: 118), | AGAP009781 | |
| AAEL013721 | CPIJ004832 | (SEQ ID NO: 121) | |
| (SEQ ID NO: 115), | (SEQ ID NO: 119) | | |
| AAEL008687 | | | |
| (SEQ ID NO: 116), | | | |
| AAEL006794 | CPIJ010534 | AGAP012289 | dcr2 |
| (SEQ ID NO: 122) | (SEQ ID NO: 123) | (SEQ ID NO: 124) | |
| AAEL007823 | CPIJ005275 | AGAP008862 | PIWI |
| (SEQ ID NO: 125) | (SEQ ID NO: 126) | (SEQ ID NO: 127) | |
| AAEL013235 | CPIJ017541 | AGAP002829 | Spn-E |
| (SEQ ID NO: 128) | (SEQ ID NO: 129) | (SEQ ID NO: 130) | |
| Chromatin | | | |
| structure and | | | |
| dynamics related | | | |
| genes | | | |
| AAEL003833 | CPIJ012378 | AGAP012586 | histone H4 |
| (SEQ ID NO: 131), | (SEQ ID NO: 143), | (SEQ ID NO: 145), | |
| AAEL003863 | CP1J014768 | AGAP003909 | |
| (SEQ ID NO: 132), | (SEQ ID NO: 144) | (SEQ ID NO: 146), | |
| AAEL003823 | | AGAP005026 | |
| (SEQ ID NO: 133), | | (SEQ ID NO: 147), | |
| AAEL003846 | | AGAP012574 | |
| (SEQ ID NO: 134), | | (SEQ ID NO: 148), | |
| AAEL003838 | | AGAP012558 | |
| (SEQ ID NO: 135), | | (SEQ ID NO: 149), | |
| AAEL000517 | | AGAP005023 | |
| (SEQ ID NO: 136), | | (SEQ ID NO: 150), | |
| AAEL003814 | | AGAP012885 | |
| (SEQ ID NO: 137), | | (SEQ ID NO: 151), | |
| AAEL000501 | | AGAP010724 | |
| (SEQ ID NO: 138), | | (SEQ ID NO: 152) | |
| AAEL003689 | | | |
| (SEQ ID NO: 139), | | | |
| AAEL003673 | | | |
| (SEQ ID NO: 140), | | | |
| AAEL000490 | | | |
| (SEQ ID NO: 141), | | | |
| AAEL000513 | | | |
| (SEQ ID NO: 142) | | | |
| AAEL000497 | CPIJ015010 | AGAP012711 | histone H2 |
| (SEQ ID NO: 153), | (SEQ ID NO: 166), | (SEQ ID NO: 182), | |
| AAEL003669 | CPIJ014767 | AGAP003911 | |
| (SEQ ID NO: 154), | (SEQ ID NO: 167), | (SEQ ID NO: 183), | |
| AAEL003862 | CP1J012408 | AGAP012198 | |
| (SEQ ID NO: 155), | (SEQ ID NO: 168), | (SEQ ID NO: 184), | |
| AAEL000518 | CPIJ012399 | AGAP012895 | |
| (SEQ ID NO: 156), | (SEQ ID NO: 169), | (SEQ ID NO: 185), | |
| AAEL007925 | CPIJ012421 | AGAP003913 | |
| (SEQ ID NO: 157), | (SEQ ID NO: 170), | (SEQ ID NO: 186) | |
| AAEL000525 | CPIJ012390 | | |
| (SEQ ID NO: 158), | (SEQ ID NO: 171), | | |
| AAEL000494 | CPIJ012412 | | |
| (SEQ ID NO: 159), | (SEQ ID NO: 172), | | |
| AAEL003706 | CPIJ010360 | | |
| (SEQ ID NO: 160), | (SEQ ID NO: 173), | | |
| AAEL003851 | CPIJ012419 | | |
| (SEQ ID NO: 161), | (SEQ ID NO: 174), | | |
| AAEL003820 | CPIJ005867 | | |
| (SEQ ID NO: 162), | (SEQ ID NO: 175), | | |
| AAEL003687 | CPIJ012380 | | |
| (SEQ ID NO: 163), | (SEQ ID NO: 176), | | |
| AAEL003826 | CPIJ012403 | | |
| (SEQ ID NO: 164), | (SEQ ID NO: 177), | | |
| AAEL003818 | CPIJ015004 | | |
| (SEQ ID NO: 165) | (SEQ ID NO: 178), | | |
| | CPIJ012395 | | |
| | (SEQ ID NO: 179), | | |
| | CPIJ004104 | | |
| | (SEQ ID NO: 180), | | |
| | CPIJ012425 | | |
| | (SEQ ID NO: 181) | | |
| AAEL003950 | CPIJ011033 | AGAP010699 | helicase |
| (SEQ ID NO: 187) | (SEQ ID NO: 188) | (SEQ ID NO: 189) | |
| Metabolism | | | |
| related genes | | | |
| AAEL008418 | CPIJ005082 | AGAP002308 | pyrroline-5-carboxylate |
| (SEQ ID NO: 190) | (SEQ ID NO: 191) | (SEQ ID NO: 192) | reductase |
| Cytoskeleton | | | |
| related genes | | | |
| AAEL000335 | CPIJ003869 | AGAP008015 | lamin |
| (SEQ ID NO: 193) | (SEQ ID NO: 194) | (SEQ ID NO: 195) | |
| Proteolysis | | | |
| related genes | | | |
| AAEL002610 | CPIJ017789 | AGAP011792 | serine protease |
| (SEQ ID NO: 196), | (SEQ ID NO: 201), | (SEQ ID NO: 204), | |
| AAEL002593 | CP1J017793 | AGAP011783 | |
| (SEQ ID NO: 197), | (SEQ ID NO: 202), | (SEQ ID NO: 205), | |
| AAEL002600 | CPIJ017792 | AGAP011789 | |
| (SEQ ID NO: 198), | (SEQ ID NO: 203) | (SEQ ID NO: 206), | |
| AAEL002629 | | AGAP011781 | |
| (SEQ ID NO: 199), | | (SEQ ID NO: 207), | |
| AAEL002595 | | AGAP011780 | |
| (SEQ ID NO: 200) | | (SEQ ID NO: 208), | |
| | | AGAP011787 | |
| | | (SEQ ID NO: 209), | |
| | | AGAP011793 | |
| | | (SEQ ID NO: 210) | |
| AAEL013857 | CP1J004497 | AGAP004700 | |
| (SEQ ID NO: 211) | (SEQ ID NO: 212) | (SEQ ID NO: 213) | |
| transcription and | | | |
| translation | | | |
| related genes | | | |
| AAEL005004 | CPIJ009211 | | hypothetical protein |
| (SEQ ID NO: 214) | (SEQ ID NO: 215) | | AaeL |
| AAEL005226 | CP1J015541 | AGAP005819 | |
| (SEQ ID NO: 216), | (SEQ ID NO: 234), | (SEQ ID NO: 259), | |
| AAEL008917 | CPIJ007121 | AGAP006214 | |
| (SEQ ID NO: 217), | (SEQ ID NO: 235), | (SEQ ID NO: 260), | |
| AAEL009082 | CP1J000335 | AGAP009638 | |
| (SEQ ID NO: 218), | (SEQ ID NO: 236), | (SEQ ID NO: 261), | |
| AAEL009592 | CP1J008475 | AGAP000906 | |
| (SEQ ID NO: 219), | (SEQ ID NO: 237), | (SEQ ID NO: 262), | |
| AAEL012429 | CP1J018334 | AGAP011037 | |
| (SEQ ID NO: 220), | (SEQ ID NO: 238), | (SEQ ID NO: 263), | |
| AAEL000710 | CPIJ016494 | AGAP002907 | |
| (SEQ ID NO: 221), | (SEQ ID NO: 239), | (SEQ ID NO: 264), | |
| AAEL000021 | CP1J019878 | AGAP000904 | |
| (SEQ ID NO: 222), | (SEQ ID NO: 240), | (SEQ ID NO: 265) | |
| AAEL003589 | CPIJ020103 | | |
| (SEQ ID NO: 223), | (SEQ ID NO: 241), | | |
| AAEL011208 | CPIJ013244 | | |
| (SEQ ID NO: 224), | (SEQ ID NO: 242), | | |
| AAEL009079 | CPIJ011969 | | |
| (SEQ ID NO: 225), | (SEQ ID NO: 243), | | |
| AAEL009063 | CPIJ005309 | | |
| (SEQ ID NO: 226), | (SEQ ID NO: 244), | | |
| AAEL003603 | CPIJ019506 | | |
| (SEQ ID NO: 227), | (SEQ ID NO: 245), | | |
| AAEL007264 | CPIJ012797 | | |
| (SEQ ID NO: 228), | (SEQ ID NO: 246), | | |
| AAEL007465 | CPIJ015236 | | |
| (SEQ ID NO: 229), | (SEQ ID NO: 247), | | |
| AAEL000406 | CPIJ016969 | | |
| (SEQ ID NO: 230), | (SEQ ID NO: 248), | | |
| AAEL005940 | CPIJ002091 | | |
| (SEQ ID NO: 231), | (SEQ ID NO: 249), | | |
| AAEL015447 | CPIJ011518 | | |
| (SEQ ID NO: 232), | (SEQ ID NO: 250), | | |
| AAEL015509 | CPIJ015370 | | |
| (SEQ ID NO: 233) | (SEQ ID NO: 251), | | |
| | CPIJ003366 | | |
| | (SEQ ID NO: 252), | | |
| | CPIJ007027 | | |
| | (SEQ ID NO: 253), | | |
| | CPIJ020171 | | |
| | (SEQ ID NO: 254), | | |
| | CPIJ006503 | | |
| | (SEQ ID NO: 255), | | |
| | CPIJ001232 | | |
| | (SEQ ID NO: 256), | | |
| | CPIJ015841 | | |
| | (SEQ ID NO: 257), | | |
| | CPIJ019509 | | |
| | (SEQ ID NO: 258) | | |
| AAEL002422 | CPIJ009745 | AGAP003080 | cytoplasmic |
| (SEQ ID NO: 266), | (SEQ ID NO: 268), | (SEQ ID NO: 270) | polyadenylation element |
| AAEL012065 | CPIJ009440 | | binding protein |
| (SEQ ID NO: 267) | (SEQ ID NO: 269) | | |
| Immunity related | | | |
| genes | | | |
| AAEL011455 | | | galactose-specific C- |
| (SEQ ID NO: 271) | | | type lectin |
| AAEL007599 | CPIJ015762 | AGAP004531 | cathepsin B |
| (SEQ ID NO: 272), | (SEQ ID NO: 277), | (SEQ ID NO: 279) | |
| AAEL007585 | CPIJ015761 | | |
| (SEQ ID NO: 273), | (SEQ ID NO: 278) | | |
| AAEL007590 | | | |
| (SEQ ID NO: 274), | | | |
| AAEL015312 | | | |
| (SEQ ID NO: 275), | | | |
| AAEL012216 | | | |
| (SEQ ID NO: 276) | | | |
| AAEL007585 | CPIJ015762 | AGAP004531 | cathepsin B |
| (SEQ ID NO: 280), | (SEQ ID NO: 285), | (SEQ ID NO: 287) | |
| AAEL007599 | CPIJ015761 | | |
| (SEQ ID NO: 281), | (SEQ ID NO: 286) | | |
| AAEL007590 | | | |
| (SEQ ID NO: 282), | | | |
| AAEL015312 | | | |
| (SEQ ID NO: 283), | | | |
| AAEL012216 | | | |
| (SEQ ID NO: 284) | | | |
| AAEL007585 | CPIJ015762 | AGAP004531 | cathepsin B |
| (SEQ ID NO: 288), | (SEQ ID NO: 293), | (SEQ ID NO: 295) | |
| AAEL007599 | CPIJ015761 | | |
| (SEQ ID NO: 289), | (SEQ ID NO: 294) | | |
| AAEL007590 | | | |
| (SEQ ID NO: 290), | | | |
| AAEL015312 | | | |
| (SEQ ID NO: 291), | | | |
| AAEL012216 | | | |
| (SEQ ID NO: 292) | | | |
| Redox activity | | | |
| related genes | | | |
| AAEL003211 | CPIJ001081 | AGAP008143 | beta-carotene |
| (SEQ ID NO: 296) | (SEQ ID NO: 297) | (SEQ ID NO: 298) | dioxygenase |
| Additional genes | | | |
| AAEL001702 | CP1J015159 | AGAP005312 | |
| (SEQ ID NO: 299) | (SEQ ID NO: 300) | (SEQ ID NO: 301) | |

TABLE 1B
List of Aedes aegypti target genes

| | Aedes aegypti target genes | |
|---|---|---|
| eq id no | Gene symbol | Gene Name |
| 305 | AAEL001411 | myosin heavy chain, nonmuscle or smooth muscle |
| 306 | AAEL014394 | growth factor receptor-bound protein |
| 307 | AAEL000700 | cadherin |
| 308 | AAEL001028 | hypothetical protein |
| 309 | AAEL010410 | odorant receptor 9a, putative |
| 310 | AAEL011202 | bhlhzip transcription factor bigmax |
| 311 | AAEL003355 | conserved hypothetical protein |
| 312 | AAEL002920 | hypothetical protein |
| 313 | AAEL012339 | cdk1 |
| 314 | AAEL013329 | cdk1 |
| 315 | AAEL009962 | hypothetical protein |
| 316 | AAEL000931 | alkaline phosphatase |
| 317 | AAEL000776 | conserved hypothetical protein |
| 318 | AAEL009022 | adenylate cyclase type |
| 319 | AAEL005766 | fructose-bisphosphate aldolase |
| 320 | AAEL002473 | hypothetical protein |
| 321 | AAEL012551 | conserved hypothetical protein |
| 322 | AAEL011648 | cyclin d |
| 323 | AAEL001246 | Thymidylate kinase, putative |
| 324 | AAEL011892 | receptor for activated C kinase, putative |
| 325 | AAEL003581 | amidophosphoribosyltransferase |
| 326 | AAEL014001 | yellow protein precursor, putative |
| 327 | AAEL012865 | conserved hypothetical protein |
| 328 | AAEL002510 | serine hydroxymethyltransferase |
| 329 | AAEL014025 | cell division cycle 20 (cdc20) (fizzy) |
| 330 | AAEL011250 | conserved hypothetical protein |
| 331 | AAEL010818 | hypothetical protein |
| 332 | AAEL005522 | conserved hypothetical protein |
| 333 | AAEL003325 | niemann-pick C1 |
| 334 | AAEL009773 | geminin, putative |
| 335 | AAEL004710 | spingomyelin synthetase |
| 336 | AAEL003465 | hypothetical protein |
| 337 | AAEL012510 | IMD pathway signaling I-Kappa-B Kinase 2 (IKK2 IKK-gamma). |
| 338 | AAEL013749 | conserved hypothetical protein |
| 339 | AAEL012085 | hypothetical protein |
| 340 | AAEL015080 | conserved hypothetical protein |
| 341 | AAEL013320 | translocon-associated protein, delta subunit |
| 342 | AAEL008686 | hypothetical protein |
| 343 | AAEL000217 | serine/threonine protein kinase |
| 344 | AAEL007799 | regulator of chromosome condensation |
| 345 | AAEL013912 | conserved hypothetical protein |
| 346 | AAEL002388 | zinc finger protein |
| 347 | AAEL012224 | zinc finger protein |
| 348 | AAEL010899 | hypothetical protein |
| 349 | AAEL010430 | ras-related protein, putative |
| 350 | AAEL003650 | inhibitor of growth protein, ing1 |
| 351 | AAEL005631 | conserved hypothetical protein |
| 352 | AAEL011295 | conserved hypothetical protein |
| 353 | AAEL003606 | purine biosynthesis protein 6, pur6 |
| 354 | AAEL010762 | Actin-related protein 8 |
| 355 | AAEL009645 | hypothetical protein |
| 356 | AAEL004699 | conserved hypothetical protein |
| 357 | AAEL012356 | GPCR Somatostatin Family |
| 358 | AAEL008084 | phosphatidylserine receptor |
| 359 | AAEL001352 | scaffold attachment factor b |
| 360 | AAEL007848 | conserved hypothetical protein |
| 361 | AAEL014844 | conserved hypothetical protein |
| 362 | AAEL002495 | conserved hypothetical protein |
| 363 | AAEL011714 | conserved hypothetical protein |
| 364 | AAEL008952 | sentrin/sumo-specific protease |
| 365 | AAEL011141 | hypothetical protein |
| 366 | AAEL010905 | conserved hypothetical protein |
| 367 | AAEL013797 | conserved hypothetical protein |
| 368 | AAEL007526 | electron transfer flavoprotein-ubiquinone oxidoreductase |
| 369 | AAEL006832 | GPCR Frizzled/Smoothened Family |
| 370 | AAEL011069 | conserved hypothetical protein |
| 371 | AAEL006519 | conserved hypothetical protein |
| 372 | AAEL012635 | conserved hypothetical protein |
| 373 | AAEL010659 | lethal(2)essential for life protein, l2efl |
| 374 | AAEL013343 | lethal(2)essential for life protein, l2efl |
| 375 | AAEL011639 | WAP four-disulfide core domain protein 2 precursor, putative |
| 376 | AAEL005439 | mical |
| 377 | AAEL000236 | hypothetical protein |
| 378 | AAEL012566 | conserved hypothetical protein |
| 379 | AAEL002896 | conserved hypothetical protein |
| 380 | AAEL006649 | tnf receptor associated factor |
| 381 | AAEL001856 | adenosine kinase |
| 382 | AAEL003549 | hypothetical protein |
| 383 | AAEL012043 | secreted modular calcium-binding protein |
| 384 | AAEL003425 | conserved hypothetical protein |
| 385 | AAEL007832 | GPCR Muscarinic Acetylcholine Family |
| 386 | AAEL015037 | G-protein-linked acetylcholine receptor gar-2a |
| 387 | AAEL001420 | leucine-rich immune protein (Short) |
| 388 | AAEL009615 | ultraviolet wavelength sensitive opsin |
| 389 | AAEL007397 | Ecdysone-induced protein 75B isoform A Nuclear receptor |
| 390 | AAEL000153 | conserved hypothetical protein |
| 391 | AAEL008015 | hypothetical protein |
| 392 | AAEL013552 | conserved hypothetical protein |
| 393 | AAEL005083 | conserved hypothetical protein |
| 394 | AAEL012562 | circadian locomoter output cycles kaput protein (dclock) (dpas1) |
| 395 | AAEL000580 | conserved hypothetical protein |
| 396 | AAEL011417 | synaptojanin |
| 397 | AAEL000041 | forkhead box protein (AaegFOXM2) |
| 398 | AAEL000945 | conserved hypothetical protein |
| 399 | AAEL002355 | conserved hypothetical protein |
| 400 | AAEL009230 | conserved hypothetical protein |
| 401 | AAEL002653 | semaphorin |
| 402 | AAEL009305 | numb-associated kinase |
| 403 | AAEL003574 | hypothetical protein |
| 404 | AAEL013040 | hypothetical protein |
| 405 | AAEL002400 | hypothetical protein |
| 406 | AAEL009382 | lysine-specific demethylase NO66 |
| | | (EC 1.14.11.27)(Nucleolar protein 66) |
| 407 | AAEL008320 | conserved hypothetical protein |
| 408 | AAEL001667 | multicopper oxidase |
| 409 | AAEL007073 | hypothetical protein |
| 410 | AAEL003152 | werner syndrome helicase |
| 411 | AAEL015522 | conserved hypothetical protein |
| 412 | AAEL014368 | sap18 |
| 413 | AAEL004607 | Adenylyltransferase and sulfurtransferase MOCS3 |
| | | (Molybdenum cofactor synthesis protein 3) |
| | | [Includes Adenylyltransferase MOCS3(EC 2.7.7.—) |
| | | (Sulfur carrier protein MOCS2A |
| 414 | AAEL001073 | malic enzyme |
| 415 | AAEL006087 | conserved hypothetical protein |
| 416 | AAEL006925 | conserved hypothetical protein |
| 417 | AAEL015285 | conserved hypothetical protein |
| 418 | AAEL010576 | modifier of mdg4 |
| 419 | AAEL011995 | conserved hypothetical protein |
| 420 | AAEL002064 | conserved hypothetical protein |
| 421 | AAEL009589 | conserved hypothetical protein |
| 422 | AAEL000356 | cysteine-rich venom protein, putative |
| 423 | AAEL000503 | hypothetical protein |
| 424 | AAEL012920 | GPCR Galanin/Allatostatin Family |
| 425 | AAEL014002 | conserved hypothetical protein |
| 426 | AAEL005850 | Hormone receptor-like in 4 (nuclear receptor) |
| 427 | AAEL000102 | conserved hypothetical protein |
| 428 | AAEL011647 | paired box protein, putative |
| 429 | AAEL005381 | Dissatisfaction (Dsf) |
| 430 | AAEL009360 | serine/threonine-protein kinase PLK4 |
| | | (EC 2.7.11.21)(Polo-like kinase 4)(PLK-4) |
| | | (Serine/threonine-protein kinase SAK) |
| 431 | AAEL012105 | Zinc finger protein-like 1 homolog |
| 432 | AAEL007053 | hypothetical protein |
| 433 | AAEL009822 | GPCR Metabotropic glutamate Family |
| 434 | AAEL013175 | hypothetical protein |
| 435 | AAEL009531 | niemann-pick C1 |
| 436 | AAEL009841 | conserved hypothetical protein |
| 437 | AAEL010333 | conserved hypothetical protein |
| 438 | AAEL005627 | chordin |
| 439 | AAEL001526 | zinc finger protein |
| 440 | AAEL007408 | conserved hypothetical protein |
| 441 | AAEL013280 | rho guanine exchange factor |
| 442 | AAEL009508 | zinc finger protein |
| 443 | AAEL008839 | hypothetical protein |
| 444 | AAEL015216 | serine/threonine-protein kinase vrk |
| 445 | AAEL007436 | conserved hypothetical protein |
| 446 | AAEL014392 | hypothetical protein |
| 447 | AAEL004458 | Zinc finger CCCH-type with G patch domain-containing |
| | | protein |
| 448 | AAEL000087 | macroglobulin/complement |
| 449 | AAEL000256 | Class B Scavenger Receptor (CD36 domain). |
| 450 | AAEL000274 | Copper-Zinc(Cu—Zn) Superoxide Dismutase. |
| 451 | AAEL000709 | TOLL pathway signaling. |
| 452 | AAEL000765 | hexamerin 2 beta |
| 453 | AAEL001794 | macroglobulin/complement |
| 454 | AAEL002585 | serine protease |
| 455 | AAEL002595 | serine protease |
| 456 | AAEL002629 | serine protease |
| 457 | AAEL002730 | Serine Protease Inhibitor (serpin) likely cleavage at R/V. |
| 458 | AAEL003119 | C-Type Lectin (CTL). |
| 459 | AAEL003439 | Caspase (Short). |
| 460 | AAEL003849 | defensin anti-microbial peptide |
| 461 | AAEL004386 | heme peroxidase |
| 462 | AAEL004388 | heme peroxidase |
| 463 | AAEL004390 | heme peroxidase |
| 464 | AAEL005064 | Clip-Domain Serine Protease family B. |
| 465 | AAEL005325 | dopachrome-conversion enzyme (DCE) isoenzyme, putative |
| 466 | AAEL005443 | conserved hypothetical protein |
| 467 | AAEL005673 | Serine Protease Inhibitor (serpin) likely cleavage at K/F. |
| 468 | AAEL005738 | yellow protein precursor |
| 469 | AAEL005832 | programmed cell death |
| 470 | AAEL006271 | copper-zinc (Cu—Zn) superoxide dismutase |
| 471 | AAEL006383 | chymotrypsin, putative |
| 472 | AAEL006576 | clip-domain serine protease, putative |
| 473 | AAEL006702 | fibrinogen and fibronectin |
| 474 | AAEL008364 | Serine Protease Inhibitor (serpin) likely cleavage at S/S. |
| 475 | AAEL009436 | conserved hypothetical protein |
| 476 | AAEL009861 | conserved hypothetical protein |
| 477 | AAEL010973 | conserved hypothetical protein |
| 478 | AAEL011498 | copper-zinc (Cu—Zn) superoxide dismutase |
| 479 | AAEL011699 | hypothetical protein |
| 480 | AAEL012267 | macroglobulin/complement |
| 481 | AAEL012958 | conserved hypothetical protein |
| 482 | AAEL013441 | Toll-like receptor |
| 483 | AAEL013757 | hexamerin 2 beta |
| 484 | AAEL013936 | Serine Protease Inhibitor (serpin) likely cleavage at I/S. |
| | | Transcript A. |
| 485 | AAEL014078 | serine protease inhibitor, serpin |
| 486 | AAEL014079 | serine protease inhibitor, serpin |
| 487 | AAEL014238 | aromatic amino acid decarboxylase |
| 488 | AAEL014390 | galactose-specific C-type lectin, putative |
| 489 | AAEL014548 | Thioredoxin Peroxidase. |
| 490 | AAEL014755 | tep2 |
| 491 | AAEL014989 | peptidoglycan recognition protein-1, putative |
| 492 | AAEL015322 | slit protein |
| 493 | AAEL007097 | 4-nitrophenylphosphatase |
| 494 | AAEL007323 | deoxyuridine 5′-triphosphate nucleotidohydrolase |
| 495 | AAEL006239 | glycerol kinase |
| 496 | AAEL002542 | triosephosphate isomerase |
| 497 | AAEL010208 | 3-hydroxyisobutyrate dehydrogenase |
| 498 | AAEL000006 | phosphoenolpyruvate carboxykinase |
| 499 | AAEL009245 | 3-hydroxyisobutyrate dehydrogenase, putative |
| 500 | AAEL015143 | glycine rich RNA binding protein, putative |
| 501 | AAEL006684 | Putative oxidoreductase GLYR1 homolog |
| | | (EC 1.—.—.—)(Glyoxylate reductase 1 homolog) |
| | | (Nuclear protein NP60 homolog) |
| 502 | AAEL012580 | 3-hydroxyisobutyrate dehydrogenase |
| 503 | AAEL013819 | Bj1 protein, putative |
| 504 | AAEL008849 | selenophosphate synthase |
| 505 | AAEL003084 | dolichyl-phosphate beta-D-mannosyltransferase, putative |
| 506 | AAEL014186 | dolichyl-phosphate beta-D-mannosyltransferase, putative |
| 507 | AAEL010751 | methylenetetrahydrofolate dehydrogenase |
| 508 | AAEL013877 | Glucosamine-6-phosphate isomerase |
| | | (EC 3.5.99.6)(Glucosamine-6-phosphate deaminase) |
| | | (GlcN6P deaminase)(GNPDA) |
| 509 | AAEL008166 | malate dehydrogenase |
| 510 | AAEL009721 | paraplegin |
| 511 | AAEL012337 | goliath E3 ubiquitin ligase |
| 512 | AAEL007593 | Clip-Domain Serine Protease family C. |
| 513 | AAEL003769 | methionine aminopeptidase |
| 514 | AAEL008416 | pre-mRNA processing factor |
| 515 | AAEL005201 | hydroxymethylglutaryl-coa synthase |
| 516 | AAEL008905 | host cell factor C1 |
| 517 | AAEL001112 | conserved hypothetical protein |
| 518 | AAEL002655 | matrix metalloproteinase |
| 519 | AAEL006323 | hypothetical protein |
| 520 | AAEL007649 | cell cycle checkpoint protein rad17 |
| 521 | AAEL004589 | small calcium-binding mitochondrial carrier, putative |
| 522 | AAEL011704 | heat shock protein |
| 523 | AAEL001052 | heat shock protein, putative |
| 524 | AAEL006362 | mitochondrial solute carrier |
| 525 | AAEL010002 | mitochondrial import inner membrane translocase subunit |
| | | tim17 |
| 526 | AAEL015575 | mitochondrial import inner membrane translocase subunit |
| | | tim17 |
| 527 | AAEL005413 | mitochondrial ribosomal protein, S11, putative |
| 528 | AAEL009964 | conserved hypothetical protein |
| 529 | AAEL010673 | NADH dehydrogenase, putative |
| 530 | AAEL001615 | mitochondrial ribosomal protein, S18C, putative |
| 531 | AAEL003215 | heat shock factor binding protein, putative |
| 532 | AAEL012499 | histone H2A |
| 533 | AAEL008500 | DEAD box ATP-dependent RNA helicase |
| 534 | AAEL007609 | histone H2A |
| 535 | AAEL005114 | RNA and export factor binding protein |
| 536 | AAEL015263 | RNA and export factor binding protein |
| 537 | AAEL006473 | arginine/serine-rich splicing factor |
| 538 | AAEL007928 | eukaryotic translation initiation factor 4 gamma |
| 539 | AAEL010340 | serine/arginine rich splicing factor |
| 540 | AAEL010402 | DEAD box ATP-dependent RNA helicase |
| 541 | AAEL003401 | DNA-directed RNA polymerase II 19 kDa polypeptide rpb7 |
| 542 | AAEL006135 | Nuclear cap-binding protein subunit 2 (20 kDa nuclear |
| | | cap-binding protein)(NCBP 20 kDa subunit)(CBP20) |
| 543 | AAEL009913 | DEAD box ATP-dependent RNA helicase |
| 544 | AAEL007078 | Eukaryotic translation initiation factor 3 subunit A |
| | | (eIF3a)(Eukaryotic translation initiation factor 3 subunit 10) |
| 545 | AAEL007923 | eukaryotic translation initiation factor 4 gamma |
| 546 | AAEL010612 | alternative splicing type 3 and, putative |
| 547 | AAEL011687 | alternative splicing type 3 and, putative |
| 548 | AAEL003893 | DNA repair protein xp-c/rad4 |
| 549 | AAEL006883 | conserved hypothetical protein |
| 550 | AAEL012585 | 60S ribosomal protein L7 |
| 551 | AAEL014429 | T-box transcription factor tbx20 |
| 552 | AAEL000098 | hypothetical protein |
| 553 | AAEL004174 | T-box transcription factor tbx6 |
| 554 | AAEL007458 | amino acid transporter |
| 555 | AAEL011470 | cis,cis-muconate transport protein MucK, putative |
| 556 | AAEL013146 | mfs transporter |
| 557 | AAEL002525 | amino acids transporter |
| 558 | AAEL006879 | folate carrier protein |
| 559 | AAEL012183 | mfs transporter |
| 560 | AAEL008878 | diacylglycerol o-acyltransferase |
| 561 | AAEL001968 | zinc transporter |
| 562 | AAEL009362 | cationic amino acid transporter |
| 563 | AAEL008138 | ABC transporter |
| 564 | AAEL005635 | nucleoporin |
| 565 | AAEL011679 | ion channel nompc |
| 566 | AAEL009421 | cyclophilin-r |
| 567 | AAEL003433 | copper-transporting ATPase 1, 2 (copper pump 1, 2) |
| 568 | AAEL006526 | neurotransmitter gated ion channel |
| 569 | AAEL004268 | Sialin, Sodium/sialic acid cotransporter, putative |
| 570 | AAEL005991 | tricarboxylate transport protein |
| 571 | AAEL009206 | organic cation transporter |
| 572 | AAEL002756 | synaptotagmin-4, |
| 573 | AAEL001405 | clathrin coat assembly protein |
| 574 | AAEL000675 | hypothetical protein |
| 575 | AAEL000727 | hypothetical protein |
| 576 | AAEL000969 | hypothetical protein |
| 577 | AAEL002095 | conserved hypothetical protein |
| 578 | AAEL002803 | conserved hypothetical protein |
| 579 | AAEL002975 | hypothetical protein |
| 580 | AAEL002979 | conserved hypothetical protein |
| 581 | AAEL003089 | conserved hypothetical protein |
| 582 | AAEL003131 | conserved hypothetical protein |
| 583 | AAEL003316 | hypothetical protein |
| 584 | AAEL003430 | conserved hypothetical protein |
| 585 | AAEL004498 | hypothetical protein |
| 586 | AAEL004604 | hypothetical protein |
| 587 | AAEL004625 | conserved hypothetical protein |
| 588 | AAEL004734 | conserved hypothetical protein |
| 589 | AAEL004754 | hypothetical protein |
| 590 | AAEL004976 | conserved hypothetical protein |
| 591 | AAEL005121 | conserved hypothetical protein |
| 592 | AAEL005192 | hypothetical protein |
| 593 | AAEL005389 | conserved hypothetical protein |
| 594 | AAEL006001 | conserved hypothetical protein |
| 595 | AAEL006072 | hypothetical protein |
| 596 | AAEL006243 | hypothetical protein |
| 597 | AAEL006247 | conserved hypothetical protein |
| 598 | AAEL006502 | conserved hypothetical protein |
| 599 | AAEL006606 | hypothetical protein |
| 600 | AAEL006755 | conserved hypothetical protein |
| 601 | AAEL007744 | hypothetical protein |
| 602 | AAEL007940 | gustatory receptor Gr77 |
| 603 | AAEL008439 | conserved hypothetical protein |
| 604 | AAEL008492 | conserved hypothetical protein |
| 605 | AAEL008636 | conserved hypothetical protein |
| 606 | AAEL009070 | hypothetical protein |
| 607 | AAEL009082 | hypothetical protein |
| 608 | AAEL009247 | conserved hypothetical protein |
| 609 | AAEL009322 | hypothetical protein |
| 610 | AAEL009385 | hypothetical protein |
| 611 | AAEL009473 | conserved hypothetical protein |
| 612 | AAEL009565 | conserved hypothetical protein |
| 613 | AAEL010022 | hypothetical protein |
| 614 | AAEL010113 | conserved hypothetical protein |
| 615 | AAEL010155 | hypothetical protein |
| 616 | AAEL010407 | conserved hypothetical protein |
| 617 | AAEL010898 | conserved hypothetical protein |
| 618 | AAEL011737 | hypothetical protein |
| 619 | AAEL011771 | hypothetical protein |
| 620 | AAEL011826 | conserved hypothetical protein |
| 621 | AAEL011872 | conserved hypothetical protein |
| 622 | AAEL012058 | hypothetical protein |
| 623 | AAEL012504 | hypothetical protein |
| 624 | AAEL012742 | conserved hypothetical protein |
| 625 | AAEL012754 | hypothetical protein |
| 626 | AAEL013024 | hypothetical protein |
| 627 | AAEL013037 | conserved hypothetical protein |
| 628 | AAEL013169 | conserved hypothetical protein |
| 629 | AAEL013776 | predicted protein |
| 630 | AAEL013977 | conserved hypothetical protein |
| 631 | AAEL014126 | hypothetical protein |
| 632 | AAEL014294 | conserved hypothetical protein |
| 633 | AAEL014816 | hypothetical protein |
| 634 | AAEL015613 | hypothetical protein |
| 635 | AAEL015634 | conserved hypothetical protein |
| 636 | AAEL001411 | myosin heavy chain, nonmuscle or smooth muscle |
| 637 | AAEL013778 | F-actin capping protein alpha |
| 638 | AAEL010510 | conserved hypothetical protein |
| 639 | AAEL011154 | hypothetical protein |
| 640 | AAEL004936 | conserved hypothetical protein |
| 641 | AAEL010979 | growth factor receptor-bound protein |
| 642 | AAEL001477 | laminin alpha-1, 2 chain |
| 643 | AAEL001904 | arp2/3 |
| 644 | AAEL002771 | microtubule binding protein, putative |
| 645 | AAEL005845 | beta chain spectrin |
| 646 | AAEL013808 | fascin |
| 647 | AAEL004440 | tubulin-specific chaperone e |
| 648 | AAEL000700 | cadherin |
| 649 | AAEL002761 | tropomyosin invertebrate |
| 650 | AAEL004668 | septin |
| 651 | AAEL003027 | conserved hypothetical protein |
| 652 | AAEL002185 | cuticle protein, putative |
| 653 | AAEL009527 | conserved hypothetical protein |
| 654 | AAEL014483 | conserved hypothetical protein |
| 655 | AAEL006340 | conserved hypothetical protein |
| 656 | AAEL012207 | myosin light chain 1, |
| 657 | AAEL008185 | conserved hypothetical protein |
| 658 | AAEL000048 | gustatory receptor Gr4 |
| 659 | AAEL003593 | hypothetical protein |
| 660 | AAEL015071 | gustatory receptor 64a, putative |
| 661 | AAEL013882 | tkr |
| 662 | AAEL007653 | allantoinase |
| 663 | AAEL000820 | dimethylaniline monooxygenase |
| 664 | AAEL014301 | hypothetical protein |
| 665 | AAEL003989 | GTP-binding protein alpha subunit, gna |
| 666 | AAEL011384 | hypothetical protein |
| 667 | AAEL010674 | hypothetical protein |
| 668 | AAEL007401 | roundabout, putative |
| 669 | AAEL006619 | conserved hypothetical protein |
| 670 | AAEL011105 | adducin |
| 671 | AAEL003220 | hypothetical protein |
| 672 | AAEL013028 | zinc finger protein |
| 673 | AAEL010755 | hypothetical protein |
| 674 | AAEL011552 | hypothetical protein |
| 675 | AAEL010301 | conserved hypothetical protein |
| 676 | AAEL008027 | hypothetical protein |
| 677 | AAEL014991 | hypothetical protein |
| 678 | AAEL004710 | spingomyelin synthetase |
| 679 | AAEL000405 | odd Oz protein |
| 680 | AAEL014746 | o-linked n-acetylglucosamine transferase, ogt |
| 681 | AAEL004715 | b-cell translocation protein |
| 682 | AAEL009646 | conserved hypothetical protein |
| 683 | AAEL003623 | conserved hypothetical protein |
| 684 | AAEL014042 | protein phosphatase pp2a regulatory subunit b |
| 685 | AAEL009249 | coronin |
| 686 | AAEL004351 | casein kinase |
| 687 | AAEL008806 | testis development protein prtd |
| 688 | AAEL003470 | conserved hypothetical protein |
| 689 | AAEL001434 | coronin |
| 690 | AAEL013969 | conserved hypothetical protein |
| 691 | AAEL012915 | als2cr7 |
| 692 | AAEL003571 | factor for adipocyte differentiation |
| 693 | AAEL001946 | four and a half lim domains |
| 694 | AAEL005795 | conserved hypothetical protein |
| 695 | AAEL007705 | hect E3 ubiquitin ligase |
| 696 | AAEL002705 | nucleolar protein c7b |
| 697 | AAEL005241 | lateral signaling target protein 2 |
| 698 | AAEL001853 | rac-GTP binding protein |
| 699 | AAEL003698 | conserved hypothetical protein |
| 700 | AAEL008879 | Kynurenine 3-monooxygenase (EC 1.14.13.9)(Kynurenine |
| | | 3-hydroxylase) |
| 701 | AAEL004501 | s-adenosylmethionine synthetase |
| 702 | AAEL003145 | bestrophin 2,3,4 |
| 703 | AAEL006786 | GTPase_rho |
| 704 | AAEL008171 | double-stranded RNA-binding protein zn72d |
| 705 | AAEL008007 | conserved hypothetical protein |
| 706 | AAEL010665 | developmentally regulated RNA-binding protein |
| 707 | AAEL013057 | serine/threonine-protein kinase wnk 1,3,4 |
| 708 | AAEL002082 | latent nuclear antigen, putative |
| 709 | AAEL002090 | conserved hypothetical protein |
| 710 | AAEL004041 | flotillin-2 |
| 711 | AAEL010676 | regulator of g protein signaling |
| 712 | AAEL008739 | shc transforming protein |
| 713 | AAEL011061 | hypothetical protein |
| 714 | AAEL007479 | hypothetical protein |
| 715 | AAEL014851 | mediator complex subunit rgr-1 |
| 716 | AAEL005930 | ubiquitin-protein ligase |
| 717 | AAEL002277 | cAMP-dependent protein kinase type i-beta regulatory |
| | | subunit |
| 718 | AAEL009422 | conserved hypothetical protein |
| 719 | AAEL006460 | par-6 gamma |
| 720 | AAEL001848 | conserved hypothetical protein |
| 721 | AAEL002607 | conserved hypothetical protein |
| 722 | AAEL000090 | secretory carrier-associated membrane protein (scamp) |
| 723 | AAEL005535 | conserved hypothetical protein |
| 724 | AAEL010344 | SEC14, putative |
| 725 | AAEL011006 | Guanylate kinase |
| 726 | AAEL006539 | serine/threonine protein kinase |
| 727 | AAEL005284 | receptor tyrosine phosphatase type r2a |
| 728 | AAEL009495 | rab6-interacting |
| 729 | AAEL005400 | 2-hydroxyacid dehydrogenase |
| 730 | AAEL000395 | Ultra spiracleisoform A nuclear receptor |
| 731 | AAEL002175 | conserved hypothetical protein |
| 732 | AAEL010170 | ras-related protein Rab-8A, putative |
| 733 | AAEL007889 | F-spondin |
| 734 | AAEL008078 | clk2 |
| 735 | AAEL014510 | sprouty |
| 736 | AAEL011417 | synaptojanin |
| 737 | AAEL000591 | hypothetical protein |
| 738 | AAEL001528 | hypothetical protein |
| 739 | AAEL005369 | zinc finger protein |
| 740 | AAEL010668 | quinone oxidoreductase |
| 741 | AAEL001099 | DEAD box polypeptide |
| 742 | AAEL002451 | zinc finger protein |
| 743 | AAEL003845 | Ets domain-containing protein |
| 744 | AAEL011970 | GPCR Purine/Adenosine Family |
| 745 | AAEL007322 | phosphatidate phosphatase |
| 746 | AAEL010561 | conserved hypothetical protein |
| 747 | AAEL006780 | hypothetical protein |
| 748 | AAEL007436 | conserved hypothetical protein |
| 749 | AAEL000737 | rab6 GTPase activating protein, gapcena (rabgap1 protein) |
| 750 | AAEL001133 | conserved hypothetical protein |
| 751 | AAEL005683 | conserved hypothetical protein |
| 752 | AAEL007375 | pyruvate dehydrogenase |
| 753 | AAEL001393 | triple functional domain, trio |
| 754 | AAEL005238 | mck1 |
| 755 | AAEL009874 | conserved hypothetical protein |
| 756 | AAEL001375 | Y-box binding protein |
| 757 | AAEL013308 | odd Oz protein |
| 758 | AAEL001398 | guanine nucleotide exchange factor |
| 759 | AAEL009171 | conserved hypothetical protein |
| 760 | AAEL004964 | hypothetical protein |
| 761 | AAEL009264 | hypothetical protein |
| 762 | AAEL001898 | conserved hypothetical protein |
| 763 | AAEL000421 | protein farnesyltransferase alpha subunit/rab geranylgeranyl |
| | | transferase alpha subunit |
| 764 | AAEL012554 | maltose phosphorylase |
| 765 | AAEL000262 | conserved hypothetical protein |
| 766 | AAEL000770 | platelet-activating factor acetylhydrolase isoform 1b alpha |
| | | subunit |
| 767 | AAEL003976 | conserved hypothetical protein |
| 768 | AAEL002937 | hypothetical protein |
| 769 | AAEL003540 | conserved hypothetical protein |
| 770 | AAEL005706 | triacylglycerol lipase |
| 771 | AAEL007662 | casein kinase |
| 772 | AAEL013619 | dolichyl-diphosphooligosaccharide protein |
| | | glycosyltransferase |
| 773 | AAEL004209 | opioid-binding protein/cell adhesion molecule, putative |
| 774 | AAEL003750 | conserved hypothetical protein |
| 775 | AAEL004709 | protein phosphatase type 2c |
| 776 | AAEL009382 | lysine-specific demethylase NO66 |
| | | (EC 1.14.11.27)(Nucleolar protein 66) |
| 777 | AAEL014999 | conserved hypothetical protein |
| 778 | AAEL012076 | conserved hypothetical protein |
| 779 | AAEL013334 | conserved hypothetical protein |
| 780 | AAEL005861 | vacuolar sorting protein (vps) |
| 781 | AAEL002251 | conserved hypothetical protein |
| 782 | AAEL009645 | hypothetical protein |
| 783 | AAEL000713 | reticulon/nogo |
| 784 | AAEL006651 | dystrophin |
| 785 | AAEL009606 | conserved hypothetical protein |
| 786 | AAEL008591 | zinc finger protein, putative |
| 787 | AAEL013459 | conserved hypothetical protein |
| 788 | AAEL006041 | conserved hypothetical protein |
| 789 | AAEL013510 | smaug protein |
| 790 | AAEL005528 | conserved hypothetical protein |
| 791 | AAEL003824 | conserved hypothetical protein |
| 792 | AAEL011575 | conserved hypothetical protein |
| 793 | AAEL006990 | conserved hypothetical protein |
| 794 | AAEL002306 | hect E3 ubiquitin ligase |
| 795 | AAEL013068 | protein phsophatase-2a |
| 796 | AAEL005320 | skeletrophin |
| 797 | AAEL000079 | hypothetical protein |
| 798 | AAEL010020 | Mediator of RNA polymerase II transcription subunit 14 |
| | | (Mediator complex subunit 14) |
| 799 | AAEL007011 | conserved hypothetical protein |
| 800 | AAEL000399 | conserved hypothetical protein |
| 801 | AAEL001919 | protein tyrosine phosphatase, non-receptor type nt1 |
| 802 | AAEL005302 | beta-1,4-galactosyltransferase |
| 803 | AAEL003509 | smap1 |
| 804 | AAEL003955 | hypothetical protein |
| 805 | AAEL003928 | pdgf/vegf receptor |
| 806 | AAEL000824 | hypothetical protein |
| 807 | AAEL004472 | hypothetical protein |
| 808 | AAEL010750 | hypothetical protein |
| 809 | AAEL002706 | hypothetical protein |
| 810 | AAEL007884 | conserved membrane protein at 44E, putative |
| 811 | AAEL008107 | f14p3.9 protein (auxin transport protein) |
| 812 | AAEL000857 | conserved hypothetical protein |
| 813 | AAEL014931 | sarm1 |
| 814 | AAEL001709 | hypothetical protein |
| 815 | AAEL008733 | histidine triad (hit) protein member |
| 816 | AAEL005502 | conserved hypothetical protein |
| 817 | AAEL001640 | multicopper oxidase |
| 818 | AAEL003799 | autophagy related gene |
| 819 | AAEL002142 | conserved hypothetical protein |
| 820 | AAEL015466 | conserved hypothetical protein |
| 821 | AAEL007687 | transmembrane 9 superfamily protein member 4 |
| 822 | AAEL013280 | rho guanine exchange factor |
| 823 | AAEL003454 | phocein protein, putative |
| 824 | AAEL001152 | beta-1,3-galactosyltransferase-6 |
| 825 | AAEL008793 | conserved hypothetical protein |
| 826 | AAEL007455 | thrombospondin |
| 827 | AAEL013072 | conserved hypothetical protein |
| 828 | AAEL007370 | conserved hypothetical protein |
| 829 | AAEL002732 | nephrin |
| 830 | AAEL002364 | hypothetical protein |
| 831 | AAEL007665 | hypothetical protein |
| 832 | AAEL002637 | tripartite motif protein trim9 |
| 833 | AAEL011623 | conserved hypothetical protein |
| 834 | AAEL014622 | conserved hypothetical protein |
| 835 | AAEL015487 | zinc finger protein, putative |
| 836 | AAEL010229 | hypothetical protein |
| 837 | AAEL004412 | polo kinase kinase |
| 838 | AAEL002448 | hypothetical protein |
| 839 | AAEL001388 | hypothetical protein |
| 840 | AAEL012998 | conserved hypothetical protein |
| 841 | AAEL013231 | hypothetical protein |
| 842 | AAEL010062 | conserved hypothetical protein |
| 843 | AAEL007199 | hypothetical protein |
| 844 | AAEL005109 | WD-repeat protein |
| 845 | AAEL003312 | hypothetical protein |
| 846 | AAEL013430 | putative G-protein coupled receptor (GPCR) |
| 847 | AAEL003508 | serine-pyruvate aminotransferase |
| 848 | AAEL002120 | zinc finger protein |
| 849 | AAEL004508 | hypothetical protein |
| 850 | AAEL012570 | hypothetical protein |
| 851 | AAEL001569 | conserved hypothetical protein |
| 852 | AAEL001094 | conserved hypothetical protein |
| 853 | AAEL000165 | conserved hypothetical protein |
| 854 | AAEL012086 | leucine-rich immune protein (Long) |
| 855 | AAEL009520 | leucine-rich immune protein (Long) |
| 856 | AAEL000703 | glycogen phosphorylase |
| 857 | AAEL007677 | phospholysine phosphohistidine inorganic pyrophosphate |
| | | phosphatase |
| 858 | AAEL011220 | Ati or CPXV158 protein, putative |
| 859 | AAEL001635 | conserved hypothetical protein |
| 860 | AAEL000541 | fasciclin, putative |
| 861 | AAEL005216 | Conserved hypothetical protein |
| 862 | AAEL004221 | glycogen synthase |
| 863 | AAEL004150 | fibrinogen and fibronectin |
| 864 | AAEL012187 | lethal(3)malignant brain tumor |
| 865 | AAEL003651 | conserved hypothetical protein |
| 866 | AAEL003729 | Probable hydroxyacid-oxoacid transhydrogenase, |
| | | mitochondrial Precursor (HOT)(EC 1.1.99.24) |
| 867 | AAEL013453 | sarcolemmal associated protein, putative |
| 868 | AAEL001650 | conserved hypothetical protein |
| 869 | AAEL002569 | serine/threonine kinase |
| 870 | AAEL012238 | glutaredoxin, putative |
| 871 | AAEL004229 | glutathione transferase |
| 872 | AAEL011596 | mitotic checkpoint serine/threonine-protein kinase bub1 and |
| | | bubr1 |
| 873 | AAEL006207 | conserved hypothetical protein |
| 874 | AAEL014596 | hypothetical protein |
| 875 | AAEL012391 | conserved hypothetical protein |
| 876 | AAEL013974 | conserved hypothetical protein |
| 877 | AAEL008719 | Sm protein G, putative |
| 878 | AAEL008316 | mitotic spindle assembly checkpoint protein mad2 |
| 879 | AAEL008646 | fibrinogen and fibronectin |
| 880 | AAEL011235 | conserved hypothetical protein |
| 881 | AAEL008716 | conserved hypothetical protein |
| 882 | AAEL015555 | conserved hypothetical protein |
| 883 | AAEL012628 | conserved hypothetical protein |
| 884 | AAEL000465 | conserved hypothetical protein |
| 885 | AAEL008369 | acyl phosphatase, putative |
| 886 | AAEL004512 | zinc finger protein |
| 887 | AAEL005557 | hypothetical protein |
| 888 | AAEL001653 | fetal globin-inducing factor |
| 889 | AAEL010622 | hypothetical protein |
| 890 | AAEL007907 | serine/threonine protein kinase |
| 891 | AAEL010013 | WD-repeat protein |
| 892 | AAEL002739 | conserved hypothetical protein |
| 893 | AAEL011834 | hypothetical protein |
| 894 | AAEL000147 | single-stranded DNA binding protein, putative |
| 895 | AAEL013943 | mediator complex, 100 kD-subunit, putative |
| 896 | AAEL005976 | adenine phosphoribosyltransferase, putative |
| 897 | AAEL001838 | conserved hypothetical protein |
| 898 | AAEL000425 | conserved hypothetical protein |
| 899 | AAEL015060 | Rad51A protein, putative |
| 900 | AAEL015658 | conserved hypothetical protein |
| 901 | AAEL004086 | aldo-keto reductase |
| 902 | AAEL009701 | conserved hypothetical protein |
| 903 | AAEL011362 | hypothetical protein |
| 904 | AAEL007395 | conserved hypothetical protein |
| 905 | AAEL007564 | zinc finger protein |
| 906 | AAEL002888 | williams-beuren syndrome critical region protein |
| 907 | AAEL012771 | leucine-rich immune protein (Coil-less) |
| 908 | AAEL009149 | kinectin, putative |
| 909 | AAEL009425 | hypothetical protein |
| 910 | AAEL012938 | zinc finger protein |
| 911 | AAEL005719 | cleavage stimulation factor |
| 912 | AAEL013844 | diazepam binding inhibitor, putative |
| 913 | AAEL006787 | conserved hypothetical protein |
| 914 | AAEL006948 | tomosyn |
| 915 | AAEL004335 | secreted ferritin G subunit precursor, putative |
| 916 | AAEL014438 | juvenile hormone-inducible protein, putative |
| 917 | AAEL011606 | conserved hypothetical protein |
| 918 | AAEL008486 | protein kinase C inhibitor, putative |
| 919 | AAEL006628 | conserved hypothetical protein |
| 920 | AAEL000065 | conserved hypothetical protein |
| 921 | AAEL005297 | guanine nucleotide exchange factor |
| 922 | AAEL013338 | lethal(2)essential for life protein, l2efl |
| 923 | AAEL015636 | interleukin enhancer binding factor |
| 924 | AAEL010472 | helix-loop-helix protein hen |
| 925 | AAEL002950 | conserved hypothetical protein |
| 926 | AAEL005395 | conserved hypothetical protein |
| 927 | AAEL000629 | adenylate kinase 3, |
| 928 | AAEL004004 | chromatin regulatory protein sir2 |
| 929 | AAEL011816 | conserved hypothetical protein |
| 930 | AAEL002399 | aspartate aminotransferase |
| 931 | AAEL006203 | juvenile hormone-inducible protein, putative |
| 932 | AAEL015017 | islet cell autoantigen |
| 933 | AAEL013644 | ubiquitously transcribed sex (x/y) chromosome |
| | | tetratricopeptide repeat protein |
| 934 | AAEL006965 | NBP2b protein, putative |
| 935 | AAEL004566 | myo inositol monophosphatase |
| 936 | AAEL012939 | gamma-subunit,methylmalonyl-CoA decarboxylase, |
| | | putative |
| 937 | AAEL001703 | serine-type enodpeptidase, |
| 938 | AAEL002273 | trypsin, putative |
| 939 | AAEL010951 | glutamate decarboxylase |
| 940 | AAEL007363 | leucine-rich transmembrane protein |
| 941 | AAEL007613 | Toll-like receptor |
| 942 | AAEL002166 | leucine rich repeat (in flii) interacting protein |
| 943 | AAEL002206 | rap GTPase-activating protein |
| 944 | AAEL005832 | programmed cell death |
| 945 | AAEL000709 | TOLL pathway signaling. |
| 946 | AAEL003119 | C-Type Lectin (CTL). |
| 947 | AAEL014989 | peptidoglycan recognition protein-1, putative |
| 948 | AAEL014356 | C-Type Lectin (CTL) - selectin like. |
| 949 | AAEL003554 | leucine rich repeat protein |
| 950 | AAEL001914 | scavenger receptor, putative |
| 951 | AAEL006702 | fibrinogen and fibronectin |
| 952 | AAEL006699 | fibrinogen and fibronectin |
| 953 | AAEL011764 | prophenoloxidase |
| 954 | AAEL006137 | Serine Protease Inhibitor (serpin) homologue - |
| | | unlikely to be inhibitory. |
| 955 | AAEL009420 | Class B Scavenger Receptor (CD36 domain). |
| 956 | AAEL013417 | fibrinogen and fibronectin |
| 957 | AAEL000533 | C-Type Lectin (CTL). |
| 958 | AAEL002354 | heme peroxidase |
| 959 | AAEL002704 | Serine Protease Inhibitor (serpin) homologue |
| 960 | AAEL000633 | Toll-like receptor |
| 961 | AAEL008681 | C-Type Lectin (CTL). |
| 962 | AAEL009551 | Toll-like receptor |
| 963 | AAEL009176 | Gram-Negative Binding Protein (GNBP) or Beta-1 3- |
| | | Glucan Binding Protein (BGBP). |
| 964 | AAEL007768 | TOLL pathway signaling. |
| 965 | AAEL000227 | Class B Scavenger Receptor (CD36 domain). |
| 966 | AAEL001163 | macroglobulin/complement |
| 967 | AAEL009474 | Peptidoglycan Recognition Protein (Short) |
| 968 | AAEL011009 | fibrinogen and fibronectin |
| 969 | AAEL009384 | fibrinogen and fibronectin |
| 970 | AAEL005800 | Clip-Domain Serine Protease family E. Protease |
| | | homologue. |
| 971 | AAEL007107 | serine protease, putative |
| 972 | AAEL002601 | Clip-Domain Serine Protease family A. Protease |
| | | homologue. |
| 973 | AAEL007626 | Gram-Negative Binding Protein (GNBP) or Beta-1 3- |
| | | Glucan Binding Protein (BGBP). |
| 974 | AAEL003632 | Clip-Domain Serine Protease family B. |
| 975 | AAEL006161 | Clip-Domain Serine Protease family B. |
| 976 | AAEL003857 | defensin anti-microbial peptide |
| 977 | AAEL004868 | hemomucin |
| 978 | AAEL009842 | galectin |
| 979 | AAEL014246 | glucosyl/glucuronosyl transferases |
| 980 | AAEL002688 | glucosyl/glucuronosyl transferases |
| 981 | AAEL013128 | elongase, putative |
| 982 | AAEL014664 | AMP dependent coa ligase |
| 983 | AAEL001273 | Sec24B protein, putative |
| 984 | AAEL013458 | glutamine synthetase 1, 2 (glutamate- ammonia ligase) (gs) |
| 985 | AAEL010256 | E3 ubiquitin ligase |
| 986 | AAEL006687 | exportin |
| 987 | AAEL014871 | methylenetetrahydrofolate dehydrogenase |
| 988 | AAEL002430 | n-acetylglucosamine-6-phosphate deacetylase |
| 989 | AAEL010751 | methylenetetrahydrofolate dehydrogenase |
| 990 | AAEL004952 | protein N-terminal asparagine amidohydrolase, putative |
| 991 | AAEL008374 | E3 ubiquitin-protein ligase nedd-4 |
| 992 | AAEL008687 | tar RNA binding protein (trbp) |
| 993 | AAEL004294 | dihydrolipoamide acetyltransferase component |
| | | of pyruvate dehydrogenase |
| 994 | AAEL005763 | lysosomal alpha-mannosidase (mannosidase |
| | | alpha class 2b member 1) |
| 995 | AAEL008507 | srpk |
| 996 | AAEL001593 | glycerol-3-phosphate dehydrogenase |
| 997 | AAEL004865 | cyclin g |
| 998 | AAEL003402 | sphingomyelin phosphodiesterase |
| 999 | AAEL003091 | glucosyl/glucuronosyl transferases |
| 1000 | AAEL008393 | phosphatidylserine synthase |
| 1001 | AAEL001523 | secretory Phospholipase A2, putative |
| 1002 | AAEL014965 | nova |
| 1003 | AAEL005380 | mixed-lineage leukemia protein, mll |
| 1004 | AAEL003873 | glycerol-3-phosphate dehydrogenase |
| 1005 | AAEL004757 | cleavage and polyadenylation specificity factor |
| 1006 | AAEL002528 | histone deacetylase |
| 1007 | AAEL000690 | steroid dehydrogenase |
| 1008 | AAEL011957 | elongase, putative |
| 1009 | AAEL012446 | Inhibitor of Apoptosis (IAP) containing Baculoviral IAP |
| | | Repeat(s) (BIR domains). |
| 1010 | AAEL000006 | phosphoenolpyruvate carboxykinase |
| 1011 | AAEL013525 | Timp-3, putative |
| 1012 | AAEL002658 | AMP dependent ligase |
| 1013 | AAEL013831 | pyrroline-5-carboxylate dehydrogenase |
| 1014 | AAEL002542 | triosephosphate isomerase |
| 1015 | AAEL012014 | l-lactate dehydrogenase |
| 1016 | AAEL012418 | deoxyribonuclease ii |
| 1017 | AAEL009237 | glycoside hydrolases |
| 1018 | AAEL012994 | glucose-6-phosphate isomerase |
| 1019 | AAEL012455 | alcohol dehydrogenase |
| 1020 | AAEL015020 | glycoside hydrolases |
| 1021 | AAEL004778 | acyl-coa dehydrogenase |
| 1022 | AAEL008865 | oligoribonuclease, mitochondrial |
| 1023 | AAEL007893 | short chain type dehydrogenase |
| 1024 | AAEL014139 | proacrosin, putative |
| 1025 | AAEL008668 | Clip-Domain Serine Protease family B. |
| 1026 | AAEL008124 | possible RNA methyltransferase, putative |
| 1027 | AAEL014353 | conserved hypothetical protein |
| 1028 | AAEL003026 | regulator of g protein signaling |
| 1029 | AAEL002663 | kuzbanian |
| 1030 | AAEL008202 | serine-type enodpeptidase, |
| 1031 | AAEL004138 | signal peptide peptidase |
| 1032 | AAEL004980 | conserved hypothetical protein |
| 1033 | AAEL003733 | hypothetical protein |
| 1034 | AAEL001540 | ubiquitin specific protease |
| 1035 | AAEL003965 | calpain 4, 6, 7, invertebrate |
| 1036 | AAEL006542 | retinoid-inducible serine carboxypeptidase (serine |
| | | carboxypeptidase |
| 1037 | AAEL013605 | hypothetical protein |
| 1038 | AAEL005107 | hypothetical protein |
| 1039 | AAEL015272 | zinc carboxypeptidase |
| 1040 | AAEL008769 | serine-type enodpeptidase, |
| 1041 | AAEL003967 | calpain 4, 6, 7, invertebrate |
| 1042 | AAEL010989 | hypothetical protein |
| 1043 | AAEL005342 | conserved hypothetical protein |
| 1044 | AAEL011850 | cytochrome P450 |
| 1045 | AAEL006386 | mitochondrial 39S ribosomal protein L39 |
| 1046 | AAEL010226 | daughterless |
| 1047 | AAEL004589 | small calcium-binding mitochondrial carrier, putative |
| 1048 | AAEL014608 | cytochrome P450 |
| 1049 | AAEL007235 | mitochondrial uncoupling protein |
| 1050 | AAEL003215 | heat shock factor binding protein, putative |
| 1051 | AAEL010546 | heat shock factor binding protein, putative |
| 1052 | AAEL000895 | peroxisome biogenesis factor 1 (peroxin-1) |
| 1053 | AAEL001024 | mitochondrial carrier protein |
| 1054 | AAEL006318 | short-chain dehydrogenase |
| 1055 | AAEL013350 | heat shock protein 26 kD, putative |
| 1056 | AAEL007046 | mitochondrial brown fat uncoupling protein |
| 1057 | AAEL010372 | aldehyde oxidase |
| 1058 | AAEL013693 | excision repair cross-complementing 1 ercc1 |
| 1059 | AAEL012308 | hypothetical protein |
| 1060 | AAEL003195 | Carboxy/choline esterase Alpha Esterase |
| 1061 | AAEL010677 | oxidoreductase |
| 1062 | AAEL010380 | aldehyde oxidase |
| 1063 | AAEL002523 | mitochondrial inner membrane protein translocase, 9 kD- |
| | | subunit, putative |
| 1064 | AAEL002486 | mitochondrial inner membrane protein translocase, 9 kD- |
| | | subunit, putative |
| 1065 | AAEL004829 | NADH dehydrogenase, putative |
| 1066 | AAEL011752 | glutathione transferase |
| 1067 | AAEL006984 | cytochrome P450 |
| 1068 | AAEL007355 | mitochondrial ribosomal protein, S18A, putative |
| 1069 | AAEL003770 | conserved hypothetical protein |
| 1070 | AAEL002783 | mitochondrial ribosomal protein, L37, putative |
| 1071 | AAEL004450 | cytochrome b5, putative |
| 1072 | AAEL008601 | mitochondrial ribosomal protein, L28, putative |
| 1073 | AAEL007946 | glutathione transferase |
| 1074 | AAEL013790 | mitochondrial ribosomal protein, L50, putative |
| 1075 | AAEL005113 | Carboxy/choline esterase Alpha Esterase |
| 1076 | AAEL004716 | chromodomain helicase DNA binding protein |
| 1077 | AAEL007923 | eukaryotic translation initiation factor 4 gamma |
| 1078 | AAEL010467 | heterogeneous nuclear ribonucleoprotein |
| 1079 | AAEL004119 | ribonuclease p/mrp subunit |
| 1080 | AAEL013653 | tata-box binding protein |
| 1081 | AAEL010222 | transcription factor GATA-4 (GATA binding factor-4) |
| 1082 | AAEL015263 | RNA and export factor binding protein |
| 1083 | AAEL002853 | ccaat/enhancer binding protein |
| 1084 | AAEL003800 | hypothetical protein |
| 1085 | AAEL002551 | DNA topoisomerase type I |
| 1086 | AAEL008738 | DEAD box ATP-dependent RNA helicase |
| 1087 | AAEL000193 | histone-lysine n-methyltransferase |
| 1088 | AAEL001912 | forkhead protein/forkhead protein domain |
| 1089 | AAEL002359 | homeobox protein onecut |
| 1090 | AAEL006473 | arginine/serine-rich splicing factor |
| 1091 | AAEL007801 | exonuclease |
| 1092 | AAEL003985 | small nuclear ribonucleoprotein, core, putative |
| 1093 | AAEL010642 | poly (A)-binding protein, putative |
| 1094 | AAEL001280 | 28S ribosomal protein S15, mitochondrial precursor |
| 1095 | AAEL015236 | signal recognition particle, 9 kD-subunit, putative |
| 1096 | AAEL015045 | transcription factor IIIA, putative |
| 1097 | AAEL001363 | small nuclear ribonucleoprotein Sm D1, putative |
| 1098 | AAEL005888 | DNA polymerase theta |
| 1099 | AAEL007885 | translation initiation factor-3 (IF3), putative |
| 1100 | AAEL006582 | calcium-transporting ATPase sarcoplasmic/endoplasmic |
| | | reticulum type |
| 1101 | AAEL005392 | dihydropyridine-sensitive l-type calcium channel |
| 1102 | AAEL003393 | ATP synthase beta subunit |
| 1103 | AAEL008928 | inward-rectifying potassium channel |
| 1104 | AAEL010361 | rer1 protein |
| 1105 | AAEL005043 | ATP-dependent bile acid permease |
| 1106 | AAEL010470 | calcineurin b subunit |
| 1107 | AAEL004141 | phosphatidylinositol transfer protein/retinal degeneration b |
| | | protein |
| 1108 | AAEL011657 | importin alpha |
| 1109 | AAEL007971 | tyrosine transporter |
| 1110 | AAEL009088 | liquid facets |
| 1111 | AAEL000567 | Facilitated trehalose transporter Tret1 |
| 1112 | AAEL003789 | exportin, putative |
| 1113 | AAEL010608 | succinate dehydrogenase |
| 1114 | AAEL013704 | beta-arrestin 1, |
| 1115 | AAEL013614 | clathrin heavy chain |
| 1116 | AAEL002061 | cation-transporting ATPase 13a1 (g-box binding protein) |
| 1117 | AAEL000417 | monocarboxylate transporter |
| 1118 | AAEL004743 | multidrug resistance protein 2 (ATP-binding |
| | | cassette protein c) |
| 1119 | AAEL002412 | monocarboxylate transporter |
| 1120 | AAEL008587 | glutamate receptor, ionotropic, N-methyl d-aspartate |
| 1121 | AAEL010481 | sugar transporter |
| 1122 | AAEL006047 | histamine-gated chloride channel subunit |
| 1123 | AAEL010823 | ATP synthase delta chain |
| 1124 | AAEL004025 | glucose dehydrogenase |
| 1125 | AAEL003626 | sodium/chloride dependent amino acid transporter |
| 1126 | AAEL005859 | amino acid transporter |
| 1127 | AAEL000435 | THO complex, putative |
| 1128 | AAEL004620 | sorting nexin |
| 1129 | AAEL011423 | sugar transporter |
| 1130 | AAEL013215 | sulfonylurea receptor/ABC transporter |
| 1131 | AAEL001313 | conserved hypothetical protein |
| 1132 | AAEL003025 | hypothetical protein |
| 1133 | AAEL004447 | hypothetical protein |
| 1134 | AAEL004149 | hypothetical protein |
| 1135 | AAEL011064 | hypothetical protein |
| 1136 | AAEL002757 | hypothetical protein |
| 1137 | AAEL009776 | conserved hypothetical protein |
| 1138 | AAEL002835 | conserved hypothetical protein |
| 1139 | AAEL014693 | conserved hypothetical protein |
| 1140 | AAEL012203 | conserved hypothetical protein |
| 1141 | AAEL005867 | conserved hypothetical protein |
| 1142 | AAEL007539 | hypothetical protein |
| 1143 | AAEL001409 | conserved hypothetical protein |
| 1144 | AAEL002963 | conserved hypothetical protein |
| 1145 | AAEL010308 | hypothetical protein |
| 1146 | AAEL009386 | hypothetical protein |
| 1147 | AAEL011153 | hypothetical protein |
| 1148 | AAEL006863 | hypothetical protein |
| 1149 | AAEL001786 | hypothetical protein |
| 1150 | AAEL007606 | hypothetical protein |
| 1151 | AAEL007242 | conserved hypothetical protein |
| 1152 | AAEL008054 | conserved hypothetical protein |
| 1153 | AAEL014415 | conserved hypothetical protein |
| 1154 | AAEL011703 | conserved hypothetical protein |
| 1155 | AAEL002169 | conserved hypothetical protein |
| 1156 | AAEL002168 | conserved hypothetical protein |
| 1157 | AAEL010445 | hypothetical protein |
| 1158 | AAEL004583 | conserved hypothetical protein |
| 1159 | AAEL003373 | hypothetical protein |
| 1160 | AAEL005843 | conserved hypothetical protein |
| 1161 | AAEL012302 | conserved hypothetical protein |
| 1162 | AAEL012293 | conserved hypothetical protein |
| 1163 | AAEL007817 | hypothetical protein |
| 1164 | AAEL002327 | hypothetical protein |
| 1165 | AAEL010015 | hypothetical protein |
| 1166 | AAEL004800 | hypothetical protein |
| 1167 | AAEL013800 | conserved hypothetical protein |
| 1168 | AAEL007454 | conserved hypothetical protein |
| 1169 | AAEL001581 | conserved hypothetical protein |
| 1170 | AAEL001376 | hypothetical protein |
| 1171 | AAEL004854 | conserved hypothetical protein |
| 1172 | AAEL007015 | conserved hypothetical protein |
| 1173 | AAEL000258 | conserved hypothetical protein |
| 1174 | AAEL002543 | conserved hypothetical protein |
| 1175 | AAEL006520 | hypothetical protein |
| 1176 | AAEL006275 | conserved hypothetical protein |
| 1177 | AAEL014294 | conserved hypothetical protein |
| 1178 | AAEL014022 | conserved hypothetical protein |
| 1179 | AAEL004832 | conserved hypothetical protein |
| 1180 | AAEL000316 | hypothetical protein |
| 1181 | AAEL012754 | hypothetical protein |
| 1182 | AAEL005007 | hypothetical protein |
| 1183 | AAEL009163 | conserved hypothetical protein |
| 1184 | AAEL001495 | conserved hypothetical protein |
| 1185 | AAEL004934 | hypothetical protein |
| 1186 | AAEL007071 | conserved hypothetical protein |
| 1187 | AAEL004363 | conserved hypothetical protein |
| 1188 | AAEL007433 | conserved hypothetical protein |
| 1189 | AAEL010025 | conserved hypothetical protein |
| 1190 | AAEL002984 | hypothetical protein |
| 1191 | AAEL003126 | conserved hypothetical protein |
| 1192 | AAEL008154 | hypothetical protein |
| 1193 | AAEL000649 | conserved hypothetical protein |
| 1194 | AAEL013724 | conserved hypothetical protein |
| 1195 | AAEL012854 | hypothetical protein |
| 1196 | AAEL012858 | hypothetical protein |
| 1197 | AAEL014950 | spaetzle-like cytokine |
| 1198 | AAEL011066 | hypothetical protein |
| 1199 | AAEL009896 | hypothetical protein |
| 1200 | AAEL001727 | hypothetical protein |
| 1201 | AAEL001921 | hypothetical protein |
| 1202 | AAEL012396 | conserved hypothetical protein |
| 1203 | AAEL005233 | hypothetical protein |
| 1204 | AAEL015446 | conserved hypothetical protein |
| 1205 | AAEL007550 | conserved hypothetical protein |
| 1206 | AAEL011886 | hypothetical protein |
| 1207 | AAEL006761 | hypothetical protein |
| 1208 | AAEL003778 | conserved hypothetical protein |
| 1209 | AAEL002931 | hypothetical protein |
| 1210 | AAEL013303 | conserved hypothetical protein |
| 1211 | AAEL007414 | conserved hypothetical protein |
| 1212 | AAEL003693 | hypothetical protein |
| 1213 | AAEL010150 | conserved hypothetical protein |
| 1214 | AAEL004498 | hypothetical protein |
| 1215 | AAEL011598 | hypothetical protein |
| 1216 | AAEL003798 | hypothetical protein |
| 1217 | AAEL010746 | hypothetical protein |
| 1218 | AAEL011266 | hypothetical protein |
| 1219 | AAEL001271 | conserved hypothetical protein |
| 1220 | AAEL005193 | hypothetical protein |
| 1221 | AAEL007805 | hypothetical protein |
| 1222 | AAEL013304 | conserved hypothetical protein |
| 1223 | AAEL008142 | hypothetical protein |
| 1224 | AAEL009322 | hypothetical protein |
| 1225 | AAEL004018 | conserved hypothetical protein |
| 1226 | AAEL006606 | hypothetical protein |
| 1227 | AAEL007437 | conserved hypothetical protein |
| 1228 | AAEL013684 | conserved hypothetical protein |
| 1229 | AAEL007751 | predicted protein |
| 1230 | AAEL005623 | hypothetical protein |
| 1231 | AAEL006896 | hypothetical protein |
| 1232 | AAEL003190 | hypothetical protein |
| 1233 | AAEL007886 | hypothetical protein |
| 1234 | AAEL004943 | conserved hypothetical protein |
| 1235 | AAEL004561 | conserved hypothetical protein |
| 1236 | AAEL005264 | hypothetical protein |
| 1237 | AAEL011330 | conserved hypothetical protein |
| 1238 | AAEL000186 | conserved hypothetical protein |
| 1239 | AAEL012931 | conserved hypothetical protein |
| 1240 | AAEL000561 | hypothetical protein |
| 1241 | AAEL002921 | conserved hypothetical protein |
| 1242 | AAEL001162 | conserved hypothetical protein |
| 1243 | AAEL012361 | conserved hypothetical protein |
| 1244 | AAEL013426 | hypothetical protein |
| 1245 | AAEL013935 | conserved hypothetical protein |
| 1246 | AAEL003264 | conserved hypothetical protein |
| 1247 | AAEL005972 | hypothetical protein |
| 1248 | AAEL008680 | Ubiquitin-related modifier 1 homolog |
| 1249 | AAEL003088 | hypothetical protein |
| 1250 | AAEL009270 | hypothetical protein |
| 1251 | AAEL012878 | hypothetical protein |
| 1252 | AAEL013895 | conserved hypothetical protein |
| 1253 | AAEL003816 | hypothetical protein |
| 1254 | AAEL011636 | hypothetical protein |
| 1255 | AAEL004775 | conserved hypothetical protein |
| 1256 | AAEL006225 | conserved hypothetical protein |
| 1257 | AAEL009892 | conserved hypothetical protein |
| 1258 | AAEL011640 | hypothetical protein |
| 1259 | AAEL009767 | conserved hypothetical protein |
| 1260 | AAEL003113 | conserved hypothetical protein |
| 1261 | AAEL008557 | conserved hypothetical protein |
| 1262 | AAEL002856 | conserved hypothetical protein |
| 1263 | AAEL004250 | conserved hypothetical protein |
| 1264 | AAEL003451 | conserved hypothetical protein |
| 1265 | AAEL010249 | conserved hypothetical protein |
| 1266 | AAEL014937 | hypothetical protein |
| 1267 | AAEL004552 | conserved hypothetical protein |
| 1268 | AAEL005000 | conserved hypothetical protein |
| 1269 | AAEL010768 | conserved hypothetical protein |
| 1270 | AAEL004960 | hypothetical protein |
| 1271 | AAEL003822 | conserved hypothetical protein |
| 1272 | AAEL004473 | conserved hypothetical protein |
| 1273 | AAEL009952 | hypothetical protein |
| 1274 | AAEL002109 | conserved hypothetical protein |
| 1275 | AAEL007849 | conserved hypothetical protein |
| 1276 | AAEL010507 | hypothetical protein |
| 1277 | AAEL015340 | hypothetical protein |
| 1278 | AAEL013725 | conserved hypothetical protein |
| 1279 | AAEL000526 | conserved hypothetical protein |
| 1280 | AAEL010770 | hypothetical protein |
| 1281 | AAEL015507 | conserved hypothetical protein |
| 1282 | AAEL001573 | conserved hypothetical protein |
| 1283 | AAEL007045 | conserved hypothetical protein |
| 1284 | AAEL008403 | conserved hypothetical protein |
| 1285 | AAEL007859 | conserved hypothetical protein |
| 1286 | AAEL011635 | conserved hypothetical protein |
| 1287 | AAEL008059 | conserved hypothetical protein |
| 1288 | AAEL014633 | conserved hypothetical protein |
| 1289 | AAEL011119 | hypothetical protein |
| 1290 | AAEL005640 | conserved hypothetical protein |
| 1291 | AAEL013740 | hypothetical protein |
| 1292 | AAEL009440 | conserved hypothetical protein |
| 1293 | AAEL002087 | conserved hypothetical protein |
| 1294 | AAEL008436 | conserved hypothetical protein |
| 1325 | AAEL007698 | |
| | (AuB) | |
| 1326 | AAEL003832 | |
| 1327 | AAEL007562 | |
| 1328 | AAEL010179 | |
| 1329 | AAEL000598 | |

Homologs and orthologs of gene symbols GNBPA2, GNBPB4, GNBPB6, CLIPB13B, SPZ5, PGRPLD, SOCS, SOCS16D, SOCS44A, SUMO, CECG, GAM, LYSC, DOME, HOP, STAT, REL1A and CTLMA12 are also contemplated in accordance with the present teachings.

In some embodiments, the pathogen resistance gene products include, but are not limited to sequences of AAEL000652, AAEL009178, AAEL003253, AAEL006936, AAEL000393, AAEL006794, AAEL011455, AAEL015312 or AAEL001702 or their corresponding homologs and orthologs.

According to one embodiment, the pathogen resistance gene product that is downregulated is as set forth in SEQ ID NO: 3, 5, 31, 98, 102, 122, 271, 291 or 299.

According to one embodiment, the pathogen resistance gene is selected from the group consisting of AAEL007768 (MyD88), AAEL000709 (Cactus), AAEL007698 (AuB), AAEL003832, AAEL007562, Rel1A (AAEL007696), AAEL000598 and AAEL010179.

According to one embodiment, pathogen resistance gene is selected from the group consisting of SEQ ID NOs: 964, 945, 1325, 1326, 1327, 1328 and 1329.

It will be appreciated that more than one gene may be targeted in order to maximize the cytotoxic effect on the mosquitoes.

As used herein, the term “downregulates an expression” or “downregulating expression” refers to causing, directly or indirectly, reduction in the transcription of a desired gene, reduction in the amount, stability or translatability of transcription products (e.g. RNA) of the gene, and/or reduction in translation of the polypeptide(s) encoded by the desired gene.

Downregulating expression of a pathogen resistance gene product of a mosquito can be monitored, for example, by direct detection of gene transcripts (for example, by PCR), by detection of polypeptide(s) encoded by the gene (for example, by Western blot or immunoprecipitation), by detection of biological activity of polypeptides encode by the gene (for example, catalytic activity, ligand binding, and the like), or by monitoring changes in the mosquitoes (for example, reduced motility of the mosquito etc). Additionally or alternatively downregulating expression of a pathogen resistance gene product may be monitored by measuring pathogen levels (e.g. viral levels, bacterial levels etc.) in the mosquitoes as compared to wild type (i.e. control) mosquitoes not treated by the agents of the invention.

Thus, according to some aspects of the invention there is provided an isolated nucleic acid agent comprising a nucleic acid sequence which specifically downregulates the expression of at least one mosquito pathogen resistance gene product.

According to one embodiment, the agent is a polynucleotide agent, such as an RNA silencing agent.

As used herein, the term “RNA silencing agent” refers to an RNA which is capable of inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

In some embodiments of the invention, the nucleic acid agent is a double stranded RNA (dsRNA). As used herein the term “dsRNA” relates to two strands of anti-parallel polyribonucleic acids held together by base pairing. The two strands can be of identical length or of different lengths provided there is enough sequence homology between the two strands that a double stranded structure is formed with at least 80%, 90%, 95% or 100% complementarity over the entire length. According to an embodiment of the invention, there are no overhangs for the dsRNA molecule. According to another embodiment of the invention, the dsRNA molecule comprises overhangs. According to other embodiments, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed.

It will be noted that the dsRNA can be defined in terms of the nucleic acid sequence of the DNA encoding the target gene transcript, and it is understood that a dsRNA sequence corresponding to the coding sequence of a gene comprises an RNA complement of the gene's coding sequence, or other sequence of the gene which is transcribed into RNA.

The inhibitory RNA sequence can be greater than 90% identical, or even 100% identical, to the portion of the target gene transcript. Alternatively, the duplex region of the RNA may be defined functionally as a nucleotide sequence that is capable of hybridizing with a portion of the target gene transcript under stringent conditions (e.g., 400 mM NaCl, 40 mM PIPES pH 6.4, 1 mM EDTA, 60 degrees C. hybridization for 12-16 hours; followed by washing). The length of the double-stranded nucleotide sequences complementary to the target gene transcript may be at least about 18, 19, 21, 25, 50, 100, 200, 300, 400, 491, 500, 550, 600, 650, 700, 750, 800, 900, 1000 or more bases. In some embodiments of the invention, the length of the double-stranded nucleotide sequence is approximately from about 18 to about 1000, about 18 to about 750, about 18 to about 510, about 18 to about 400, about 18 to about 250 nucleotides in length.

The term “corresponds to” as used herein means a polynucleotide sequence homologous to all or a portion of a reference polynucleotide sequence. In contradistinction, the term “complementary to” is used herein to mean that the complementary sequence is homologous to all or a portion of a reference polynucleotide sequence. For example, the nucleotide sequence “TATAC” corresponds to a reference sequence “TATAC” and is complementary to a reference sequence “GTATA”.

The present teachings relate to various lengths of dsRNA, whereby the shorter version i.e., x is shorter or equals 50 bp (e.g., 17-50), is referred to as siRNA or miRNA. Longer dsRNA molecules of 51-600 are referred to herein as dsRNA, which can be further processed for siRNA molecules. According to some embodiments, the nucleic acid sequence of the dsRNA is greater than 15 base pairs in length. According to yet other embodiments, the nucleic acid sequence of the dsRNA is 19-25 base pairs in length, 30-100 base pairs in length, 100-250 base pairs in length or 100-500 base pairs in length. According to still other embodiments, the dsRNA is 500-800 base pairs in length, 700-800 base pairs in length, 300-600 base pairs in length, 350-500 base pairs in length or 400-450 base pairs in length. In some embodiments, the dsRNA is 400 base pairs in length. In some embodiments, the dsRNA is 750 base pairs in length.

The term “siRNA” refers to small inhibitory RNA duplexes (generally between 17-30 basepairs, but also longer e.g., 31-50 bp) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of a siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., a siRNA) may be connected to form a hairpin or stem-loop structure (e.g., a shRNA). Thus, as mentioned the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-UUCAAGAGA-3′ (Brummelkamp, T. R. et al. (2002) Science 296: 550, SEQ ID NO: 302) and 5′-UUUGUGUAG-3′ (Castanotto, D. et al. (2002) RNA 8:1454, SEQ ID NO: 303). It will be recognized by one of skill in the art that the resulting single chain oligonucleotide forms a stem-loop or hairpin structure comprising a double-stranded region capable of interacting with the RNAi machinery.

As used herein, the phrase “microRNA (also referred to herein interchangeably as “miRNA” or “miR”) or a precursor thereof” refers to a microRNA (miRNA) molecule acting as a post-transcriptional regulator. Typically, the miRNA molecules are RNA molecules of about 20 to 22 nucleotides in length which can be loaded into a RISC complex and which direct the cleavage of another RNA molecule, wherein the other RNA molecule comprises a nucleotide sequence essentially complementary to the nucleotide sequence of the miRNA molecule.

Typically, a miRNA molecule is processed from a “pre-miRNA” or as used herein a precursor of a pre-miRNA molecule by proteins, such as DCL proteins, and loaded onto a RISC complex where it can guide the cleavage of the target RNA molecules.

Pre-microRNA molecules are typically processed from pri-microRNA molecules (primary transcripts). The single stranded RNA segments flanking the pre-microRNA are important for processing of the pri-miRNA into the pre-miRNA. The cleavage site appears to be determined by the distance from the stem-ssRNA junction (Han et al. 2006, Cell 125, 887-901, 887-901).

As used herein, a “pre-miRNA” molecule is an RNA molecule of about 100 to about 200 nucleotides, preferably about 100 to about 130 nucleotides which can adopt a secondary structure comprising an imperfect double stranded RNA stem and a single stranded RNA loop (also referred to as “hairpin”) and further comprising the nucleotide sequence of the miRNA (and its complement sequence) in the double stranded RNA stem. According to a specific embodiment, the miRNA and its complement are located about 10 to about 20 nucleotides from the free ends of the miRNA double stranded RNA stem. The length and sequence of the single stranded loop region are not critical and may vary considerably, e.g. between 30 and 50 nucleotides in length. The complementarity between the miRNA and its complement need not be perfect and about 1 to 3 bulges of unpaired nucleotides can be tolerated. The secondary structure adopted by an RNA molecule can be predicted by computer algorithms conventional in the art such as mFOLD. The particular strand of the double stranded RNA stem from the pre-miRNA which is released by DCL activity and loaded onto the RISC complex is determined by the degree of complementarity at the 5′ end, whereby the strand which at its 5′ end is the least involved in hydrogen bounding between the nucleotides of the different strands of the cleaved dsRNA stem is loaded onto the RISC complex and will determine the sequence specificity of the target RNA molecule degradation. However, if empirically the miRNA molecule from a particular synthetic pre-miRNA molecule is not functional (because the “wrong” strand is loaded on the RISC complex), it will be immediately evident that this problem can be solved by exchanging the position of the miRNA molecule and its complement on the respective strands of the dsRNA stem of the pre-miRNA molecule. As is known in the art, binding between A and U involving two hydrogen bounds, or G and U involving two hydrogen bounds is less strong that between G and C involving three hydrogen bounds.

Naturally occurring miRNA molecules may be comprised within their naturally occurring pre-miRNA molecules but they can also be introduced into existing pre-miRNA molecule scaffolds by exchanging the nucleotide sequence of the miRNA molecule normally processed from such existing pre-miRNA molecule for the nucleotide sequence of another miRNA of interest. The scaffold of the pre-miRNA can also be completely synthetic. Likewise, synthetic miRNA molecules may be comprised within, and processed from, existing pre-miRNA molecule scaffolds or synthetic pre-miRNA scaffolds. Some pre-miRNA scaffolds may be preferred over others for their efficiency to be correctly processed into the designed microRNAs, particularly when expressed as a chimeric gene wherein other DNA regions, such as untranslated leader sequences or transcription termination and polyadenylation regions are incorporated in the primary transcript in addition to the pre-microRNA.

According to the present teachings, the dsRNA molecules may be naturally occurring or synthetic.

The dsRNA can be a mixture of long and short dsRNA molecules such as, dsRNA, siRNA, siRNA+dsRNA, siRNA+miRNA, or a combination of same.

The nucleic acid agent is designed for specifically targeting a target gene of interest (e.g. a mosquito pathogen resistance gene). It will be appreciated that the nucleic acid agent can be used to downregulate one or more target genes (e.g. as described in detail above). If a number of target genes are targeted, a heterogenic composition which comprises a plurality of nucleic acid agents for targeting a number of target genes is used. Alternatively the plurality of nucleic acid agents is separately formulated. According to a specific embodiment, a number of distinct nucleic acid agent molecules for a single target are used, which may be used separately or simultaneously (i.e., co-formulation) applied.

For example, in order to silence the expression of an mRNA of interest, synthesis of the dsRNA suitable for use with some embodiments of the invention can be selected as follows. First, the mRNA sequence is scanned including the 3′ UTR and the 5′ UTR. Second, the mRNA sequence is compared to an appropriate genomic database using any sequence alignment software, such as the BLAST software available from the NCBI server (wwwdotncbidotnlmdotnihdotgov/BLAST/). Putative regions in the mRNA sequence which exhibit significant homology to other coding sequences are filtered out.

Qualifying target sequences are selected as template for dsRNA synthesis. Preferred sequences are those that have as little homology to other genes in the genome to reduce an “off-target” effect.

It will be appreciated that the RNA silencing agent of some embodiments of the invention need not be limited to those molecules containing only RNA, but further encompasses chemically-modified nucleotides and non-nucleotides.

According to one embodiment, the dsRNA specifically targets a gene selected from the group consisting of AAEL007768 (MyD88), AAEL000709 (Cactus), AAEL007698 (AuB), AAEL003832, AAEL007562, Rel1A (AAEL007696), AAEL000598 and AAEL010179.

According to one embodiment, the dsRNA is selected from the group consisting of SEQ ID NOs: 1315-1324 and 1330.

The dsRNA may be synthesized using any method known in the art, including either enzymatic syntheses or solid-phase syntheses. These are especially useful in the case of short polynucleotide sequences with or without modifications as explained above. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Md.; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.

According to a specific embodiment, the nucleic acid agent is provided to the mosquito in a configuration devoid of a heterologous promoter for driving recombinant expression of the dsRNA (exogenous), rendering the nucleic acid molecule of the instant invention a naked molecule. The nucleic acid agent may still comprise modifications that may affect its stability and bioavailability (e.g., PNA).

The term “recombinant expression” refers to an expression from a nucleic acid construct.

As used herein “devoid of a heterologous promoter for driving expression of the dsRNA” means that the molecule doesn't include a cis-acting regulatory sequence (e.g., heterologous) transcribing the dsRNA. As used herein the term “heterologous” refers to exogenous, not-naturally occurring within a native cell of the mosquito or in a cell in which the dsRNA is fed to the larvae or mosquito (such as by position of integration, or being non-naturally found within the cell).

The nucleic acid agent can be further comprised within a nucleic acid construct comprising additional regulatory elements. Thus, according to some embodiments of aspects of the invention there is provided a nucleic acid construct comprising isolated nucleic acid agent comprising a nucleic acid sequence which specifically reduces the expression of at least one mosquito pathogen resistance gene product.

Although the instant teachings mainly concentrate on the use of dsRNA which is not comprised in or transcribed from an expression vector (naked), the present teachings also contemplate an embodiment wherein the nucleic acid agent is ligated into a nucleic acid construct comprising additional regulatory elements. Thus, according to some embodiments of the invention there is provided a nucleic acid construct comprising an isolated nucleic acid agent comprising a nucleic acid sequence.

For transcription from an expression cassette, a regulatory region (e.g., promoter, enhancer, silencer, leader, intron and polyadenylation) may be used to modulate the transcription of the RNA strand (or strands). Therefore, in one embodiment, there is provided a nucleic acid construct comprising the nucleic acid agent. The nucleic acid construct can have polynucleotide sequences constructed to facilitate transcription of the RNA molecules of the present invention operably linked to one or more promoter sequences functional in a mosquito cell. The polynucleotide sequences may be placed under the control of an endogenous promoter normally present in the mosquito genome. The polynucleotide sequences of the present invention, under the control of an operably linked promoter sequence, may further be flanked by additional sequences that advantageously affect its transcription and/or the stability of a resulting transcript. Such sequences are generally located upstream of the promoter and/or downstream of the 3′ end of the expression construct. The term “operably linked”, as used in reference to a regulatory sequence and a structural nucleotide sequence, means that the regulatory sequence causes regulated expression of the linked structural nucleotide sequence. “Regulatory sequences” or “control elements” refer to nucleotide sequences located upstream, within, or downstream of a structural nucleotide sequence, and which influence the timing and level or amount of transcription, RNA processing or stability, or translation of the associated structural nucleotide sequence. Regulatory sequences may include promoters, translation leader sequences, introns, enhancers, stem-loop structures, repressor binding sequences, termination sequences, pausing sequences, polyadenylation recognition sequences, and the like.

It will be appreciated that the nucleic acid agents can be delivered to the mosquito larva in a variety of ways.

According to one embodiment, the composition of some embodiments comprises cells, which comprise the nucleic acid agent.

As used herein the term “cell” or “cells” refers to a mosquito larva ingestible cell.

Examples of such cells include, but are not limited to, cells of phytoplankton (e.g., algae), fungi (e.g., Legendium giganteum), bacteria, and zooplankton such as rotifers.

Specific examples include, bacteria (e.g., cocci and rods), filamentous algae and detritus.

The choice of the cell may depend on the target larvae.

Analyzing the gut content of mosquitoes and larvae may be used to elucidate their preferred diet. The skilled artisan knows how to characterize the gut content. Typically the gut content is stained such as by using a fluorochromatic stain, 4′,6-diamidino-2-phenylindole or DAPI.

Cells of particular interest are the prokaryotes and the lower eukaryotes, such as fungi. Illustrative prokaryotes, both Gram-negative and Gram-positive, include Enterobacteriaceae; Bacillaceae; Rhizobiceae; Spirillaceae; Lactobacillaceae; and phylloplane organisms such as members of the Pseudomonadaceae.

An exemplary list includes Bacillus spp., including B. megaterium, B. subtilis; B. cereus, Bacillus thuringiensis, Escherichia spp., including E. coli, and/or Pseudomonas spp., including P. cepacia, P. aeruginosa, and P. fluorescens.

Among eukaryotes are fungi, such as Phycomycetes and Ascomycetes, which includes yeast, such as Schizosaccharomyces; and Basidiomycetes, Rhodotorula, Aureobasidium, Sporobolomyces, Saccharomyces spp., and Sporobolomyces spp.

According to a specific embodiment, the cell is an algal cell.

Various algal species can be used in accordance with the teachings of the invention since they are a significant part of the diet for many kinds of mosquito larvae that feed opportunistically on microorganisms as well as on small aquatic animals such as rotifers.

Examples of algae that can be used in accordance with the present teachings include, but are not limited to, blue-green algae as well as green algae.

According to a specific embodiment, the algal cell is a cyanobacterium cell which is in itself toxic to mosquitoes as taught by Marten 2007 Biorational Control of Mosquitoes. American mosquito control association Bulletin No. 7.

Specific examples of algal cells which can be used in accordance with the present teachings are provided in Marten, G. G. (1986) Mosquito control by plankton management: the potential of indigestible green algae. Journal of Tropical Medicine and Hygiene, 89: 213-222, and further listed infra.

### Green Algae

Actinastrum hantzschii, Ankistrodesmus falcatus, Ankistrodesmus spiralis, Aphanochaete elegans, Chlamydomonas sp., Chlorella ellipsoidea, Chlorella pyrenoidosa, Chlorella variegate, Chlorococcum hypnosporum, Chodatella brevispina, Closterium acerosum, Closteriopsis acicularis, Coccochloris peniocystis, Crucigenia lauterbornii, Crucigenia tetrapedia, Coronastrum ellipsoideum, Cosmarium botrytis, Desmidium swartzii, Eudorina elegans, Gloeocystis gigas, Golenkinia minutissima, Gonium multicoccum, Nannochloris oculata, Oocystis mars sonii, Oocystis minuta, Oocystis pusilla, Palmella texensis, Pandorina morum, Paulschulzia pseudovolvox, Pediastrum clathratum, Pediastrum duplex, Pediastrum simplex, Planktosphaeria gelatinosa, Polyedriopsis spinulosa, Pseudococcomyxa adhaerans, Quadrigula closterioides, Radiococcus nimbatus, Scenedesmus basiliensis, Spirogyra pratensis, Staurastrum gladiosum, Tetraedron bitridens, Trochiscia hystrix.

### Blue-Green Algae

Anabaena catenula, Anabaena spiroides, Chroococcus turgidus, Cylindrospermum licheniforme, Bucapsis sp. (U. Texas No. 1519), Lyngbya spiralis, Microcystis aeruginosa, Nodularia spumigena, Nostoc linckia, Oscillatoria lutea, Phormidiumfaveolarum, Spinilina platensis.

### Other

Compsopogon coeruleus, CTyptomonas ovata, Navicula pelliculosa.

The nucleic acid agent is introduced into the cells. To this end cells are typically selected exhibiting natural competence or are rendered competent, also referred to as artificial competence.

Competence is the ability of a cell to take up nucleic acid molecules e.g., the nucleic acid agent, from its environment.

A number of methods are known in the art to induce artificial competence.

Thus, artificial competence can be induced in laboratory procedures that involve making the cell passively permeable to the nucleic acid agent by exposing it to conditions that do not normally occur in nature. Typically the cells are incubated in a solution containing divalent cations (e.g., calcium chloride) under cold conditions, before being exposed to a heat pulse (heat shock).

Electroporation is another method of promoting competence. In this method the cells are briefly shocked with an electric field (e.g., 10-20 kV/cm) which is thought to create holes in the cell membrane through which the nucleic acid agent may enter. After the electric shock the holes are rapidly closed by the cell's membrane-repair mechanisms.

Yet alternatively or additionally, cells may be treated with enzymes to degrade their cell walls, yielding. These cells are very fragile but take up foreign nucleic acids at a high rate.

Exposing intact cells to alkali cations such as those of cesium or lithium allows the cells to take up nucleic acids. Improved protocols use this transformation method, while employing lithium acetate, polyethylene glycol, and single-stranded nucleic acids. In these protocols, the single-stranded molecule preferentially binds to the cell wall in yeast cells, preventing double stranded molecule from doing so and leaving it available for transformation.

Enzymatic digestion or agitation with glass beads may also be used to transform cells.

Particle bombardment, microprojectile bombardment, or biolistics is yet another method for artificial competence. Particles of gold or tungsten are coated with the nucleic acid agent and then shot into cells.

Astier C R Acad Sci Hebd Seances Acad Sci D. 1976 Feb. 23; 282(8):795-7, which is hereby incorporated by reference in its entirety, teaches transformation of a unicellular, facultative chemoheterotroph blue-green Algae, Aphanocapsa 6714. The recipient strain becomes competent when the growth reaches its second, slower, exponential phase.

Vázquez-Acevedo M¹Mitochondrion. 2014 Feb. 21. pii: 51567-7249(14)00019-1. doi: 10.1016/j.mito.2014.02.005, which is hereby incorporated by reference in its entirety, teaches transformation of algal cells e.g., Chlamydomonas reinhardtii, Polytomella sp. and Volvox carteri by generating import-competent mitochondria.

According to a specific embodiment the composition of the invention comprises an RNA binding protein.

According to a specific embodiment, the dsRNA binding protein (DRBP) comprises any of the family of eukaryotic, prokaryotic, and viral-encoded products that share a common evolutionarily conserved motif specifically facilitating interaction with dsRNA. Polypeptides which comprise dsRNA binding domains (DRBDs) may interact with at least 11 bp of dsRNA, an event that is independent of nucleotide sequence arrangement. More than 20 DRBPs have been identified and reportedly function in a diverse range of critically important roles in the cell. Examples include the dsRNA-dependent protein kinase PKR that functions in dsRNA signaling and host defense against virus infection and DICER.

Alternatively or additionally, an siRNA binding protein may be used as taught in U.S. Pat. Application No. 20140045914, which is herein incorporated by reference in its entirety.

According to a specific embodiment the RNA binding protein is the p19 RNA binding protein. The protein may increase in vivo stability of an siRNA molecule by coupling it at a binding site where the homodimer of the p19 RNA binding proteins is formed and thus protecting the siRNA from external attacks and accordingly, it can be utilized as an effective siRNA delivery vehicle.

According to a specific embodiment, the RNA binding protein may be attached to a target-oriented peptide.

According to a specific embodiment, the target-oriented peptide is located on the surface of the siRNA binding protein.

According to specific embodiments of the invention, whole cell preparations, cell extracts, cell suspensions, cell homogenates, cell lysates, cell supernatants, cell filtrates, or cell pellets of cell cultures of cells comprising the nucleic acid agent can be used.

The composition of some embodiments of the invention may further comprise at least one of a surface-active agent, an inert carrier vehicle, a preservative, a humectant, a feeding stimulant, an attractant, an encapsulating agent, a binder, an emulsifier, a dye, an ultra-violet protector, a buffer, a flow agent or fertilizer, micronutrient donors.

According to a specific embodiment, the cells are formulated by any means known in the art. The methods for preparing such formulations include, e.g., desiccation, lyophilization, homogenization, extraction, filtration, encapsulation centrifugation, sedimentation, or concentration of one or more cell types.

Additionally, the composition may be supplemented with larval food (food bait) or with excrements of farm animals, on which the mosquito larvae feed.

In one embodiment, the composition comprises an oil flowable suspension. For example, in some embodiments, oil flowable or aqueous solutions may be formulated to contain lysed or unlysed cells, spores, or crystals.

In a further embodiment, the composition may be formulated as a water dispersible granule or powder.

In yet a further embodiment, the compositions of the present invention may also comprise a wettable powder, spray, emulsion, colloid, aqueous or organic solution, dust, pellet, or colloidal concentrate. Dry forms of the compositions may be formulated to dissolve immediately upon wetting, or alternatively, dissolve in a controlled-release, sustained-release, or other time-dependent manner.

Alternatively or additionally, the composition may comprise an aqueous solution. Such aqueous solutions or suspensions may be provided as a concentrated stock solution which is diluted prior to application, or alternatively, as a diluted solution ready-to-apply. Such compositions may be formulated in a variety of ways. They may be employed as wettable powders, granules or dusts, by mixing with various inert materials, such as inorganic minerals (silicone or silicon derivatives, phyllosilicates, carbonates, sulfates, phosphates, and the like) or botanical materials (powdered corncobs, rice hulls, walnut shells, and the like).

The formulations may include spreader-sticker adjuvants, stabilizing agents, other pesticidal additives, or surfactants. Liquid formulations may be employed as foams, suspensions, emulsifiable concentrates, or the like. The ingredients may include Theological agents, surfactants, emulsifiers, dispersants, or polymers.

As mentioned, the dsRNA of the invention may be administered as a naked dsRNA. Alternatively, the dsRNA of the invention may be conjugated to a carrier known to one of skill in the art, such as a transfection agent e.g. PEI or chitosan or a protein/lipid carrier.

The compositions may be formulated prior to administration in an appropriate means such as lyophilized, freeze-dried, microencapsulated, desiccated, or in an aqueous carrier, medium or suitable diluent, such as saline or other buffer. Suitable agricultural carriers can be solid, semi-solid or liquid and are well known in the art. The term “agriculturally-acceptable carrier” covers all adjuvants, e.g., inert components, dispersants, surfactants, tackifiers, binders, etc. that are ordinarily used in pesticide formulation technology.

According to one embodiment, the composition is formulated as a semi-solid such as in agarose (e.g. agarose cubes).

As mentioned, the nucleic acid agents can be delivered to the mosquito larva in various ways. Thus, administration of the composition to the mosquito larva may be carried out using any suitable or desired manual or mechanical technique for application of a composition comprising a nucleic acid agent, including but not limited to spraying, soaking, brushing, dressing, dripping, dipping, coating, spreading, applying as small droplets, a mist or an aerosol.

According to one embodiment, the composition is administered to the larvae by soaking or by spraying.

Soaking the larva with the composition can be effected for about 2 hours to 96 hours, about 2 hours to 84 hours, about 2 hours to 72 hours, for about 2 hours to 60 hours, about 2 hours to 48 hours, about 2 hours to 36 hours, about 2 hours to 24 hours, about 2 hours to 12 hours, 12 hours to 96 hours, about 12 hours to 84 hours, about 12 hours to 72 hours, for about 12 hours to 60 hours, about 12 hours to 48 hours, about 12 hours to 36 hours, about 12 hours to 24 hours, or about 24 hours to 48 hours.

According to a specific embodiment, the composition is administered to the larvae by soaking for 12-24 hours.

According to one embodiment, the composition is administered to the larvae by feeding.

Feeding the larva with the composition can be effected for about 2 hours to 120 hours, about 2 hours to 108 hours, about 2 hours to 96 hours, about 2 hours to 84 hours, about 2 hours to 72 hours, for about 2 hours to 60 hours, about 2 hours to 48 hours, about 2 hours to 36 hours, about 2 hours to 24 hours, about 2 hours to 12 hours, 12 hours to 24 hours, about 24 hours to 36 hours, about 24 hours to 48 hours, about 36 hours to 48 hours, for about 48 hours to 60 hours, about 60 hours to 72 hours, about 72 hours to 84 hours, about 84 hours to 96 hours, about 96 hours to 108 hours, or about 108 hours to 120 hours.

According to a specific embodiment, the composition is administered to the larvae by feeding for 48-96 hours.

According to one embodiment, feeding the larva with the composition is affected until the larva reaches pupa stage.

According to one embodiment, dsRNA is administered to the larva by soaking followed by feeding with food-containing dsRNA. Thus, for example, larvae (e.g. first, second, third or four instar larva, e.g. third instar larvae) are first treated (in groups of about 100 larvae) with dsRNA at a dose of about 0.001-5 μg/μL (e.g. 0.2 μg/μL), in a final volume of about 3 mL of dsRNA solution in autoclaved water. After soaking in the dsRNA solutions for about 12-48 hours (e.g. for 24 hrs) at 25-29° C. (e.g. 27° C.), the larvae are transferred into containers so as not to exceed concentration of about 200-500 larvae/1500 mL (e.g. 300 larvae/1500 mL) of chlorine-free tap water, and provided with food containing dsRNA (e.g. agarose cubes containing 300 μg of dsRNA, e.g. 1 μg of dsRNA/larvae). The larva are fed once a day until they reach pupa stage (e.g. for 2-5 days, e.g. four days). Larvae are also fed with additional food requirements, e.g. 2-10 mg/100 mL (e.g. 6 mg/100 mL) lab dog/cat diet suspended in water.

Feeding the larva can be effected using any method known in the art. Thus, for example, the larva may be fed with agrose cubes, chitosan nanoparticles, oral delivery or diet containing dsRNA.

Chitosan nanoparticles: A group of 15-20 3rd-instar mosquito larvae are transferred into a container (e.g. 500 ml glass beaker) containing 50-1000 ml, e.g. 100 ml, of deionized water. One sixth of the gel slices that are prepared from dsRNA (e.g. 32 μg of dsRNA) are added into each beaker. Approximately an equal amount of the gel slices are used to feed the larvae once a day for a total of 2-5 days, e.g. four days (see Insect Mol Biol. 2010 19(5):683-93).

Oral delivery of dsRNA: First instar larvae (less than 24 hrs old) are treated in groups of 10-100, e.g. 50, in a final volume of 25-100 μl of dsRNA, e.g. 75 μl of dsRNA, at various concentrations (ranging from 0.01 to 5 μg/μl, e.g. 0.02 to 0.5 μg/μ1-dsRNAs) in tubes e.g. 2 mL microfuge tube (see J Insect Sci. 2013; 13:69).

Diet containing dsRNA: larvae are fed a single concentration of 1-2000 ng dsRNA/mL, e.g. 1000 ng dsRNA/mL, diet in a diet overlay bioassay for a period of 1-10 days, e.g. 5 days (see PLoS One. 2012; 7(10): e47534.).

Diet containing dsRNA: Newly emerged larvae are starved for 1-12 hours, e.g. 2 hours, and are then fed with a single drop of 0.5-10 μl, e.g. 1 μl, containing 1-20 μg, e.g. 4 μg, dsRNA (1-20 μg of dsRNA/larva, e.g. 4 μg of dsRNA/larva) (see Appl Environ Microbiol. 2013 August; 79(15):4543-50).

Thus, according to a specific embodiment, the composition may be applied to standing water. The mosquito larva may be soaked in the water for several hours (1, 2, 3, 4, 5, 6 hours or more) to several days (1, 2, 3, 4 days or more) with or without the use of transfection reagents or dsRNA carriers.

Alternatively, the mosquito larva may be sprayed with an effective amount of the composition (e.g. via an aqueous solution).

If needed, the composition may be dissolved, suspended and/or diluted in a suitable solution (as described in detail above) before use.

The nucleic acid compositions of the invention may be employed in the method of the invention singly or in combination with other compounds, including, but not limited to, inert carriers that may be natural, synthetic, organic or inorganic, humectants, feeding stimulants, attractants, encapsulating agents (for example Algae, bacteria and yeast, nanoparticles), dsRNA binding proteins, binders, emulsifiers, dyes, sugars, sugar alcohols, starches, modified starches, dispersants, or combinations thereof may also be utilized in conjunction with the composition of some embodiments of the invention.

Compositions of the invention can be used to control (e.g. exterminate) mosquitoes. Such an application comprises administering to larvae of the mosquitoes an effective amount of the composition which renders an adult stage of the mosquitoes lethally susceptible to a pathogen, thereby controlling (e.g. exterminating) the mosquitoes.

Thus, regardless of the method of application, the amount of the active component(s) are applied at a effective amount for an adult stage of the mosquito to be lethally susceptible to a pathogen, which will vary depending on factors such as, for example, the specific mosquito to be controlled, the type of pathogen (bacteria, virus, protozoa, etc.), the water source to be treated, the environmental conditions, and the method, rate, and quantity of application of the composition.

The concentration of the composition that is used for environmental, systemic, or foliar application will vary widely depending upon the nature of the particular formulation, means of application, environmental conditions, and degree of biocidal activity.

Exemplary concentrations of dsRNA in the composition (e.g. for soaking) include, but are not limited to, about 1 pg-10 μg of dsRNA/μl, about 1 pg-1 μg of dsRNA/μl, about 1 pg-0.1 μg of dsRNA/μl, about 1 pg-0.01 μg of dsRNA/μl, about 1 pg-0.001 μg of dsRNA/μl, about 0.001 μg-10 μg of dsRNA/μl, about 0.001 μg-5 μg of dsRNA/μl, about 0.001 μg-1 μg of dsRNA/μl, about 0.001 μg-0.1 μg of dsRNA/μl, about 0.001 μg-0.01 μg of dsRNA/μl, about 0.01 μg-10 μg of dsRNA/μl, about 0.01 μg-5 μg of dsRNA/μl, about 0.01 μg-1 μg of dsRNA/μl, about 0.01 μg-0.1 μg of dsRNA/μl, about 0.1 μg-10 μg of dsRNA/μl, about 0.1 μg-5 μg of dsRNA/μl, about 0.5 μg-5 μg of dsRNA/μl, about 0.5 μg-10 μg of dsRNA/μl, about 1 iμg-5 iμg of dsRNA/μl, or about 1 μg-10 μg of dsRNA/μl.

When formulated as a feed, the dsRNA may be effected at a dose of 1 pg/larvae-1000 μg/larvae, 1 pg/larvae-500 μg/larvae, 1 pg/larvae-100 μg/larvae, 1 pg/larvae-10 μg/larvae, 1 pg/larvae-1 μg/larvae, 1 pg/larvae-0.1 μg/larvae, 1 pg/larvae-0.01 μg/larvae, 1 pg/larvae-0.001 μg/larvae, 0.001-1000 μg/larvae, 0.001-500 μg/larvae, 0.001-100 μg/larvae, 0.001-50 μg/larvae, 0.001-10 μg/larvae, 0.001-1 μg/larvae, 0.001-0.1 μg/larvae, 0.001-0.01 μg/larvae, 0.01-1000 μg/larvae, 0.01-500 μg/larvae, 0.01-100 μg/larvae, 0.01-50 μg/larvae, 0.01-10 μg/larvae, 0.01-1 μg/larvae, 0.01-0.1 μg/larvae, 0.1-1000 μg/larvae, 0.1-500 μg/larvae, 0.1-100 μg/larvae, 0.1-50 μg/larvae, 0.1-10 μg/larvae, 0.1-1 μg/larvae, 1-1000 μg/larvae, 1-500 μg/larvae, 1-100 μg/larvae, 1-50 μg/larvae, 1-10 μg/larvae, 10-1000 μg/larvae, 10-500 μg/larvae, 10-100 μg/larvae, 10-50 μg/larvae, 50-1000 μg/larvae, 50-500 μg/larvae, 50-400 μg/larvae, 50-300 μg/larvae, 100-500 μg/larvae, 100-300 μg/larvae, 200-500 μg/larvae, 200-300 μg/larvae, or 300-500 μg/larvae.

The mosquito larva food containing dsRNA may be prepared by any method known to one of skill in the art. Thus, for example, cubes of dsRNA-containing mosquito food may be prepared by first mixing 10-500 μg, e.g. 300 μg of dsRNA with 3 to 300 μg, e.g. 10 μg of a transfection agent e.g. Polyethylenimine 25 kDa linear (Polysciences) in 10-500 μL, e.g. 200 μL of sterile water. Alternatively, 2 different dsRNA (10-500 μg, e.g. 150 μg of each) plus 3 to 300 μg, e.g. 30 μg of Polyethylenimine may be mixed in 10-500 μL, e.g. 200 μL of sterile water. Alternatively, cubes of dsRNA-containing mosquito food may be prepared without the addition of transfection reagents. Then, a suspension of ground mosquito larval food (1-20 grams/100 mL e.g. 6 grams/100 mL) may be prepared with 2% agarose (Fisher Scientific). The food/agarose mixture can then be heated to 53-57° C., e.g. 55° C., and 10-500 μL, e.g. 200 μL of the mixture can then be transferred to the tubes containing 10-500 μL, e.g. 200 μL of dsRNA+PEI or dsRNA only. The mixture is then allowed to solidify into a gel. The solidified gel containing both the food and dsRNA can be cut into small pieces (approximately 1-10 mm, e.g. 1 mm, thick) using a razor blade, and can be used to feed mosquito larvae in water.

According to some embodiments, the nucleic acid agent is provided in amounts effective to reduce or suppress expression of at least one mosquito pathogen resistance gene product. As used herein “a suppressive amount” or “an effective amount” refers to an amount of dsRNA which is sufficient to downregulate (reduce expression of) the target gene by at least 20%, 30%, 40%, 50%, or more, say 60%, 70%, 80%, 90% or more even 100%.

Testing the efficacy of gene silencing can be effected using any method known in the art. For example, using quantitative RT-PCR measuring gene knockdown. Thus, for example, ten to twenty larvae from each treatment group can be collected and pooled together. RNA can be extracted therefrom and cDNA syntheses can be performed. The cDNA can then be used to assess the extent of RNAi by measuring levels of gene expression using qRT-PCR.

Reagents of the present invention can be packed in a kit including the nucleic acid agent (e.g. dsRNA), instructions for administration of the nucleic acid agent, construct or composition to mosquito larva.

Compositions of some embodiments of the invention may, if desired, be presented in a pack or dispenser device, which may contain one or more dosage forms containing the active ingredient. The pack may, for example, comprise metal or plastic foil, such as a blister pack. The pack or dispenser device may be accompanied by instructions for administration to the mosquito larva.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. 1., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Experimental Procedures

Gene Target Selection

Target genes are selected according to reported microarray and RNAseq experiments that compare populations of infected versus uninfected mosquitoes. A list of about 100 potential genes for target is generated. Genes from different functional categories are targeted, such as: metabolism (MET), immunity (IMM), cytoskeleton, cell membrane, cell motility and extracellular structures (C-CWCM-ES), post-translational modification, protein turnover, chaperone (PM-PT-C), signal transduction (ST), proteolysis (PROT), oxidoreductase activity (REDOX), transcription and translation (TT), diverse (DIV), transport (TR), cell-cycle (CC), energy production and conversion (EPC), chromatin structure and dynamics (CSD). The specific sequence for targeting is selected according to siRNA analysis available on-line, such as://www(dot)med(dot)nagoya-u(dot)ac(dot)jp/neurogenetics/i_Score/i_score(dot)html. The selected sequences are ordered synthetically and serve as template for in vitro reverse transcription reaction.

For example, RNAi pathway genes, including the sequence of the gene

AAEL011753 (r2d2) by 76-575 (SEQ ID NO: 304, one of the proteins of the silencing complex) is selected for targeting and dsRNA targeting same is generated as described below.

dsRNA Preparation

dsRNA preparation is performed by reverse transcription reaction using T7 primers, such as with the Ambion® MEGAscript® RNAi Kit. dsRNA integrity is verified on gel and purified by a column based method. The concentration of the dsRNA is evaluated both by Nano-drop and gel-based estimation. This dsRNA serves for the following experiments.

Bioassays

A. aegypti is reared at 27° C., 50% humidity, on a 16:8 L:D photoperiod. Females are fed warmed cattle blood through a stretched film. Mosquito eggs are allowed to develop for a minimum of one week, then are submerged in dechlorinated tap water to induce hatching. Larvae are maintained on a ground powder diet compromising dry cat food, dry rabbit chow, fish flakes and yeast.

Groups of 20 first instar larvae are soaked for 2 hr in 75 μl water containing 0.5 μg/μl dsRNA and 0.5% bromophenol blue. The larvae are photographed and the intensity of the dye in the gut is calculated using ImageJ image processing software (://rsbweb(dot)nih(dot)gov/ij/). The extent of dye in the gut is correlated with the extent of knockdown of the gene expression using quantitative reverse transcriptase PCR (see section below). Once it is determined that dsRNA is being ingested by larvae, subsequent dsRNA treatments are performed without the addition of the dye.

First instar larvae (less than 24 hr old) are treated in groups of 50 in a final volume 75 μl of dsRNA at a concentration of 0.5 μg/μl dsRNAs) in a 2 mL microfuge tube. Negative control larvae are treated with either water alone or with scrambled dsRNA, which has no homology with any mosquito genes and has no adverse effects on several other insects.

Larvae are soaked in the dsRNA solutions for 2 hr at 27° C., and then transferred to 12-well tissue culture plates, which are also maintained at 27° C., and are provided with a restricted diet on a daily basis. This amount of food is equivalent to half-rations of food per day typically enabled for most of the insects' population to develop to the pupal stage in 5 days. The reduced food during these bioassays slows their development and facilitates easier monitoring of differential growth rates and/or survivorship. Growth and/or survival of the larvae are observed over a 2-week period, by which time all non-treated larvae are pupated and have developed into adults. Once becoming adults, the mosquitoes are infected with viruses, and the extant of infection is tested.

Quantitative RT-PCR to Measure Gene Knockdown

Ten to 20 larvae from each treatment is collected and pooled together 3 days after the single 2 hr dsRNA soakings. RNA extractions and cDNA syntheses are performed. Only live insects are used for the RNA extractions, as the RNA in dead insects could have degraded. The cDNA from each replicate treatment is then used to assess the extent of RNAi by measuring levels of gene expression using qRT-PCR. Reactions are performed in triplicate and compared to an internal reference to compare levels of RNAi. Larva with decreased levels of a tested gene are allowed to pupate and become adult. The adult mosquitoes are further submitted to virus infection.

Virus and Mosquito Oral Infection

Viruses are cultured in Ae. albopictus C6/36 cells and high passage (25 passages) viruses are used in oral challenges as previously described [Salazar et al. (2007) BMC Microbiol 30: 7-9]. Specifically, about 350 adult females are fed either a virus-infected meal diluted 1:1 in cattle's blood or uninfected C6/36 cell culture medium diluted 1:1 in cattle's blood, respectively. Blood meals are measured for their viral titer. After blood feeding, 20 virus infected mosquitoes are sacrificed and viral titers are determined for each individual using a standard method as previously described [Hess et al. (2011) BMC Microbiol 11: 45]. Specifically, mosquito bodies are homogenized in 270 ml of Dulbecco's Modified Eagle Medium (DMEM) and then centrifuged to eliminate large debris particles. The supernatant are then further filtered and used in serial dilutions to infect monolayers of Vero cells. The lowest concentration infecting Vero cells is used to calculate the viral titer of virus infected mosquitoes.

### Results

### Use of Externally Ingested dsRNA to Increase Susceptibility of Mosquitoes to Human Pathogenic Viruses

A recently published RNAseq analysis describing mosquito transcriptional profiles during Dengue fever virus infection (DENVI) showed that all transcripts representing immunity-related genes with differential accumulation in midgut samples were always more abundant in control than DENV mosquitoes, supporting the conclusion that there is a suppression of the insect immune system following infection. This result may reflect the general ‘DENV downregulation trend” observed. A similar pattern was seen in carcass samples at early time points postinfection, but the opposite was observed at 14 days post infection (dpi), reflecting a possible change in immune modulation during the course of the infection [Bonizzoni et al. (2012) PLoS ONE 7(11): e50512].

The present inventors contemplate that feeding dsRNA to mosquitoes that will make them more susceptible to a pathogenic human virus that they carry means that only those mosquitoes that contract the virus will die from the dsRNA delivered.

Accordingly, genes to be targeted are selected, for example, as those whose products were more abundant in DENV as compared to control non-infected mosquitoes in carcass samples. Therefore, mosquitoes are fed with dsRNA targeting chromatin structure and dynamics (AAEL003673 [histone H4]; AAEL003689 [histone H4]; AAEL003669 [histone H2]), proteolysis (AAEL002610 [serine protease]), transcription and translation (AAEL005004) and immunity (AAEL011455 [CTLMA12]; AAEL007599, AAEL007585, AAEL012216, AAEL015312 [cathepsin B]; AAEL017536 [holotricin]).

Furthermore, genes to be targeted are selected, for example, as those whose transcript accumulation levels are higher in midgut samples of DENV as compared to control non-infected mosquitoes. Accordingly, mosquitoes are fed with dsRNA targeting genes linked to transcription and translation (AAEL003603), redox activity (AAEL007669) and to unknown functions (AAEL001702; AAEL017571).

Furthermore, genes to be targeted are selected, for example, as those who are more abundant in salivary glands of DENV as compared to control non-infected mosquitoes. Accordingly, mosquitoes are fed with dsRNA targeting immunity-related genes (AAEL015312 and AAEL012216, both encoding for cathepsin B). Furthermore, mosquitoes are fed with dsRNA targeting a total of 12 genes which had read coverage in salivary glands of DENV mosquitoes, but not in the salivary gland of control mosquitoes and are associated with various functions in metabolism (AAEL008418 [pyrroline-5-carboxylate reductase]), proteolysis (AAEL013857), the cytoskeleton (AAEL000335 [lamin]), redox activity (AAEL003211), chromatin structure and dynamics (AAEL003950 [helicase]), transcription and translation (AAEL002422 [cytoplasmic polyadenylation element binding protein]) and signal transduction (AAEL015328).

Moreover, when exposed to arboviruses mosquitoes respond with anti-microbial immune pathways like Janus kinase-signal transducer and activator of transcription (JAK/STAT) and Toll pathways, immune deficiency (IMD) and RNA interference (RNAi) machinery. Accordingly, mosquitoes are fed with dsRNA targeting these pathways. This process enables high viral titers and mosquito death.

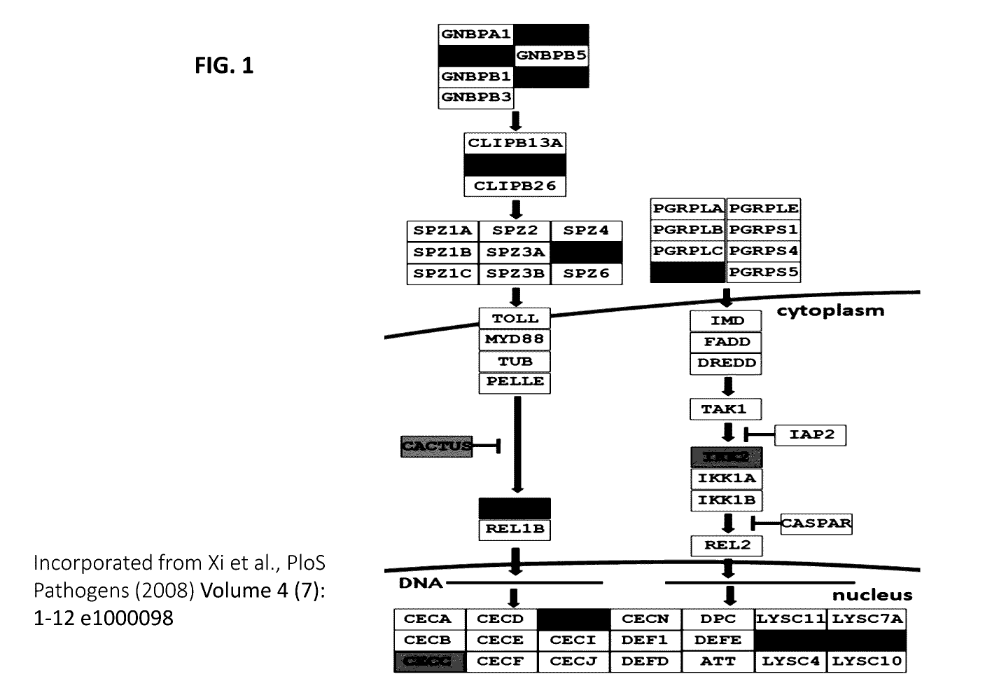

Thus, mosquitoes are fed with dsRNA targeting Toll pathway genes (see FIG. 1) as listed in Table 2, below.

TABLE 2
Toll pathway genes

| | gene | |
|---|---|---|
| Gene ID | name | functional group |
| AAEL000652 | GNBPA2 | pattern recognition receptor |
| AAEL009178 | GNBPB4 | pattern recognition receptor |
| AAEL007064 | GNBPB6 | pattern recognition receptor |
| AAEL003253 | CLIPB13B | signal modulation |
| AAEL001929 | SPZ5 | Toll |
| AAEL011608 | PGRPLD | pattern recognition receptor |
| AAEL007696 | REL1A | Toll |
| AAEL015515 | CECG | Effector |
| AAEL004522 | GAM | Effector |
| AAEL015404 | LYSC | Effector |

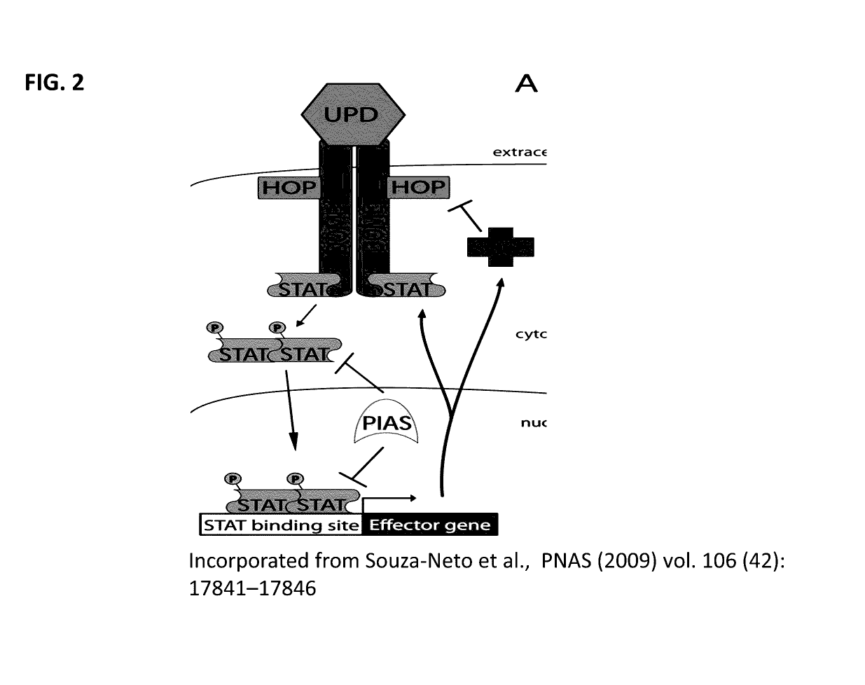

Mosquitoes are fed with dsRNA targeting JAK/STAT pathway genes (see FIG. 2) as listed in Table 3, below.

TABLE 3
JAK/STAT pathway genes

| Gene ID | gene name |
|---|---|
| AAEL012471 | DOME |
| AAEL012553 | HOP |
| AAEL009692 | STAT |
| AAEL006949, | SOCS16D |
| AAEL006936 | |
| AAEL000255 | SOCS44A |
| AAEL000393 | SOCS |
| AAEL015099 | SUMO |

Mosquitoes are fed with dsRNA targeting RNAi machinery including the gene AAEL011753 (r2d2), dcr2, and ago2.

The piRNA pathway, which has been suggested to function as an additional small RNA-mediated antiviral response to the known infection-induced siRNA response, is also targeted by the dsRNA. Exemplary genes which are targeted include those coding for the proteins Ago3, Ago4-like, Ago5-like, Armitage, Spn-E,Rm62-like. Accordingly mosquitoes are fed with dsRNA targeting these genes.

Other Pathways and Genes which are Targeted with the dsRNA

Mosquitoes are also fed with dsRNA targeting other pathways and genes, which may be involved in increasing susceptibility of the mosquitoes to viral infections. These include the genes listed in Table 4A, below.

TABLE 4A
Other pathways and genes which may be targeted

| Gene Family or Pathway | Dm | Ag | Aa | 1:1:1 | 1:1 | Total |
|---|---|---|---|---|---|---|
| Attacins (ATTs) | 4 | 1 | 1 | 0 | 0 | 6 |
| Caspases (CASPs) | 7 | 14 | 10 | 2 | 2 | 31 |
| Catalases (CATs) | 2 | 1 | 2 | 0 | 0 | 5 |
| Cecropins (CECs) | 5 | 4 | 10 | 0 | 1 | 19 |
| CLIP-domain Serine Proteases | 11 | 15 | 6 | 3 | 1 | 32 |
| A (CLIPAs) | | | | | | |
| CLIP-domain Serine Proteases | 14 | 20 | 36 | 3 | 5 | 70 |
| B (CLIPBs) | | | | | | |
| CLIP-domain Serine Proteases | 7 | 8 | 12 | 0 | 4 | 27 |
| C (CLIPCs) | | | | | | |
| CLIP-domain Serine Proteases | 10 | 7 | 8 | 5 | 0 | 25 |
| D (CLIPDs) | | | | | | |
| CLIP-domain Serine Proteases | 3 | 6 | 5 | 0 | 0 | 14 |
| E (CLIPEs) | | | | | | |
| C-Type Lectins (CTLs) | 34 | 25 | 39 | 9 | 1 | 98 |
| Defensins (DEFs) | 1 | 4 | 4 | 0 | 0 | 9 |
| Fibrinogen-Related proteins | 14 | 61 | 37 | 2 | 3 | 112 |
| (FREPs) | | | | | | |
| Galectins (GALEs) | 6 | 10 | 12 | 3 | 1 | 28 |
| Glutathione Peroxidases (GPXs) | 2 | 3 | 3 | 2 | 1 | 8 |
| Gram-Negative Binding | 3 | 7 | 7 | 1 | 4 | 17 |
| Proteins (GNBPs) | | | | | | |
| Heme Peroxidases (HPXs) | 10 | 18 | 12 | 8 | 1 | 40 |
| IMD Pathway Members | 5 | 5 | 6 | 4 | 0 | 16 |
| Inhibitors of Apoptosis | 4 | 8 | 5 | 4 | 0 | 17 |
| (IAPs) | | | | | | |
| JAK/STAT Pathway Members | 3 | 4 | 3 | 2 | 0 | 10 |
| Lysozymes (LYSs) | 13 | 8 | 7 | 1 | 1 | 28 |
| MD2-like Proteins (MLs) | 8 | 11 | 17 | 2 | 2 | 36 |
| Other Anti-microbial Peptides | 11 | 1 | 3 | 0 | 1 | 15 |
| (AMPs)* | | | | | | |
| Peptidoglycan Recognition | 13 | 7 | 8 | 5 | 0 | 28 |
| Proteins (PGRPs) | | | | | | |
| Prophenoloxidases (PPOs) | 3 | 9 | 10 | 0 | 3 | 22 |
| Rel-like NFkappa-B Proteins | 3 | 2 | 3 | 1 | 0 | 8 |
| (RELs) | | | | | | |
| Scavenger Receptors Class-A | 5 | 5 | 5 | 4 | 1 | 15 |
| (SCRAs) | | | | | | |
| Scavenger Receptors Class-B | 13 | 13 | 13 | 8 | 5 | 39 |
| (SCRBs) | | | | | | |
| Scavenger Receptors Class-C | 4 | 1 | 2 | 0 | 1 | 7 |
| (SCRCs) | | | | | | |
| Serine Protease Inhibitors | 30 | 17 | 23 | 2 | 14 | 70 |
| (SRPNs) | | | | | | |
| Späetzle-like Proteins (SPZs) | 6 | 6 | 9 | 4 | 0 | 21 |
| Superoxide Dismutatses (SODs) | 4 | 5 | 6 | 4 | 1 | 15 |
| Thio-Ester Containing | 6 | 13 | 8 | 1 | 1 | 27 |
| Proteins (TEPs) | | | | | | |
| Thioredoxin Peroxidases | 8 | 5 | 5 | 4 | 1 | 18 |
| (TPXs) | | | | | | |
| Toll Pathway Members | 4 | 4 | 4 | 4 | 0 | 12 |
| Toll Receptors (TOLLs) | 9 | 10 | 12 | 3 | 2 | 30 |
| Totals | 285 | 338 | 353 | 91 | 57 | 976 |

*Diptericins, Drosomycins, Drosocin, Metchnikowin, Gambicin, Holotricin

Taken together, these genes can serve as valid target for dsRNA silencing, thus reducing the mosquito's self-defense against the virus infection, causing the mosquito to be more susceptible to virus infection.

### Example 2

### Materials and Experimental Procedures

Mosquito Maintenance

Mosquitoes were taken from an Ae. aegypti colony of the Rockefeller strain, which were reared continuously in the laboratory at 28° C. and 70-80% relative humidity. Adult mosquitoes were maintained in a 10% sucrose solution, and the adult females were fed with sheep blood for egg laying. The larvae were reared on dog/cat food unless stated otherwise.

Introducing dsRNA into a Mosquito Larvae

Soaking with “Naked” dsRNA Plus Additional Larvae Feeding with Food-Containing dsRNA

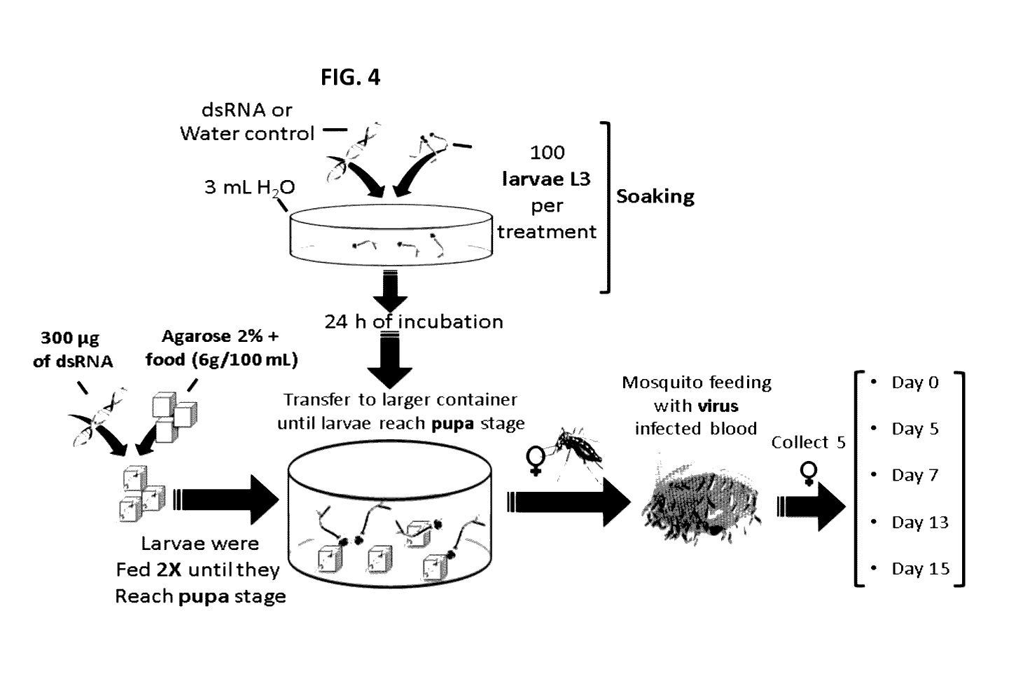

Third instar larvae were treated (in groups of 100 larvae) in a final volume of 3 mL of dsRNA solution in autoclaved water. Targets and dsRNA concentrations are shown in table 4B below. The control group was kept in 3 ml sterile water only. After soaking in the dsRNA solutions for 24 hr at 27° C., the larvae were transferred into new containers (300 larvae/1500 mL of chlorine-free tap water), and provided both agarose cubes containing 300 μg of dsRNA once a day (for a total of two days) and 6 mg/100 mL lab dog/cat diet (Purina Mills) suspended in water. As pupae developed, they were transferred to individual vials to await eclosion and sex sorting. For bioassays purpose only females up to five days old were used. See Flowchart in FIG. 4.

TABLE 4B
Targets and dsRNA concentrations

| dsRNA | Concentration (μg/μL per 100 larvae) |
|---|---|
| MyD88 (AAEL007768) | 0.2 |
| Rel-1A (AAEL007696) | 0.1 |
| AAEL003832 | 0.1 |
| AAEL000598 | 0.2 |
| AAEL007562 | 0.2 |
| AAEL010179 | 0.2 |

Preparation of Mosquito Larval Food Containing dsRNA

Cubes of dsRNA-containing mosquito food were prepared as follows: First, 300 μg of dsRNA were mixed with 30 μg of Polyethylenimine 25 kDa linear (Polysciences) in 200 μL of sterile water. Alternatively, 2 different dsRNA (150 μg of each) plus 30 μg of Polyethylenimine were mixed in 200 μL of sterile water. Then, a suspension of ground mosquito larval food (6 grams/100 mL) was prepared with 2% agarose (Fisher Scientific). The food/agarose mixture was heated to 55° C. and 200 μL of the mixture was then transferred to the tubes containing 200 μL of dsRNA+PEI or water only (control). The mixture was then allowed to solidify into a gel. The solidified gel containing both the food and dsRNA was cut into small pieces (approximately 1 mm thick) using a razor blade, which were then used to feed mosquito larvae in water.

RNA Isolation and dsRNA Production

Total RNA was extracted from groups of five Ae. aegypti fourth instar larvae and early adult male/female Ae. aegypti, using TRIzol (Invitrogen, Carlsbad, Calif., USA) according to the manufacturer's instructions. RNA was treated with amplification grade DNase I (Invitrogen) and 1 μg was used to synthesize cDNA using a First Strand cDNA Synthesis kit (Invitrogen). The cDNA served as template DNA for PCR amplification of gene fragments using the primers listed in Table 5, below. PCR products were purified using a QIAquick PCR purification kit (Qiagen). The MEGAscript RNAi kit (Ambion) was then used for in vitro transcription and purification of dsRNAs (Table 6, below).

TABLE 5
qPCR primers

| Target gene | Accession number | qPCR primers (5′-3′) |
|---|---|---|
| FHV RNA-1 | EF690537.1 | F: CCAGATCACCCGAACTGAAT |
| | | (SEQ ID NO: 1295) |
| | | R: AGGCTGTCAAGCGGATAGAA |
| | | (SEQ ID NO: 1296) |
| Argonaute-3 | XM_001652895.1 | F: TCGGCATTCGTAGCTTCGTT |
| AAEL007823 | | (SEQ ID NO: 1297) |
| | | R: GCAGCTGACAGTTTGCCTTC |
| | | (SEQ ID NO: 1298) |
| AuB | | F: CAGAATCCCAGACCCGGAAC |
| AAEL007698 | | (SEQ ID NO: 1299) |
| | | R: TTGGCGAAACCGTACCTTGA |
| | | (SEQ ID NO: 1300) |
| Cactus | XM_001650217.2 | F: ACTTTCCCTGGCCTTTCCAC |
| AAEL000709 | | (SEQ ID NO: 1301) |
| | | R: GCGAAACGTGAAGGTGCTAC |
| | | (SEQ ID NO: 1302) |
| MyD88 | XM_001658585.2 | F: TGCCGAGAACAGTGATCAGG |
| AAEL007768 | | (SEQ ID NO: 1303) |
| | | R: CTCAGATTTTTCGCCGGTGC |
| | | (SEQ ID NO: 1304) |
| AAEL007696 | XM_001652790.2 | F: GGACTCGTCGGAGCTGAAAT |
| Rel-1A | | (SEQ ID NO: 1305) |
| | | R: AACTGTCCGAGAGGGTTTCG |
| | | (SEQ ID NO: 1306) |
| AAEL003832 | XM_001657238.2 | F: TGAGTTTCTCGAGAGGAAAACCT |
| | | (SEQ ID NO: 1307) |
| | | R: TCACTACCCCTCCCTCGTTT |
| | | (SEQ ID NO: 1308) |
| AAEL000598 | XM_001649131.2 | F: TTCGCAGCTTTCGTCATGTG |
| | | (SEQ ID NO: 1309) |
| | | R: TTTCGAAACGGCGCAATCAC |
| | | (SEQ ID NO: 1310) |
| AAEL007562 | XM_001658400.1 | F: AGCTGCCATGTCTCAATCGT |
| | | (SEQ ID NO: 1311) |
| | | R: CCAGTTGGAAATTTCGCGGG |
| | | (SEQ ID NO: 1312) |
| AAEL010179 | XM_001654244.1 | F: TTCTGTTGGACGGCCCTTAC |
| | | (SEQ ID NO: 1313) |
| | | R: AGCCCGCAAACGGTGTAATA |
| | | (SEQ ID NO: 1314) |

TABLE 6
dsRNA sequences

| Target gene | Accession number | dsRNA sequence |
|---|---|---|
| Argonaute-3 | XM_001652895.1 | SEQ ID NO: 1315 |
| AAEL007823 | | |
| AuB | | SEQ ID NO: 1316 |
| AAEL007698 | | |
| Cactus | XM_001650217.2 | SEQ ID NO: 1317 |
| AAEL000709 | | |
| MyD88 | XM_001658585.2 | SEQ ID NO: 1318 |
| AAEL007768 | | |
| AAEL007696 | XM_001652790.2 | SEQ ID NO: 1319 |
| Rel1A | | |
| AAEL003832 | XM_001657238.2 | SEQ ID NO: 1320 |
| AAEL007562 | XM_001658400.1 | SEQ ID NO: 1321 |
| AAEL010179 | XM_001654244.1 | SEQ ID NO: 1322 |
| B2 FVH | X77156.1 | SEQ ID NO: 1323 |
| Dicer-2 | AY713296.1 | SEQ ID NO: 1324 |

qPCR Analysis

Approximately 1000 ng first-strand cDNA obtained as described previously was used as template. The qPCR reactions were performed using SYBR® Green PCR Master Mix (Applied Biosystems) following the manufacturer's instructions. Briefly, approximately 50 ng/μl cDNA and gene-specific primers (600 nM) were used for each reaction mixture. qPCR conditions used were 10 min at 95° C. followed by 35 cycles of 15 s at 94° C., 15 s at 54° C. and 60 s at 72° C. The ribosomal protein S7 and tubulin were used as the reference gene to normalize expression levels amongst the samples. Raw quantification cycle (Cq) values normalized against those of the tubulin and S7 standards were then used to calculate the relative expression levels in samples using the 2^{-ΔΔCt} method [Livak & Schmittgen, (2001) Methods. 25(4):402-8]. Results (mean±SD) are representative of at least two independent experiments performed in triplicate.

Cells and Preparation of Flock House Virus (FHV) Stocks

D. melanogaster cells (S2) were grown at 26° C. in Schneider's insect cell medium (Gibco, Life Technologies) supplemented with 10% fetal bovine serum (FBS). FHV stocks were prepared by propagation in S2 cells at a multiplicity of infection (MOI) of 5 for 72 hours. Then, cell-free supernatants were collected, aliquoted and stored at −80° C. until the moment of use. Viral loads were quantified in the S2-culture supernatants using a quantitative Real-Time PCR. Briefly, total viral RNA purified from 1×10⁸ PFU of FHV were 10-fold serially diluted to generate a standard curve. The viral RNA was purified using the QIAamp Viral RNA minikit (QIAGEN; Hamburg, Germany). Viral RNA was converted in cDNA using Improm II kit (Promega) and the quantitative PCR reaction was carried out with the Power SYBR Green Master mix (Invitrogen, Life Technologies) in a 7500-Real time PCR System (Applied Biosystems, Life Technologies). The primer sequences used for FHV detection were detailed in Table 5, above.

Infection of Mosquitoes with FHV

Female Aedes aegypti mosquitoes (Rockefeller strain) were infected with FHV by two different methods. In the first one, mosquitoes were fed an artificial blood meal mixed with FHV-infected S2 supernatants at a 1:1 ratio (virus titres were 1-2×10⁸ PFU/mL) through a pork gut membrane on a water-jacketed membrane feeder as previously described [Rutledge et al. (1964) Mosq News. 24:407-419], for 20 minutes, and then kept in breeding cages up to 15 days postinfection. Control mosquitoes were fed uninfected blood. In the second method of infection, the same source of FHV was diluted at 1:1 ratio in a 10%-solution of sugar. The mixture was then adsorbed in filter papers and placed into the breeding cages. The exposure to mosquitoes lasted 20 minutes. Control mosquitoes were exposed to sugar adsorbed in the filter papers.

Determination of Viral Loads in Infected Mosquitoes

Mosquitoes infected with FHV were collected at different time points postinfection, as indicated. Total RNA was extracted with TRIzol (Invitrogen) according to the manufacturer's protocol. cDNAs were synthesized by using Improm II Reverse transcriptase (Promega) and oligo dT (Thermo Scientific). Real-time quantitative PCRs were carried out using Power SYBR green Master Mix (Life technologies) and specific primers to FHV RNA1 (Table 6, above). The relative viral loads were estimated by the 2^{-ΔΔCT} method, and normalized to a mosquito endogenous control (tubulin).

### Results

### Use of dsRNA to Increase Susceptibility of Ae. Aegypti Mosquitoes to Flock House Virus (FHV)

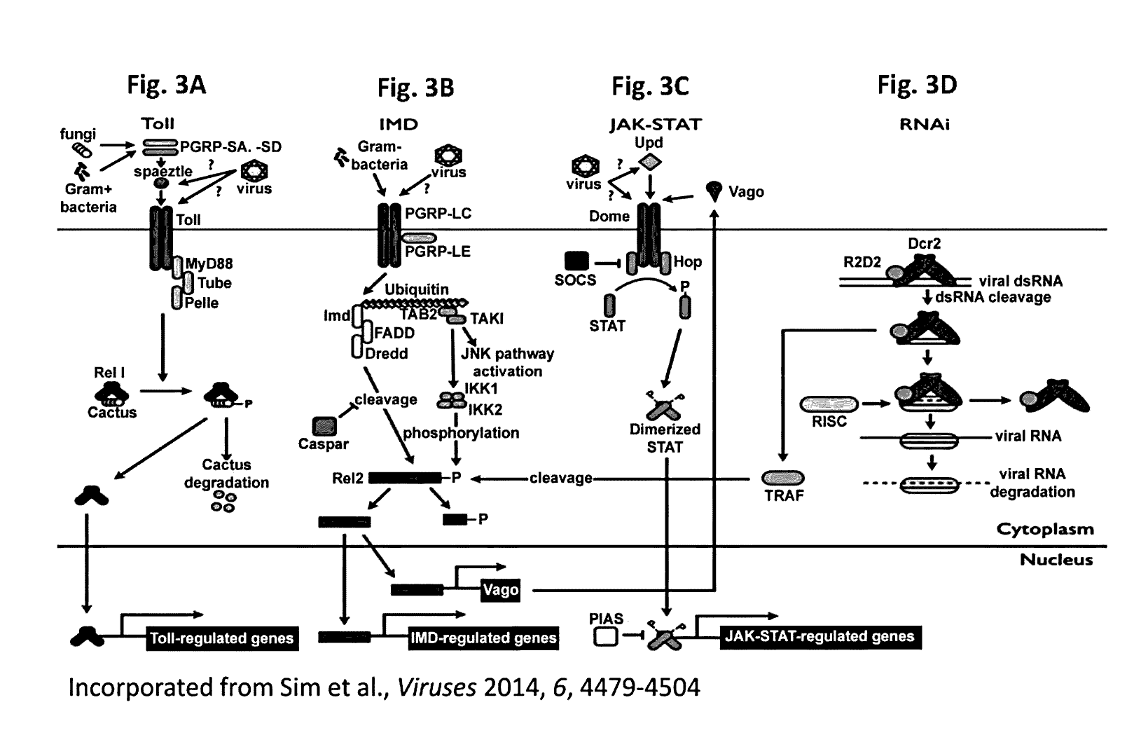

In this study, the present inventors explored the infection of Ae. aegypti mosquitoes with Flock House virus (FHV) as an experimental model to increase the mosquito susceptibility to virus infection. The purpose of this experiment was to treat mosquito larvae using dsRNA in order to increase virus replication inside mosquitoes. To do so, the present inventors designed dsRNA sequences to target specifically MYD88, Rel1A and defensin anti-microbial peptide. FIGS. 3A-D illustrate mosquito-signaling pathways that have been implicated in the antiviral defense, namely the Toll, immune deficiency (IMD), Janus kinase/signal transducers and activators of transcription (JAK-STAT) and RNA interference (RNAi) pathways.

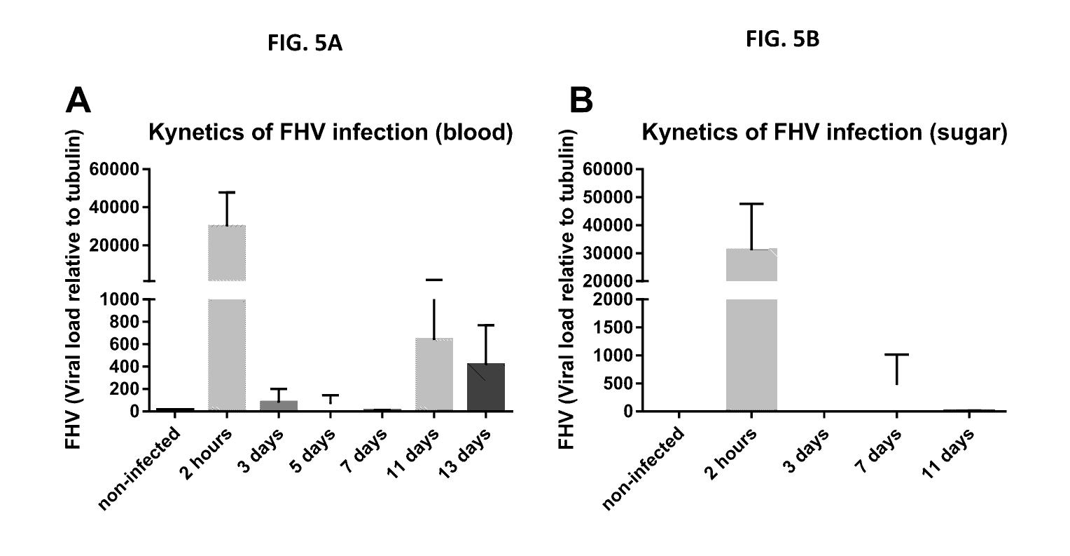

It has been shown previously that FHV replicates in four species of mosquito, including Ae. aegypti. In this study, FHV growth was first monitored in Ae. aegypti mosquitoes at different intervals (2 hours, 3, 5, 7, 11 and 13 days) following an infectious blood meal or infectious sugar meal. The virus titer was high in both methods of infection 2 hours after infection and decreased thereafter until day 7 (FIGS. 5A-B). However, only in the group infected with blood meal, the virus titers rise again 11 and 13 days postinfection (FIG. 5A).

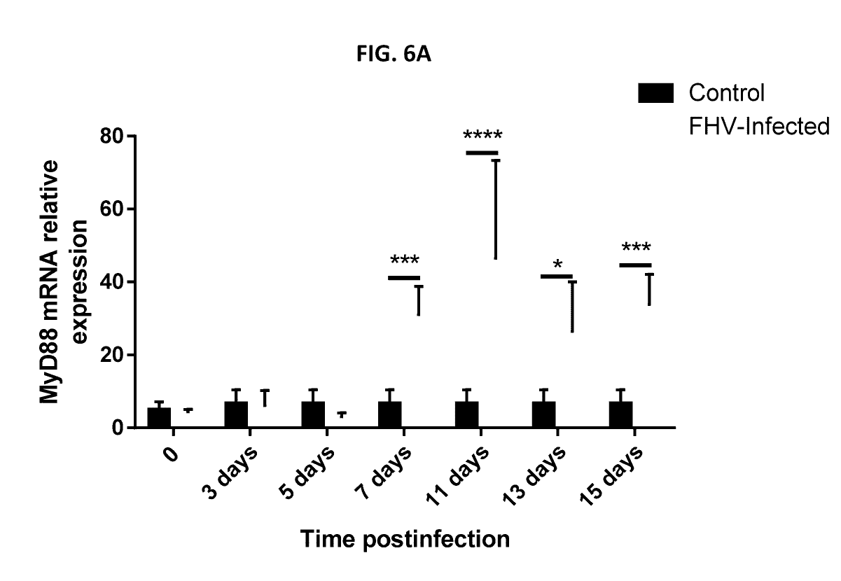

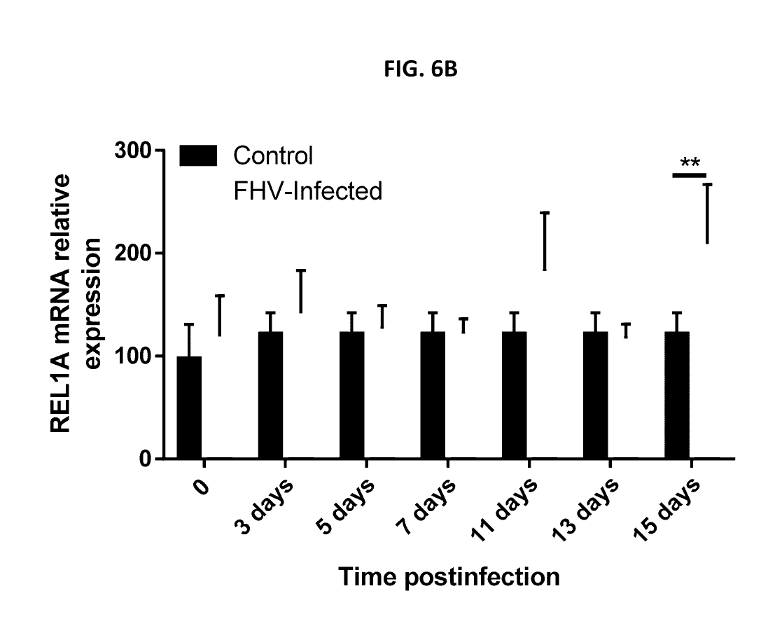

In order to evaluate the activation of immune response mechanism after FHV infection, the expression level of MYD88 and Rel1A were also evaluated in mosquitoes at different intervals (2 hours, 3, 5, 7, 11, 13 and 15 days) following an infectious blood meal. Interestingly, the mRNA levels of MYD88 (FIG. 6A) and Rel1A (FIG. 6B) increased at 7 and 15 days post infection, respectively.

It has been previously shown that MYD88-silenced mosquitoes (after intrathoracic inoculation of dsRNA into the mosquito) prior to dengue virus infection resulted in an increase of the virus load by 2.7 times compared to the GFP dsRNA control. On the other hand, cactus gene silencing reduced the extent of dengue infection in the midgut by 4.0-fold when compared to the GFP dsRNA control [PLoS Pathog. 2008 Jul. 4; 4(7):e1000098]. In the current experiment, third instar larvae were treated with dsRNA against MYD88 and Cactus. Larvae were reared until adult mosquitoes and then received an infectious blood meal. Using this approach, an increase in virus load was found two hours after infection in the MYD88, Cactus and AAEL007562 dsRNA-treated group (FIGS. 7A, 7B and 7E).

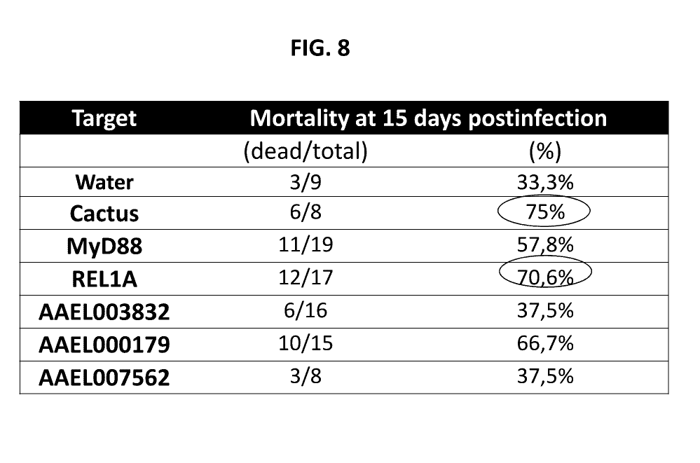

At later time point (15 days) Rel1A and cactus-treated mosquitoes displayed the highest mortality rates (FIG. 8).

Furthermore, MYD88 dsRNA-treated mosquitoes displayed a higher infection rate at 7 and 15 days postinfection (FIG. 9A). When the viral load was analyzed at 15 days postinfection, dead mosquitoes from MYD88 dsRNA-treated group displayed higher virus titer as compared with live mosquitoes (FIG. 9B). In addition, a decreased MyD88 expression level was detected in dead mosquitoes from the MYD88 dsRNA-treated group as compared to live mosquitoes (FIG. 9C). Similar results were obtained with Rel1A dsRNA-treated mosquitoes at 15 days postinfection (FIGS. 10A-C).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of controlling a pathogenically infected mosquito, the method comprising administering to a larva of a mosquito an isolated nucleic acid agent comprising a nucleic acid sequence which specifically downregulates an expression of at least one mosquito pathogen resistance gene product of said mosquito, wherein downregulation of said expression of said at least one mosquito pathogen resistance gene in said larvae renders an adult stage of said mosquito lethally susceptible to said pathogen, thereby controlling said pathogenically infected mosquito.

2. The method of claim 1, wherein said mosquito comprises a female mosquito being capable of transmitting a disease to a mammalian organism.

3. The method of claim 1, wherein said mosquito is of a species selected from the group consisting of Aedes aegypti, Aedes albopictus and Anopheles gambiae.

4. The method of claim 1, wherein said administering comprises feeding, spraying or soaking.

5. The method of claim 1, wherein said administering comprises soaking said larva with said isolated nucleic acid agent for about 12-48 hours.

6. The method of claim 5, wherein said larva comprises third instar larva.

7. The method of claim 5, further comprising feeding said larva with said isolated nucleic acid agent until said larva reaches pupa stage.

8. The method of claim 1, wherein said pathogenically infected mosquito carries an infection selected from the group consisting of a viral infection, a nematode infection, a protozoa infection and a bacterial infection.

9. The method of claim 8, wherein said viral infection is caused by an arbovirus.

10. The method of claim 9, wherein said arbovirus is selected from the group consisting of an alphavirus, a flavivirus, a bunyavirus and an orbivirus.

11. The method of claim 9, wherein said arbovirus is selected from the group consisting of a La Crosse encephalitis virus, an Eastern equine encephalitis virus, a Japanese encephalitis virus, a Western equine encephalitis virus, a St. Louis encephalitis virus, a Tick-borne encephalitis virus, a Ross River virus, a Venezuelan equine encephalitis virus, a Chikungunya virus, a West Nile virus, a Dengue virus, a Yellow fever virus, a Bluetongue disease virus, a Sindbis Virus and a Rift Valley Fever virus a Colorado tick fever virus, a Murray Valley encephalitis virus, an Oropouche virus and a Flock House virus.

12. The method of claim 8, wherein said protozoa infection is caused by a Plasmodium.

13. The method of claim 8, wherein said protozoa infection causes malaria.

14. The method of claim 8, wherein said nematode infection is caused by a Heartworm (Dirofilaria immitis) or a Wuchereria bancrofti.

15. The method of claim 8, wherein said nematode infection causes Heartworm Disease.

16. A mosquito larva-ingestible compound comprising an isolated nucleic acid agent comprising a nucleic acid sequence which specifically downregulates an expression of at least one mosquito pathogen resistance gene product in a mosquito and a microorganism or algae on which mosquito larva feed.

17. The mosquito larva-ingestible compound of claim 16 formulated as a solution.

18. The mosquito larva-ingestible compound of claim 16 formulated in a solid or semi-solid formulation.

19. The mosquito larva-ingestible compound of claim 18, wherein said semi-solid formulation comprises an agarose.

20. The mosquito larva-ingestible compound of claim 16, wherein said microorganism is selected from the group consisting of a bacteria and a water surface microorganism.

21. The method of claim 1, wherein said mosquito pathogen resistance gene is selected from the group consisting of a RNA interference related gene, a piRNA pathway related gene, an immunity related gene, a metabolism related gene, a cytoskeleton related gene, a cell membrane related gene, a cell motility related gene, an extracellular structure related gene, a post-translational modification related gene, a protein turnover related gene, a chaperone related gene, a signal transduction related gene, a proteolysis related gene, an oxidoreductase activity related gene, a transcription related gene, a translation related gene, a diverse related gene, a transport related gene, a cell-cycle related gene, an energy production and conversion related gene, a chromatin structure and dynamics related gene, a Toll related gene and a JAK/STAT related gene.

22. The method of claim 1, wherein said mosquito pathogen resistance gene is selected from the group consisting of AAEL003673 [histone H4], AAEL003689 [histone H4], AAEL003669 [histone H2], AAEL002610 [serine protease], AAEL005004, AAEL011455 [CTLMA12], AAEL007599, AAEL007585 [cathepsin B], AAEL017536 [holotricin], AAEL003603, AAEL007669, AAEL001702, AAEL017571, AAEL015312 [cathepsin B], AAEL012216 [cathepsin B], AAEL008418 [pyrroline-5-carboxylate reductase]), AAEL013857, AAEL000335 [lamin], AAEL003211, AAEL003950 [helicase], AAEL002422 [cytoplasmic polyadenylation element binding protein], AAEL015328, AAEL000652 [GNBPA2], AAEL009178 [GNBPB4], AAEL007064 [GNBPB6], AAEL003253 [CLIPB13B], AAEL001929 [SPZ5], AAEL011608 [PGRPLD], AAEL007696 [REL1A], AAEL015515 [CECG], AAEL004522 [GAM], AAEL015404 [LYSC], AAEL012471 [DOME], AAEL012553 [HOP], AAEL009692 [STAT], AAEL006949 [SOCS16D], AAEL006936 [SOCS16D], AAEL000255 [SOCS44A], AAEL000393 [SOCS], AAEL015099 [SUMO], AAEL011753 (r2d2), AAEL006794 (dcr2), AAEL017251 (ago2), AAEL007823 (Ago3), AAEL013235 (Spn-E), AAEL007698 (AuB), AAEL000709 (Cactus), AAEL007768 (MyD88), AAEL003832, AAEL007562, AAEL000598 and AAEL010179.

23. The method of claim 1, wherein said mosquito pathogen resistance gene is selected from the group consisting of AAEL007768 (MyD88), AAEL000709 (Cactus), AAEL007698 (AuB), AAEL003832, AAEL007562, Rel1A (AAEL007696), AAEL000598 and AAEL010179.

24. An isolated nucleic acid agent comprising a polynucleotide expressing a nucleic acid sequence which specifically downregulates an expression of at least one mosquito pathogen resistance gene selected from the group consisting of AAEL003673 [histone H4], AAEL003689 [histone H4], AAEL003669 [histone H2], AAEL002610 [serine protease], AAEL005004, AAEL011455 [CTLMA12], AAEL007599, AAEL007585 [cathepsin B], AAEL017536 [holotricin], AAEL003603, AAEL007669, AAEL001702, AAEL017571, AAEL015312 [cathepsin B], AAEL012216 [cathepsin B], AAEL008418 [pyrroline-5-carboxylate reductase]), AAEL013857, AAEL000335 [lamin], AAEL003211, AAEL003950 [helicase], AAEL002422 [cytoplasmic polyadenylation element binding protein], AAEL015328, AAEL000652 [GNBPA2], AAEL009178 [GNBPB4], AAEL007064 [GNBPB6], AAEL003253 [CLIPB13B], AAEL001929 [SPZ5], AAEL011608 [PGRPLD], AAEL007696 [REL1A], AAEL015515 [CECG], AAEL004522 [GAM], AAEL015404 [LYSC], AAEL012471 [DOME], AAEL012553 [HOP], AAEL009692 [STAT], AAEL006949 [SOCS16D], AAEL006936 [SOCS16D], AAEL000255 [SOCS44A], AAEL000393 [SOCS], AAEL015099 [SUMO], AAEL011753 (r2d2), AAEL006794 (dcr2), AAEL017251 (ago2), AAEL007823 (Ago3), AAEL013235 (Spn-E), AAEL007698 (AuB), AAEL000709 (Cactus), AAEL007768 (MyD88), AAEL003832, AAEL007562, AAEL010179 and AAEL000598.

25. An isolated nucleic acid agent comprising a polynucleotide expressing a nucleic acid sequence which specifically downregulates an expression of at least one mosquito pathogen resistance gene selected from the group consisting of AAEL007768 (MyD88), AAEL000709 (Cactus), AAEL007698 (AuB), AAEL003832, AAEL007562, Rel1A (AAEL007696), AAEL010179 and AAEL000598.

26. A nucleic acid construct comprising a nucleic acid sequence encoding the isolated nucleic acid agent of claim 24.

27. A cell comprising the isolated nucleic acid agent of claim 24.

28. The cell of claim 27 selected from the group consisting of a bacterial cell and a cell of a water surface microorganism.

29. A mosquito larva-ingestible compound comprising the cell of claim 27.

30. The cell of claim 27, wherein said nucleic acid agent is a dsRNA.

31. The cell of claim 30, wherein said dsRNA is a naked dsRNA.

32. The cell of claim 30, wherein said dsRNA comprises a carrier.

33. The cell of claim 32, wherein said carrier comprises a polyethyleneimine (PEI).

34. The cell of claim 30, wherein said dsRNA is effected at a dose of 0.001-1 μg/μL for soaking or at a dose of 1 pg to 10 μg/larvae for feeding.

35. The cell of claim 30, wherein said dsRNA is selected from the group consisting of SEQ ID NOs: 1315-1324 and 1330.

36. The cell of claim 30, wherein said dsRNA is selected from the group consisting of siRNA, shRNA and miRNA.

37-40. (canceled)

41. The method of claim 1, wherein said isolated nucleic acid agent is comprised in a cell.
